(19) United States
(12) Patent Application Publication
(10) Pub. No.: US 2024/0271221 A1
(43) Pub. Date: Aug. 15, 2024

(54) METHODS FOR ANALYSIS OF CELL-FREE NUCLEIC ACIDS IN URINE

(71) Applicant: GRAIL, LLC, Menlo Park, CA (US)

(72) Inventors: Matthew Larson, Amherst, MA (US); Archana Shenoy, Mountain View, CA (US); Sarah Stuart, Pacifica, CA (US); Kelly McClintock, Menlo Park, CA (US); Siddhartha Bagaria, San Francisco, CA (US)

(21) Appl. No.: 18/417,800

(22) Filed: Jan. 19, 2024

Related U.S. Application Data
(60) Provisional application No. 63/480,934, filed on Jan. 20, 2023.

(51) Int. Cl. C12Q 1/6886; A61K 45/06; C12N 15/10; C12Q 1/34; C12Q 1/6806; C12Q 1/6809; C12Q 1/6844; C12Q 1/6874; G16H 50/20

(52) U.S. Cl. CPC C12Q 1/6886; A61K 45/06; C12N 15/1006; C12N 15/1017; C12Q 1/34; C12Q 1/6806; C12Q 1/6809; C12Q 1/6844; C12Q 1/6874; G16H 50/20; C12Q 2600/112; C12Q 2600/154; G01N 2333/978

(57) ABSTRACT

In various aspects, the present disclosure provides methods, compositions, reactions mixtures, kits, and systems for analysis of cell-free nucleic acid molecules (e.g., cfRNA and/or cfDNA) from a urine sample. In some embodiments, the analysis is an analysis of methylation patterns in target genomic regions among cfDNA fragments in a urine sample. In some embodiments, compositions include a plurality of different bait oligonucleotides. Methods for the detection of cancer of various cancer types are also provided.

### CROSS-REFERENCE TO RELATED APPLICATION

This application claims priority to U.S. Provisional Application Ser. No. 63/480,934, filed on Jan. 20, 2023, the disclosure of which is hereby incorporated by reference in its entirety.

### BACKGROUND

Analysis of nucleic acids, such as circulating cell-free nucleic acids (e.g., cell-free DNA (cfDNA) and cell-free RNA (cfRNA)), using next generation sequencing (NGS) is recognized as a valuable method for characterizing various sample types. For example, such analyses are useful as a diagnostic tool for detection and diagnosis of cancer. These analytes can also be valuable in improving our fundamental understanding of basic biology.

DNA methylation plays an important role in regulating gene expression. Aberrant DNA methylation has been implicated in many disease processes, including cancer, DNA methylation profiling using methylation sequencing (e.g., whole genome bisulfite sequencing (WGBS)) is increasingly recognized as a valuable diagnostic tool for detection, diagnosis, and/or monitoring of cancer. For example, specific patterns of differentially methylated regions may be useful as molecular markers for various diseases.

However, WGBS is not ideally suitable for a product assay. The reason is that the vast majority of the genome is either not differentially methylated in cancer, or the local CpG density is too low to provide a robust signal. Only a few percent of the genome is likely to be useful in classification. These issues are compounded when working with sample types that are less rich in cell-free nucleic acids, such as urine.

### SUMMARY

For at least the above reasons, there remains a need for a cost-effective methods and compositions for the analysis of cell-free nucleic acid molecules in urine. Various aspects of the present disclosure address this need, and provide other advantages as well.

Early detection of cancer in subjects is important as it allows for earlier treatment and therefore a greater chance for survival. Targeted detection of cancer-specific methylation patterns using cell-free DNA (cfDNA) fragments can make early detection of cancer possible by providing cost-effective and non-invasive method for getting information relevant to detecting the presence or absence of cancer, a cancer tissue of origin, or cancer type, By using a targeted genomic region panel rather than sequencing all nucleic acids in a test sample, also known as “whole genome sequencing,” the method can increase sequencing depth of the target regions, Including methylation markers for several different types of cancer permits more efficient use of sample and reagents, as compared to conducting multiple separate assays for different types of cancer. However, it can be advantageous to limit the total genomic sequence coverage for capture, sequencing, and/or computational efficiency.

Towards that end, the present description provides cancer assay panels (alternatively referred to as “bait sets”) for detection of cancer-specific methylation patterns in targeted genomic regions, along with methods of using the cancer assay panels for detection of cancer, a cancer type, and/or cancer tissue of origin (TOO). The methods described herein further include methods of designing probes to enrich for cfDNA corresponding to or derived from selected genomic regions efficiently without pulling down an excessive amount of undesired DNA. Also provided are methods for the analysis of cfDNA and other cell-free nucleic acids in urine samples in particular.

In one aspect, provided herein are methods of sequencing cell-free nucleic acid molecules of a subject. In some embodiments, the method comprises: (a) treating a urine sample to inhibit cell lysis: (b) separating cell-free nucleic acid molecules in the treated urine sample from cells in the treated urine sample, thereby producing a purified urine sample comprising the cell-free nucleic acid molecules: (c) concentrating the cell-free nucleic acid molecules in the purified urine sample by passing at least a portion of the purified urine sample through a filter, wherein (i) the concentrating producing a filtrate and a retained urine sample, and (ii) the retained urine sample comprises an increased concentration of cell-free nucleic acid molecules: (d) isolating cell-free nucleic acid molecules from the retained urine sample; and (e) sequencing the isolated cell-free nucleic acid molecules.

In some embodiments, treating the urine sample to inhibit cell lysis comprises contacting the urine sample with a one or more preservative reagent. In some embodiments, the treating includes treatment with a nuclease inhibitor, a formaldehyde quencher, or both. In some embodiments, the treating comprises contacting the urine sample with a composition comprising: (i) imidazolidinyl urea. EDTA, glycine, or a combination thereof; or (ii) sodium azide. EDTA, or a combination thereof. In some embodiments, the separating comprises centrifugation to pellet cells in the treated urine sample. In some embodiments, the filter is substantially impermeable to passage of cell-free nucleic acids, and is substantially permeable to salts in the purified urine sample. In some embodiments, the filter comprises a rated molecular weight cutoff of 10 kD. 5 kD. 3 kD, or lower. In some embodiments, the retained urine sample has a concentration that is increased by at least 2-fold, at least 5-fold, at least 10-fold, or at least 15-fold compared to the purified urine sample. In some embodiments, the retained urine sample has a volume that is at least 50%, at least 75%, or at least 90% lower compared to the volume of the treated urine sample. In some embodiments, the volume of the treated urine sample is 20 mL, 30 mL, 40 mL, 50 mL, or more. In some embodiments, the method further comprises freezing the retained urine sample. In some embodiments, the treating is completed within 120, 60, or 30 minutes after collection of the urine sample; and optionally wherein the separating and the concentrating are completed within 7 days after collection.

In some embodiments, the method further comprises amplifying one or more of the isolated cell-free nucleic acid molecules. In some embodiments, the method further comprises capturing the isolated cell-free nucleic acid molecules, or amplification products thereof, by hybridization to bait oligonucleotides. In some embodiments, the method further comprises separating bait-bound cell-free nucleic acid molecules from unbound cell-free nucleic acid molecules.

In some embodiments, each bait oligonucleotide hybridizes to a target genomic region that is differentially methylated in a cancer sample relative to a non-cancer sample. In some embodiments, the differential methylation comprises at least 80% of CpG sites in the target genomic region being methylated or unmethylated. In some embodiments, the cancer is a bladder cancer, a prostate cancer, or a kidney cancer. In some embodiments, each bait oligonucleotide hybridizes to a target genomic region comprising at least five methylation sites. In some embodiments, each bait oligonucleotide hybridizes to a target genomic region comprising a target sequence of a gene selected from Table 1, and wherein the target sequence is at least 25, at least 35, or at least 45 nucleotides in length. In some embodiments, the target genomic region comprises a target sequence of a gene selected from TWIST1, EOMES, HOXA9, POU4F2, and ZNF154. In some embodiments, the bait oligonucleotides collectively hybridize to target sequences from at least 10 genes in Table 1. In some embodiments, the bait oligonucleotides collectively hybridize to target sequences from: (a) genes in Tables 2 or 3; (b) genes in Table 4; or (c) genes in Table 5.

In some embodiments, the cell-free nucleic acid molecules comprise cell-free DNA (cfDNA). In some embodiments, the method further comprises deaminating the cfDNA isolated in step (d) to produce converted cfDNA molecules: optionally wherein the deaminating comprises treatment with a cytosine deaminase or bisulfite.

In some embodiments, the method further comprises diagnosing a cancer in the subject. In some embodiments, the cancer is a bladder cancer, a prostate cancer, or a kidney cancer. In some embodiments, the method further comprises treating the cancer in the subject. In some embodiments, the treating comprises surgical resection, radiation therapy, chemotherapy, and/or immunotherapy.

In one aspect, provided herein are methods of detecting cancer cells in a subject. In some embodiments, the method comprises (a) capturing converted cell-free DNA (cfDNA) fragments from a urine sample of the subject, or amplification products thereof, wherein: (i) the bait oligonucleotide composition comprises a plurality of different bait oligonucleotides: (ii) each bait oligonucleotide of the plurality of different bait oligonucleotides hybridizes to a target sequence of a gene selected from Table 1, wherein the target sequence is at least 25 nucleotides in length: (b) separating bait-bound DNA from unbound DNA: (c) sequencing the separated DNA to produced sequencing reads; and (d) detecting the cancer cells with a trained classifier.

In some embodiments, the bait oligonucleotides are at least 45 nucleotides in length. In some embodiments, the trained classifier detects a number of sequencing reads above a threshold for one or more of the target sequences that are identified as hypermethylated and/or hypomethylated in the cfDNA fragments. In some embodiments, the trained classifier discriminates a subject with cancer from a subject without cancer with a defined specificity. In some embodiments, the classifier is a mixture model classifier. In some embodiments, the defined specificity is 0.900 or higher. In some embodiments, the application of the trained classifier further comprises a sensitivity of 30% or more. In some embodiments, the bait oligonucleotides collectively hybridize to target sequences from at least 10 genes in Table 1. In some embodiments, at least one of the bait oligonucleotides hybridizes to a target sequence of a gene selected from TWIST1, EOMES, HOXA9, POU4F2, and ZNF154. In some embodiments. (a) the cancer cells are bladder cancer cells, and the bait oligonucleotides collectively hybridize to target sequences from genes of Tables 2 or 3: (b) the cancer cells are prostate cancer cells, and the bait oligonucleotides collectively hybridize to target sequences from genes of Table 4; or (c) the cancer cells are kidney cancer cells, and the bait oligonucleotides collectively hybridize to target sequences from genes of Table 5.

In some embodiments, the converted cfDNA molecules comprise cfDNA treated with a cytosine deaminase or bisulfite. In some embodiments, each bait oligonucleotide is conjugated to a solid surface or to a non-nucleotide affinity moiety. In some embodiments, the differential methylation comprises hypermethylation in a cancer sample relative to a non-cancer sample. In some embodiments, each target genomic region comprises at least five methylation sites.

In some embodiments, the method further comprises obtaining the converted cfDNA fragments or amplification products thereof, wherein the obtaining further comprises: (i) treating a urine sample to inhibit cell lysis: (ii) separating cfDNA fragments in the treated urine sample from cells in the treated urine sample, thereby producing a purified urine sample comprising the cfDNA fragments: (iii) concentrating the cfDNA fragments in the purified urine sample by passing at least a portion of the purified urine sample through a filter, wherein the concentrating producing a filtrate and a retained urine sample, and wherein the retained urine sample comprises an increased concentration of cfDNA fragments; and (iv) isolating cfDNA fragments from the retained urine sample. In some embodiments, the method further comprises (v) amplifying one or more of the isolated cfDNA fragments.

In some embodiments, treating the urine sample to inhibit cell lysis comprises contacting the urine sample with a one or more preservative reagent. In some embodiments, the treating includes treatment with a nuclease inhibitor, a formaldehyde quencher, or both. In some embodiments, the treating comprises contacting the urine sample with a composition comprising: (a) imidazolidinyl urea. EDTA, glycine, or a combination thereof; or (b) sodium azide. EDTA, or a combination thereof. In some embodiments, the separating comprises centrifugation to pellet cells in the treated urine sample. In some embodiments, the filter is substantially impermeable to passage of cell-free nucleic acids, and is substantially permeable to salts in the purified urine sample. In some embodiments, the filter comprises a rated molecular weight cutoff of 10 kD. 5 kD. 3 kD, or lower. In some embodiments, the retained urine sample has a concentration that is increased by at least 2-fold, at least 5-fold, at least 10-fold, or at least 15-fold compared to the purified urine sample. In some embodiments, the retained urine sample has a volume that is at least 50%, at least 75%, or at least 90% lower compared to the volume of the treated urine sample. In some embodiments, the volume of the treated urine sample is 20 mL, 30 mL, 40 mL, 50 mL, or more. In some embodiments, the method further comprises freezing the retained urine sample. In some embodiments, the treating is completed within 120, 60, or 30 minutes after collection of the urine sample; and optionally wherein the separating and the concentrating are completed within 7 days after collection.

In some embodiments, the method further comprises diagnosing a cancer in the subject. In some embodiments, the cancer is a bladder cancer, a prostate cancer, or a kidney cancer. In some embodiments, the method further comprises treating the cancer in the subject. In some embodiments, the treating comprises surgical resection, radiation therapy, chemotherapy, and/or immunotherapy.

In one aspect, provided herein are methods of treating cancer in a subject. In some embodiments, the method comprises selecting a subject having or being at increased risk of developing cancer, and administering a treatment to the subject, wherein: (a) the selecting comprises identifying the subject as the source of a urine cell-free DNA (cfDNA) sample comprising one or more differentially methylated target genomic regions above a threshold level for the presence of the cancer: (b) the one or more target genomic regions comprise one or more target sequences of one or more genes selected from Table 1: (c) each target sequence is at least 25 nucleotides in length: (d) the cancer is bladder cancer, prostate cancer, or kidney cancer; and (e) the treatment comprises surgical resection, radiation therapy, chemotherapy, immunotherapy, or any combination thereof.

In some embodiments, the threshold level for the presence of the cancer is a level for reference samples from subjects having the cancer. In some embodiments, the threshold level for the presence of the cancer is determined by a classifier trained on sequencing reads for converted DNA from subjects having the cancer In some embodiments, the one or more target genomic regions comprise target sequences from at least 10 genes in Table 1. In some embodiments, wherein the one or more target genomic regions comprise a target sequence of a gene selected from TWIST1, EOMES, HOXA9, POU4F2, and ZNF154. In some embodiments, (a) the cancer is bladder cancer, and the one or more target genomic regions comprise one or more target sequences of one or more genes selected from Tables 2 or 3: (b) the cancer is prostate cancer, and the one or more target genomic regions comprise one or more target sequences of one or more genes selected from Table 4: or (c) the cancer is kidney cancer, and the one or more target genomic regions comprise one or more target sequences of one or more genes selected from Table 5. In some embodiments, each target genomic region comprises at least five methylation sites.

In one aspect, provided herein are compositions comprising a plurality of different bait oligonucleotides. In some embodiments. (a) the bait oligonucleotides hybridize to converted DNA molecules derived from one or more target genomic regions: (b) the one or more target genomic regions comprise one or more target sequences of one or more genes selected from Table 1: (c) the one or more target genomic regions are differentially methylated in a cancer; and (d) each bait oligonucleotide comprises a sequence at least 25 nucleotides in length that hybridizes to one of the target sequences. In some embodiments, the one or more target genomic regions comprise target sequences from at least 10 genes selected from Table 1. In some embodiments, the one or more target genomic regions comprise a target sequence of gene selected from TWIST1, EOMES, HOXA9, POU4F2, and ZNF154. In some embodiments, the one or more target genomic region comprise target sequences from: (a) genes in Tables 2 or 3; (b) genes in Table 4: or (c) genes in Table 5. In some embodiments, each target genomic region comprises at least five methylation sites. In some embodiments, the differential methylation comprises at least 80% of CpG sites in the target genomic region being methylated or unmethylated.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a workflow for urine sample processing, according to one embodiment.

FIG. 2A illustrates an exemplary process for biopsy-free tumor fraction estimation from urine cell-free DNA. FIG. 2B illustrates an exemplary methylation variant at CpG 1528723-1528728. The methylation variant is illustrated as a stretch of contiguous CpG and methylation states that distinguishes cancer-derived cfDNA from non-cancer cfDNA.

FIG. 3 provides data showing that urological cancers (bladder or urothelial, kidney, and prostate cancers) had lower detection sensitivity in a Circulating Cell-free Genome Atlas Study (CCGA) patient cohort. Each pair of bars represents results for early stage (left) and late stage (right).

FIG. 4 illustrates a process for methylation variant determination from urine cfDNA, according to one embodiment.

FIG. 5A provides data illustrating cfDNA fragment size distribution and yield when processed by adding preservative to the sample immediately following collection. FIG. 5B provides data illustrating the effect of a one-hour delay in preservative addition to urine samples on cfDNA fragment size distribution and yield.

FIGS. 6A-6B provide data on the cancer-specific methylation signatures of four genes detected in urine cfDNA from a subject with stage I, high-grade non-muscle invasive bladder cancer pre- and post-surgery.

FIG. 7 provides data comparing the estimated tumor fraction in urine and plasma samples from subjects with (plotted points with annotations) or without (plotted points without annotations) bladder cancer of any stage.

FIG. 8 provides data comparing the estimated tumor fraction in urine and plasma samples from subjects with or without stage III or IV prostate cancer, Data points from the “*” and above correspond to results for samples only from subjects with cancer, Data points below the “*” correspond to results for samples from cancer and non-cancer subjects.

FIG. 9 provides data comparing the estimated tumor fraction in urine and plasma samples from subjects with (data points with annotations) or without (data points without annotations) kidney cancer,

FIG. 10A provides data on the classification performance for urine cfDNA from subjects with bladder cancer based on a subset of 15 genomic region, showing an area under the curve (AUC) of 0.99. Two of the genomic regions are represented by either of two genes within the region. FIG. 10B provides data on the classification performance for urine cfDNA from subjects with bladder cancer based on genomic regions within a single gene (TWIST1), showing an AUC of 0.86. The shading represents the 95% confidence interval. FIG. 10C provides data on the classification performance for urine cfDNA from subjects with kidney cancer based on a subset of genomic regions, showing an AUC of 0.52. The shading represents the 95% confidence interval. FIG. 10D provides data on the classification performance for urine cfDNA from subjects with prostate cancer based on a subset of genomic regions, showing an AUC of 0.82. The shading represents the 95% confidence interval.

FIG. 11A illustrates a 2× tiled probe design, with three probes targeting a small target region, where each base in a target region (boxed in the dotted rectangle) is covered by at least two probes, according to an embodiment. FIG. 11B illustrates a 2× tiled probe design, with more than three probes targeting a larger target region, where each base in a target region (boxed in the dotted rectangle) is covered by at least two probes, according to an embodiment. FIG. 11C illustrates probe design targeting hypomethylated and/or hypermethylated fragments in genomic regions, according to an embodiment. FIG. 11D illustrates a section of a genome comprising three target genomic regions. Probes are designed to hybridize to each target and its adjacent sequences in a 2× tiled configuration, according to an embodiment. FIG. 11E illustrates a single pair of probes that hybridize within the same target genomic regions, each comprising an overlapping sequence and a non-overlapping sequence. The overlapping sequences are complementary to the same target sequence. The non-overlapping sequences are complementary to different sequences within the target genomic region, each at a different end of the sequence to which the overlapping sequence is complementary, as shown.

FIG. 12A is a flowchart describing a process of creating a data structure for a control group, according to an embodiment. FIG. 12B is a flowchart describing an additional step of validating the data structure for the control group of FIG. 12A, according to an embodiment.

FIG. 13 is a flowchart describing a process for selecting genomic regions for designing probes for a cancer assay panel, according to an embodiment.

FIG. 14 is an illustration of an example p-value score calculation, according to an embodiment.

FIG. 15A is a flowchart describing a process of training a classifier based on hypomethylated and hypermethylated fragments indicative of cancer, according to an embodiment. FIG. 15B is a flowchart describing a process of identifying fragments indicative of cancer determined by probabilistic models, according to an embodiment.

FIG. 16A is a flowchart describing a process of sequencing a fragment of cell-free (cf) DNA, according to an embodiment. FIG. 16B is an illustration of a process of sequencing a fragment of cell-free (cf) DNA to obtain a methylation state vector, according to an embodiment.

FIG. 17A illustrates a flowchart of devices for sequencing nucleic acid samples according to one embodiment. FIG. 17B illustrates an analytic system that analyzes methylation status of cfDNA according to one embodiment.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise herein, all technical and scientific terms used herein have the same meaning as commonly understood by one of ordinary skill in the art to which this disclosure belongs. The following definitions are provided to facilitate understanding of certain terms used frequently herein.

Where a range of values is provided, it is understood that each intervening value, to the tenth of the unit of the lower limit, unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “methylation” as used herein refers to a process by which a methyl group is added to a DNA molecule. For example, a hydrogen atom on the pyrimidine ring of a cytosine base can be converted to a methyl group, forming 5-methylcytosine. The term also refers to a process by which a hydroxymethyl group is added to a DNA molecule, for example by oxidation of a methyl group on the pyrimidine ring of a cytosine base. Methylation and hydroxymethylation tend to occur at dinucleotides of cytosine and guanine referred to herein as “CpG sites.” The principles described herein are also applicable for the detection of methylation in a non-CpG context, including non-cytosine methylation. In such embodiments, a wet laboratory assay used to detect methylation may vary from any described herein. Further, the methylation state vectors may contain elements that are generally vectors of sites where methylation has or has not occurred (even if those sites are not CpG sites specifically).

The term “methylation” can also refer to the methylation status of a CpG site. A CpG site with a 5-methylcytosine moiety is methylated. A CpG site with a hydrogen atom on the pyrimidine ring of the cytosine base is unmethylated.

The term “methylation site” as used herein refers to a region of a DNA molecule where a methyl group can be added. “CpG” sites are the most common methylation site, but methylation sites are not limited to CpG sites. For example, DNA methylation may occur in cytosines in CHG and CHH, where H is adenine, cytosine or thymine. Cytosine methylation in the form of 5-hydroxymethylcytosine may also assessed (see, e.g., US20110236894A1 and US20110301045A1, which are incorporated herein by reference), and features thereof, using the methods and procedures disclosed herein.

The term “CpG site” as used herein refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group. Cytosines in CpG dinucleotides can be methylated to form 5-methylcytosine.

In some embodiments, oligonucleotide probes described herein comprise one or more CpG detection sites. The term “CpG detection site” as used herein refers to a region in a probe that is configured to hybridize to a CpG site of a target DNA molecule. The CpG site on the target DNA molecule can comprise cytosine and guanine separated by one phosphate group, where cytosine is methylated or unmethylated. The CpG site on the target DNA molecule can comprise uracil and guanine separated by one phosphate group, where the uracil is generated by the conversion of unmethylated cytosine.

The term “UpG” is a shorthand for 5′-U-phosphate-G-3′ that is uracil and guanine separated by only one phosphate group. UpG can be generated by a bisulfite treatment of a DNA that converts unmethylated cytosines to uracils. Cytosines can be converted to uracils by other methods such as chemical modification, synthesis, or enzymatic conversion.

The terms “hypomethylated” or “hypermethylated” as used herein refer to a methylation status of a DNA molecule containing multiple CpG sites (e.g., more than 2, 3, 4, 5, 6, 7, 8, 9, 10, etc.) where a high percentage of the CpG sites (e.g., more than 80%, 85%, 90%, or 95%, or any other percentage within the range of 50%-100%, 70% or more, 75% or more, 80% or more, 85% or more, 90% or more, 95% or more, 97.5% or more, 98% or more, 99% or more, 99.9% or more, or any other numerical percentage within the range of 50%-100% or more, wherein the provided range includes the range limits of 50% and 100%) are unmethylated or methylated, respectively. For example, “hypomethylated” nucleic acid, e.g., cfDNA, fragments can be fragments having a number, e.g., 3 or more, 4 or more, 5 or more, 6, or more, 7 or more, 9 or more, 10 or more, of CpG sites with a percentage, e.g., 70% or more, 75% or more, 80% or more, 85% or more, 90% or more, or 95% or more, or 97.5% or more, 98% or more, 99% or more. 99.9% or more, of the CpG sites being unmethylated. Likewise, “hypermethylated” nucleic acid, e.g., cfDNA, fragments can be fragments having a number, e.g., 3 or more, 4 or more, 5 or more, 6 or more, 7 or more, 9 or more, 10 or more, of CpG sites with a percentage, e.g., 70% or more, 75% or more, 80% or more, 85% or more, 90% or more, or 95% or more, or 97.5% or more, 98% or more, 99% or more, 99.9% or more of the CpG sites being methylated. In some embodiments, a hypomethylated DNA molecule comprises a plurality of CpG sites, at least 80% of which are unmethylated. In some embodiments, a hypermethylated DNA molecule comprises a plurality of CpG sites, at least 80% of which are methylated.

The terms “methylation state vector” or “methylation status vector” as used herein refer to a vector comprising multiple elements, where each element indicates the methylation status of a methylation site in a DNA molecule comprising multiple methylation sites, in the order they appear from 5′ to 3′ in the DNA molecule. For example, <Mₓ, Mₓ₊₁, Mₓ₊₂>, <Mₓ, Mₓ₊₁, Uₓ₊₂>, . . . , <Uₓ, Uₓ₊₁, Uₓ₊₂> can be methylation vectors for DNA molecules comprising three methylation sites, where M represents a methylated methylation site and U represents an unmethylated methylation site.

The terms “abnormal methylation pattern” and “anomalous methylation pattern” as used herein refer to a methylation pattern of a nucleic acid, e.g., DNA such as a cfDNA, molecule or a methylation state vector that is found and/or expected to be found in a sample less frequently than it would be in a healthy, e.g., non-cancer, sample. In various embodiments, such a methylation pattern is found and/or expected to be found in a sample with a lower frequency than a threshold value of a healthy, e.g., non-cancer, sample. As such, for example, the terms “abnormally methylated” and “anomalously methylated” as used herein describe a nucleic acid. e.g., DNA such as a cfDNA, molecule or a methylation state vector exhibiting an abnormal methylation pattern. An aspect according to the subject disclosure that is differentially methylated can in some versions include an aspect that is abnormally methylated. Also, whether an aspect is differentially methylated can be used as an indicator for a determination of healthy, e.g., non-cancer, as opposed to diseased, e.g., cancer, in referring to the health of a subject from which a subject sample was originated. In one embodiment provided herein, the finding and/or expectedness of finding a specific methylation state vector in a healthy control group including healthy individuals is represented by a p-value. In various aspects, a low p-value score corresponds to a methylation state vector which is relatively unexpected in comparison to other methylation state vectors within samples from healthy individuals, such as individuals in a healthy control group. In some versions, a high p-value score corresponds to a methylation state vector which is relatively more expected in comparison to other methylation state vectors found in samples from healthy individuals such as those in the healthy control group. In various embodiments, a methylation state vector having an abnormal/anomalous methylation pattern is a methylation state vector having a p-value at and/or lower than a threshold value (e.g., 0.1, 0.01, 0.001, 0.0001, etc.), such as a threshold value corresponding with a healthy, e.g., non-cancer, sample. In various embodiments, the methods include associating a methylation state vector from a sample and having a p-value at and/or lower than a threshold value (e.g., 0.1 or smaller. 0.01 or smaller, 0.001 or smaller. 0.0001 or smaller, etc.) with a determination that the sample is not a healthy sample, e.g., is a sample from a subject having cancer. In various embodiments, the threshold value is applied as a filter in that application of a smaller threshold value (e.g., 0.001, 0.0001, etc.) is associated with a higher expectation that a methylation state vector is from a sample that is not healthy, e.g., is a sample from an individual with cancer, Various methods can be used to calculate a p-value or expectedness of a methylation pattern or a methylation state vector. Exemplary methods provided herein involve use of a Markov chain probability that assumes methylation statuses of CpG sites to be dependent on methylation statuses of neighboring CpG sites. Alternate methods provided herein calculate the expectedness of observing a specific methylation state vector in healthy individuals by utilizing a mixture model including multiple mixture components, each being an independent-sites model where methylation at each CpG site is assumed to be independent of methylation statuses at other CpG sites. In some versions, the subject methods include determining whether a nucleic acid, e.g., DNA, molecule or a methylation state vector is abnormally methylated. In various embodiments of the methods, a generated p-value is compared, such as by an analytics system, against a threshold to identify vectors, e.g., nucleic acids such as cfDNA fragments, that are abnormally methylated relative to a control group, such as a group associated with one or more healthy, e.g., non-cancer, samples. In addition, abnormal methylation, e.g., cfDNA methylation, can be hypermethylation and/or hypomethylation, both of which can be indicative of a non-healthy. e.g., cancer, status. Accordingly, the methods include determining healthy or diseased, e.g., non-cancer or cancer, status based at least in part on a p-value, such as a relatively low p-value, such as a p-value below a threshold, wherein the p-value can in various aspects be indicative of abnormal methylation, e.g., hypermethylation and/or hypomethylation. A low p-value, e.g., a p-value at or below a threshold value (e.g., 0.1, 0.01, 0.001, 0.0001, etc.), can be indicative of abnormal methylation, e.g., hypermethylation and/or hypomethylation in a sample. In various embodiments, the methods include determining healthy or diseased, e.g., non-cancer or cancer, status of a sample based on a nucleic acid, e.g., nucleic acid fragment, or methylation vector from the sample having a low p-value, e.g., equal to or less than 0.1, 0.01 or 0.001, and being both hypermethylated and hypomethylated or hypermethylated or hypomethylated. In various aspects, the methods include determining healthy or diseased, e.g., non-cancer or cancer, status of a sample based at least in part on whether a nucleic acid, e.g., nucleic acid fragment, or methylation vector from the sample, is both hypermethylated and hypomethylated. In some variations, determining a vector, e.g., sample fragment, to be anomalously methylated based on a generated p-value score includes determining whether the generated score for the vector is below a threshold score, wherein the threshold score is a degree of confidence that the vector is anomalously methylated.

The term “cancerous sample” as used herein refers to a sample comprising genomic DNAs from an individual diagnosed with cancer. The genomic DNAs can be, but are not limited to, cfDNA fragments or chromosomal DNAs from a subject with cancer. The genomic DNAs can be sequenced and their methylation status can be assessed by various methods, for example, bisulfite sequencing. When genomic sequences are obtained from public database (e.g., The Cancer Genome Atlas (TCGA)) or experimentally obtained by sequencing a genome of an individual diagnosed with cancer, cancerous sample can refer to genomic DNAs or cfDNA fragments having the genomic sequences. The term “cancerous samples” as a plural refers to samples comprising genomic DNAs from multiple individuals, each individual diagnosed with cancer. In various embodiments, cancerous samples from more than 100, 300, 500, 1,000, 2,000, 5,000, 10,000, 20,000, 40,000, 50.000, or more individuals diagnosed with cancer are used.

The term “non-cancerous sample” or “healthy sample” as used herein refers to a sample comprising genomic DNAs from a healthy individual or an individual not diagnosed with cancer. The genomic DNAs can be, but are not limited to, cfDNA fragments or chromosomal DNAs from a subject without cancer. The genomic DNAs can be sequenced and their methylation status can be assessed by various methods, for example, bisulfite sequencing. When genomic sequences are obtained from public database (e.g., The Cancer Genome Atlas (TCGA)) or experimentally obtained by sequencing a genome of an individual without cancer, non-cancerous sample can refer to genomic DNAs or cfDNA fragments having the genomic sequences. The term “non-cancerous samples” as a plural refers to samples comprising genomic DNAs from multiple individuals, each individual is without cancer. In various embodiments, cancerous samples from more than 100, 300, 500, 1,000, 2,000, 5,000, 10,000, 20,000, 40,000, 50,000, or more individuals without cancer are used. In various embodiments, cancerous samples from 100 or more, 300 or more, 500 or more, 1,000 or more, 2,000 or more, 5,000 or more, 10,000 or more, 20,000 or more, 40,000 or more, or 50,000 or more individuals without cancer are used.

The term “training sample” as used herein refers to a sample used to train a classifier described herein and/or to select one or more genomic regions for cancer detection or detecting a cancer tissue of origin or cancer cell type. The training samples can comprise genomic DNAs or a modification there of, from one or more healthy subjects and from one or more subjects having a disease condition (e.g., cancer, a specific type of cancer, a specific stage of cancer, etc.). The genomic DNAs can be, but are not limited to, cfDNA fragments or chromosomal DNAs. The genomic DNAs can be sequenced and their methylation status can be assessed by various methods, for example, bisulfite sequencing. When genomic sequences are obtained from a public database (e.g., The Cancer Genome Atlas (TCGA)) or experimentally obtained by sequencing a genome of an individual, a training sample can refer to genomic DNAs or cfDNA fragments having the genomic sequences.

The term “test sample” as used herein refers to a sample from a subject, whose health condition was, has been or will be tested using a classifier and/or an assay panel described herein. The test sample can comprise genomic DNAs or a modification thereof. The genomic DNAs can be, but are not limited to, cfDNA fragments or chromosomal DNAs.

The term “target genomic region” as used herein refers to a region in a genome selected for analysis in test samples. An assay panel is generated with oligonucleotide probes designed to hybridize to (and optionally pull down) nucleic acid fragments derived from the target genomic region or a fragment thereof. Oligonucleotide probes directed to target regions are also referred to herein as “bait oligonucleotides.” A nucleic acid fragment derived from the target genomic region refers to a nucleic acid fragment generated by degradation, cleavage, bisulfite conversion, or other processing of the DNA from the target genomic region. In some embodiments, a plurality of different bait oligonucleotides are designed to hybridize across a single target genomic region (e.g., overlapping probes tiled across a target genomic region). In general, when referring to a plurality of target genomic regions, no target genomic region of the plurality is wholly contained within another target genomic region. Different target genomic regions in a plurality of target genomic regions may be overlapping, but will at least have different termini. In some embodiments, each target genomic region in a plurality of target genomic regions is separate from and does not overlap with any other target genomic region in the plurality. In some embodiments, a target genomic region comprises a target sequence of a gene. In this context, the term “gene” encompasses sequences encoding the gene, and optionally flanking sequences (e.g., regulatory sequences, such as promoter, enhancer, and untranslated regions), and internal non-coding sequences (e.g., introns). In some embodiments, a gene is defined by the sequence encoding the transcription start and stop sites, and an additional 5000 nucleotides from each of these ends. A given gene may comprise multiple target sequences.

Target genomic regions may be described according to their chromosomal location, such as with regard to a particular reference sequence (e.g., the human reference genome GRCh37/hg19). Chromosomal DNA is double-stranded, so a target genomic region includes two DNA strands: one with the sequence according to a given reference sequence, and a second that is a reverse complement thereof. Probes can be designed to hybridize to one or both sequences. Optionally, probes hybridize to converted sequences resulting from, for example, treatment with sodium bisulfite.

The term “off-target genomic region” as used herein refers to a region in a genome which has not been selected for analysis in test samples but has sufficient homology to a target genomic region to potentially be bound and pulled down by a probe designed to target the target genomic region. In one embodiment, an off-target genomic region is a genomic region that aligns to a probe along at least 45 bp with at least a 90% match rate.

The terms “converted DNA molecules.” “converted cfDNA molecules.” and “modified fragment obtained from processing of the cfDNA molecules” refer to DNA molecules obtained by processing DNA or cfDNA molecules in a sample for the purpose of differentiating a methylated nucleotide and an unmethylated nucleotide in the DNA or cfDNA molecules. For example, in some embodiments, the sample can be treated with bisulfite ion (e.g., using sodium bisulfite), to convert unmethylated cytosines (“C”) to uracils (“U”). In another embodiment, the conversion of unmethylated cytosines to uracils is accomplished using an enzymatic conversion reaction, for example, using a cytidine deaminase (such as APOBEC). After treatment, converted DNA molecules or cfDNA molecules include additional uracils which are not present in the original cfDNA sample, Replication by DNA polymerase of a DNA strand comprising a uracil results in addition of an adenine to the nascent complementary strand instead of the guanine normally added as the complement to a cytosine or methylcytosine.

In general, the terms “cell-free.” “circulating.” and “extracellular” as applied to polynucleotides (e.g. “cell-free DNA” or “cfDNA”) are used interchangeably to refer to polynucleotides present in a sample from a subject or portion thereof that can be isolated or otherwise manipulated without applying a lysis step to the sample as originally collected (e.g., as in lysis for the extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g, apoptosis or necrosis) or cell shedding, releasing polynucleotides into surrounding body fluids or into circulation. Accordingly, cell-free nucleic acids may be isolated from a non-cellular fraction of blood (e.g. serum or plasma), from other bodily fluids (e.g. urine), or from non-cellular fractions of other types of samples. Non-limiting examples of body fluids that may be used in conjunction with embodiments disclosed herein include mucous, blood, plasma, serum, serum derivatives, synovial fluid, lymphatic fluid, bile, phlegm, saliva, sweat, tears, sputum, amniotic fluid, menstrual fluid, vaginal fluid, semen, urine, cerebrospinal fluid (CSF), such as lumbar or ventricular CSF, gastric fluid, a liquid sample comprising one or more material(s) derived from a nasal, throat, or buccal swab, a liquid sample comprising one or more materials derived from a lavage procedure, such as a peritoneal, gastric, thoracic, or ductal lavage procedure, and the like. In some embodiments, cfDNA refers to deoxyribonucleic acid molecules that circulate in a subject's body (e.g., bloodstream) and may originate from one or more healthy cells and/or from one or more cancer cells. In some embodiments, cfDNA is cfDNA of a urine sample. In some embodiments, compositions (e.g., an assay panel) and methods disclosed herein in connection with urine cfDNA may be applied or adapted for use with other sample types (e.g., cfDNA from other bodily fluids, such as blood, serum, or plasma).

The term “circulating tumor DNA” or “ctDNA” refers to nucleic acid fragments that originate from tumor cells, which may be released into an individual's bloodstream as result of biological processes such as apoptosis or necrosis of dying cells or actively released by viable tumor cells.

The term “fragment” as used herein can refer to a fragment of a nucleic acid molecule. For example, in one embodiment, a fragment can refer to a cfDNA molecule in a blood or plasma sample, or a cfDNA molecule that has been extracted from a blood or plasma sample, An amplification product of a cfDNA molecule may also be referred to as a “fragment.” In another embodiment, the term “fragment” refers to a sequence read, or set of sequence reads, that have been processed for subsequent analysis (e.g., for machine-learning based classification), as described herein. For example, raw sequence reads can be aligned to a reference genome and matching paired end sequence reads assembled into a longer fragment for subsequent analysis.

The terms “individual” and “subject” refer to a human individual. The term “healthy individual” refers to an individual presumed not to have a cancer or disease. In some embodiments, a subject is an individual whose DNA is being analyzed. For example, a subject may be a test subject whose DNA is be evaluated using a targeted panel as described herein to evaluate whether the person has cancer or another disease. In some embodiments, a subject is part of a control group known to have (or not have) cancer or another disease (also referred to as a “reference subject”). Control and cancer/disease groups may be used to assist in designing or validating the targeted panel.

The term “sequence read” as used herein refers to a string of nucleotides as determined for part of, or all of, a nucleic acid molecule by a nucleic acid sequencing process. A sequence read may be a short string of nucleotides (e.g., 20-150) sequenced from a nucleic acid fragment, a short string of nucleotides at one or both ends of a nucleic acid fragment, or the sequencing of the entire nucleic acid fragment that exists in the biological sample, Sequence reads can be obtained through various methods provided herein or by other methods known in the art.

The term “sequencing depth” as used herein refers to the count of the number of times a given target nucleic acid within a sample has been sequenced (e.g., the count of sequence reads at a given target region), or is on average actually or expected to be sequenced based on the amount of nucleic acid subjected to sequencing and the total read length generated by a given sequencing process (e.g., an average of the read depth for all sequenced regions from a given sequencing run). Increasing sequencing depth can reduce required amounts of nucleic acids required to assess a disease state (e.g., cancer or cancer tissue of origin).

The term “tissue of origin” or “TOO” as used herein refers to the organ, organ group, body region or cell type that a cancer arises or originates from. The identification of a tissue of origin or cancer cell type typically allows for identification of the most appropriate next steps in the care continuum of cancer to further diagnose, stage and decide on treatment. For example, a cancer that originates from a cell in the bladder may be identifiable as a bladder cell based on one or more markers associated with bladder cells, even after metastasizing to a different tissue, such as a kidney. In the case of metastasis, the “cancer tissue” refers to the metastatic growth of cells of the particular TOO, distinct from the cells of the tissue to which the cancer has metastasized. Thus, a metastatic cancer tissue may be located adjacent or within healthy tissue at the metastatic site.

“Treating” or “treatment” as used herein includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words. “treatment” as used herein includes any cure, amelioration, or prevention of a disease. Treatment may prevent the disease from occurring: inhibit the disease's spread: relieve the disease's symptoms, fully or partially remove the disease's underlying cause, shorten a disease's duration, or do a combination of these things.

“Treating” and “treatment” as used herein includes prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent”, as pertains to a disease or condition of a subject, refers to a decrease in the occurrence of one or more corresponding symptoms in the subject. As indicated above, the prevention may be complete (no detectable symptoms) or partial, such that fewer symptoms are observed, and/or with lower incidence, than would likely occur absent treatment.

“Anti-cancer agent” and “anticancer agent” are used in accordance with their plain ordinary meanings and refer to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer, Examples of anti-cancer agents include, but are not limited to, MEK (e.g, MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126. PD98059. PD184352. PD0325901. ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412. PD184352. 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleuki II, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to 111In, 90Y, or 1311, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™) erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

In some embodiments, the anti-cancer agent is an epigenetic inhibitor. An “epigenetic inhibitor” as used herein, refers to an inhibitor of an epigenetic process, such as DNA methylation (a DNA methylation Inhibitor) or modification of histones (a Histone Modification Inhibitor). An epigenetic inhibitor may be a histone-deacetylase (HDAC) inhibitor, a DNA methyltransferase (DNMT) inhibitor, a histone methyltransferase (HMT) inhibitor, a histone demethylase (HDM) inhibitor, or a histone acetyltransferase (HAT). Examples of HDAC inhibitors include Vorinostat, romidepsin, CI-994, Belinostat, Panobinostat, Givinostat, Entinostat, Mocetinostat, SRT501, CUDC-101, JNJ-26481585, or PCI24781. Examples of DNMT inhibitors include azacitidine and decitabine. Examples of HMT inhibitors include EPZ-5676. Examples of HDM inhibitors include pargyline and tranylcypromine. Examples of HAT inhibitors include CCT077791 and garcinol.

In some embodiments, the anti-cancer agent is a multi-kinase inhibitor. A “multi-kinase inhibitor” is a small molecule inhibitor of at least one protein kinase, including tyrosine protein kinases and serine/threonine kinases. A multi-kinase inhibitor may include a single kinase inhibitor. Multi-kinase inhibitors may block phosphorylation. Multi-kinases inhibitors may act as covalent modifiers of protein kinases. Multi-kinase inhibitors may bind to the kinase active site or to a secondary or tertiary site inhibiting protein kinase activity. A multi-kinase inhibitor may be an anti-cancer multi-kinase inhibitor. Exemplary anti-cancer multi-kinase inhibitors include dasatinib, sunitinib, erlotinib, bevacizumab, vatalanib, vemurafenib, vandetanib, cabozantinib, poatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib.

### Urine Sample Assays

In one aspect, the present disclosure provides methods of sequencing cell-free nucleic acid molecules of a subject. In some embodiments, the method comprises (a) treating a urine sample to inhibit cell lysis; (b) separating cell-free nucleic acid molecules in the treated urine sample from cells in the treated urine sample, thereby producing a purified urine sample comprising the cell-free nucleic acid molecules; (c) concentrating the cell-free nucleic acid molecules in the purified urine sample by passing at least a portion of the purified urine sample through a filter, wherein (i) the concentrating producing a filtrate and a retained urine sample, and (ii) the retained urine sample comprises an increased concentration of cell-free nucleic acid molecules; (d) isolating cell-free nucleic acid molecules from the retained urine sample; and (e) sequencing the isolated cell-free nucleic acid molecules.

Urine samples for use in accordance with the present disclosure may be collected from a variety of sources and exhibit various characteristics. In some embodiments, the urine sample comprises a desired minimum initial volume. For example, the urine sample may have a volume of at least 5 mL, 10 mL, 20 mL, 30 mL, 40 mL, 50 mL, 75 mL, 100 mL, or more. In some embodiments, the volume of the treated urine sample is 5 mL, 10 mL, 15 mL, 20 mL, 30 mL, 40 mL, 50 mL, or more. In some embodiments, the urine sample is a sample of at least 50 mL.

Typically, a urine sample as collected will comprise cell-free nucleic acid molecules and other components, such as cells, metabolites, proteins, and salts. In some embodiments, the urine sample is treated to inhibit cell lysis. Inhibition of cell lysis need not be absolute, but will in general reduce the rate of cell lysis in the sample, as compared to an untreated urine sample. In some embodiments, treating the urine sample to inhibit cell lysis comprises contacting the urine sample with a one or more preservative reagent. A variety of suitable preservative reagents are available. Preservative reagents that can be used to inhibit cell lysis include, but are not limited to, imidazolidinyl urea (IDU), diazolidinyl urea (DU), dimethylol urea, 2-bromo-2-nitropropane-1,3-diol, 5-hydroxymethoxymethyl-1-aza-3,7-dioxabicyclo (3.3.0)octane and 5-hydroxymethyl-1-aza-3,7-dioxabicyclo (3.3.0)octane and 5-hydroxypoly [methyleneoxy]methyl-1-aza-3,7-dioxabicyclo (3.3.0)octane, bicyclic oxazolidines (e.g. Nuosept 95), DMDM hydantoin, sodium hydroxymethylglycinate, hexamethylenetetramine chloroallyl chloride (Quatemium-15), biocides (such as Bioban, Preventol and Grotan), a water-soluble zinc salt, sodium azide, or any combination thereof. In some embodiments, the preservative reagent comprises imidazolidinyl urea.

In some embodiments, treating a urine sample to inhibit cell lysis comprises treatment with a combination of reagents, such as treatment with a preservative reagent, a nuclease inhibitor, a formaldehyde quencher, or a combination of these. Treatment of the urine sample to inhibit cell lysis may affect the integrity of the nucleic acids in the samples. Some cell lysis inhibitors can release formaldehyde, which can compromise or destroy the structural integrity of nucleic acids if not quenched. A formaldehyde quencher may thus be added to preserve stability of the nucleic acids in the sample. Formaldehyde quenchers that may be used include, but are not limited to glycine, Tris(hydroxymethyl)aminomethane (TRIS), urea, allantoin, sulfites or any combination thereof. In some embodiments, the formaldehyde inhibitor is glycine.

In some embodiments, treating the urine sample includes treatment with a nuclease inhibitor. Nuclease activity in fresh urine can rapidly hydrolyze nucleic acids (e.g., DNA). Nuclease inhibitors that may be used include, but are not limited to, ethylene glycol tetraacetic acid (EGTA), pepstatin, EDTA, phosphoramidon, leupeptin, aprotinin, bestatin, proteinase inhibitor E 64 (E-64), 4-(2-Aminoethyl) benzenesulfonyl fluoride hydrochloride (AEBSF), or any combination thereof. In some embodiments, the nuclease inhibitor is EDTA. Treatment with the nuclease inhibitor may also be performed with or without including a formaldehyde quencher. In some embodiments, the treatment includes a nuclease inhibitor and no formaldehyde quencher. In some embodiments, the treatment includes a nuclease inhibitor and a formaldehyde quencher.

In some embodiments, treating comprises contacting the urine sample with a composition comprising a cell lysis inhibitor, and a nuclease inhibitor, a formaldehyde quencher, or both. In some embodiments, the composition comprises imidazolidinyl urea, EDTA, glycine, or a combination thereof. In some embodiments, imidazolidinyl urea is added to a final concentration of 0.5% to 2.0%. In some embodiments, imidazolidinyl urea is added to a final concentration of 0.20% to 4.0%. In some embodiments, the glycine is present at a concentration of 0.01% to 0.2%. In some embodiments, the glycine is present at a concentration of 0.01% to 0.4%. In some embodiments, the glycine is present at a concentration of at least 0.01%, at least 0.05%, at least 0.2%, or even at least 0.3%. In some embodiments, the EDTA is present at a concentration of 0.5% to 2.0%. In some embodiments, the EDTA is present at a concentration of 0.50% to 3.6%. In some embodiments, the EDTA is present at a concentration of at least 0.5%, at least 1%, or at least 2.5%. In some embodiments, the ratio of EDTA to imidazolidinyl urea is 5:2. In some embodiments, the ratio of EDTA to imidazolidinyl urea is from 1:3 to 3:1 (e.g., 9:10, 9:5 or 9:20). In some embodiments, the ratio of EDTA to glycine is from 5:1 to 100:1 (e.g., 50:1 or 9:1).

In some embodiments, the composition used in treating the urine sample to inhibit cell lysis comprises sodium azide, EDTA, or a combination thereof. In some embodiments, the sodium azide is present at a final concentration of 0.05% to 2.0%. In some embodiments, the sodium azide is present at a concentration of 0.10% to 1.0%. In some embodiments, the EDTA is present at a concentration of 0.5% to 2.0%. In some embodiments, the EDTA is present at a concentration of 0.50% to 3.6%. In some embodiments, the EDTA is present at a concentration of at least 0.5%, at least 1%, or at least 2.5%.

In some embodiments, the composition used in treating the urine sample to inhibit cell lysis is present in an amount of 1 to 20 percent by volume of the urine sample after contacting. The preservative reagent may be combined with the urine sample following collection (e.g., by adding to the urine sample, or transferring all or a portion of the urine sample to a container with the preservative reagent), or may be present in the container used to collect the sample. Additional non-limiting examples of compositions comprising one or more of a preservative reagent, a nuclease inhibitor, or formaldehyde quencher are described in U.S. Publication No. 20160257995 A1, which is incorporated herein by reference.

In some embodiments, the method includes a step of separating cell-free nucleic acid molecules (e.g., cfDNA and/or cfRNA) in the treated urine sample from cells in the treated urine sample, in order to produce a purified urine sample comprising the cell-free nucleic acid molecules (and reduced in cellular content). A variety of methods for separating cell-free nucleic acids from cells are available, non-limiting examples of which include fractionation, centrifugation (e.g., pelleting, or density gradient centrifugation), precipitation, and flow cytometry. In some embodiments, separating comprises centrifugation of a treated urine sample to pellet cells. The cell pellet may then be removed or the supernatant transferred to a new container. In some embodiments, beginning with the treated urine sample, cell-free nucleic acids (e.g., cell-free DNA) may be separated from cells by centrifugation, e.g., at 3000 rpm to 8000 rpm for 10 to 15 minutes at room temperature. The treated urine sample may be centrifuged at at least 1000 g, 2000 g, 3000 g, 4000 g, 5000 g, 6000 g, 7000 g, 8000 g or more. Additionally, centrifugation may be performed for at least 5, 10, 15, 20, 30, 45, 60, or more minutes. In some embodiments, the treated urine sample is centrifuged at 4000 g for 20 minutes. Separated cells will form a pellet of cells upon centrifugation, which can be removed by transferring the supernatant to a different container, thus separating the cells from the nucleic acids. Separation can be performed one, two, three, four, or more times, or until no more visible cell pellet forms upon centrifugation.

In some embodiments, the purified urine sample is subjected to a concentrating step to produce a sample with an increased concentration of nucleic acids. Concentration may provide certain advantages, such as working with larger input sample volumes and compensating for reduced concentration of cell-free nucleic acids in urine as compared to other sample types (e.g., plasma). Sample with a higher concentration of nucleic acids and lower volume may utilize reduced quantities of reagent and materials for processing and allow for easier handling of multiple samples in parallel. In some embodiments, concentration of the cell-free nucleic acid molecules in the purified urine sample is performed by passing at least a portion of the purified urine sample through a filter. Passing the purified urine sample through the filter produces a filtrate and a retained urine sample. The filtrate will contain salts and other small components that can pass through the filter, while the retained urine sample will contain the nucleic acids at an increased concentration. In some instances, the filter is substantially impermeable to nucleic acids (e.g., cell-free nucleic acids), and substantially permeable to salts and other smaller components in the purified urine sample. When filtering a large sample volume in two or more portions, each of the two or more portions may be applied serially to the same filter, separately to different filters, or some combination of these. A retained portion of a filtered sample may be subjected to one or more additional rounds of filtering (e.g., 2, 5, 10, 15, or more rounds), such as by repeated applications on the same filter, or serial application to different filters.

Any of a variety of types of filters made of various materials may be used, and several options are commercially available. For instance, the filter may be a membrane filter, such as those made of nylon, cellulose, or nitrocellulose, or include beads (e.g., sepharose beads). In some embodiments, the filter is characterized by a molecular cutoff. In general, a molecular weight cutoff results from a particular pore size and/or coating that allows separation of molecules above the cutoff from molecules below the cutoff. Molecular weight cutoffs (also referred to as “molecular weight limits”) are typically specified as among the characteristics of commercially available filters for processing biological samples. In some embodiments, the molecular weight cutoff represents the molecular weight of a solute that is 90% retained by the filter. In some embodiments, the filter comprises a rated molecular weight cutoff of 1 kilodalton (kD) to 50 kD. In some embodiments the filter comprises a rated molecular wight cutoff of 3 kD to 10 kD. In some embodiments the filter comprises a rated molecular wight cutoff of 10 kD, 5 kD, 3 kD, or lower. In some embodiments the filter comprises a rated molecular wight cutoff of 3 kD or lower.

Various concentrations of nucleic acids in the retained urine sample relative to the purified urine sample may be achieved, following one or more concentration steps. In some embodiments, the retained urine sample has a concentration that is increased by at least 2-fold compared to the purified urine sample (prior to concentrating). In some embodiments, the retained urine sample has a concentration that is increased by at least 2-fold to 20-fold. In some embodiments, the retained urine sample has a concentration that is increased by at least 2-fold, at least 3-fold, at least 4-fold, at least 5-fold, at least 10-fold, at least 15-fold, at least 20-fold, or more compared to the purified urine sample. In some embodiments, the retained urine sample has a concentration that is increased by at least 2-fold, at least 5-fold, at least 10-fold, or at least 15-fold compared to the purified urine sample. In some embodiments, the retained urine sample has a concentration that is increased by at least at least 5-fold compared to the purified urine sample. In some embodiments, the retained urine sample has a concentration that is increased by at least 10-fold compared to the purified urine sample.

As a result of passing at least a portion of the urine sample through a filter, the retained urine sample (comprising that portion of the sample subjected to the filtering that did not pass through the filter into the filtrate) will have a smaller volume as compared to the starting volume of the sample subjected to the filtering. In some embodiment, the retained urine sample has a volume that is at least 10% to at least 90% lower compared to the volume of the treated urine sample. In some embodiment, the retained urine sample has a volume that is at least 10%, at least 20%, at least 30% at least 40% at least 50%, at least 60%, at least 80%, or at least 90% lower compared to the volume of the treated urine sample. In some embodiment, the retained urine sample has a volume that is at least 50% lower compared to the volume of the treated urine sample. In some embodiment, the retained urine sample has a volume that is at least 75% lower compared to the volume of the treated urine sample. For example, when a treated urine sample of 50 mL is concentrated down to a retained urine sample volume of 5 mL or less, the retained urine sample volume will have been reduced by 90% or more.

The retained urine sample can be used immediately to further process or assay the cell-free nucleic acids. For instance, the retained urine sample may be processed an hour or less after concentrating. Alternatively, the retained urine sample may be stored for later use. In some instances, the retained urine sample may be stored at 4° C., 22° C. or 37° C. for up to a week after urine sample collection. In some embodiments, the retained urine sample may be frozen to preserve the sample for later analysis. In some embodiments, the retained urine sample is stored at −20° C., −80° C., or lower. In some embodiments, frozen samples are stored in a frozen state for one to six months.

The steps of treating, separating and concentrating the urine sample may be performed at various times after collection of the urine sample. In some embodiments, the treating is completed within 10 to 180 minutes after collection of the urine sample. In some embodiments, the treating is completed within 180, 150, 120, 90, 60, 45, 30, 15, or 10 minutes after collection of the urine sample. In some embodiments, the treating is completed within 120, 60 or 30 minutes after collection of the urine sample. In some embodiments, the treating is completed within 60 minutes after collection of the urine sample. In some embodiments, the treating is completed within 30 minutes after collection of the urine sample. In some embodiments, treated samples are stored prior to proceeding with the separating and concentrating steps. For example, after treating, the further steps of separating and concentrating may be completed within 1 to 14 days after collection (e.g., within 2, 3, 4, 5, 6, 7, 8, 9, or 10 days). In some embodiments, the separating and concentrating are completed within 7 days after urine sample collection.

Nucleic acids can be isolated from the retained urine sample by any of a variety of nucleic acid isolation methods. Exemplary methods include, but are not limited to, extraction, solid-phase extraction, silica-based purification, magnetic particle-based purification, phenol-chloroform extraction, chromatography, anion-exchange chromatography (using anion-exchange surfaces), electrophoresis, filtration, precipitation, immunoprecipitation, hybridization capture with targeted bait oligonucleotides, or any combination thereof. For instance, isolation of target nucleic acids by hybridization to biotinylated probes using a streptavidin-coated surface (e.g., streptavidin-coated beads). Commercially available methods and kits are also available, non-limiting examples of which include the QIAamp® Circulating Nucleic Acid Kit (QIAGEN), the Chemagic Circulating NA Kit (Chemagen), the NucleoSpin Plasma XS Kit (Macherey-Nagel), the High Pure Viral Nucleic Acid Large Volume Kit (Roche).

In some embodiments, the isolated cell-free nucleic acids comprise cfDNA, and the method comprises treating the cfDNA molecules to differentiate methylated nucleotides and unmethylated nucleotides, thereby generating converted cfDNA molecules. In some embodiments, the treatment comprises deamination, such as treating the cfDNA molecules with a cytosine deaminase or with bisulfite. In some embodiments, the method comprises treating the cfDNA molecules with bisulfite to generate the converted cfDNA molecules.

The methods disclosed herein may further comprise amplification of cell-free nucleic acids isolated from the retained urine sample. Amplification may be non-specific (e.g., amplification using random primers) or target-directed (e.g., directed to particular target regions of interest). Any suitable method known in the art may be used for amplification. Examples of nucleic acid amplification reactions include, but are not limited to polymerase chain reaction (PCR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), RT-PCR, loop mediated amplification (LAMP), exponential amplification reaction (EXPAR), and improved multiple displacement amplification (IMDA). Primer-based amplification methods may use primers targeting any region of the genome. Alternatively, primers may be used to specifically amplify targets/biomarkers of interest, thereby enriching the sample for desired targets/biomarkers. For example, forward and reverse primers can be prepared for each genomic region of interest and used to amplify fragments that correspond to or are derived from the desired genomic region. Amplification may be thermal amplification (e.g., as in PCR), or isothermal amplification.

In some embodiments, isolated cell-free nucleic acids, or amplification products thereof, are captured by hybridization to bait oligonucleotides. In some embodiments, the bait oligonucleotides are at least 45 nucleotides in length (e.g., at least 60, 75, 80, 90, 100, 110, or 120 nucleotides in length). In some embodiments, the bait oligonucleotides are no more than 130, 140, 150, 200, 250, or 300 bases in length. In some embodiments, the bait oligonucleotides are 45 to 300, 60 to 200, or 75 to 150 nucleotides in length. In some embodiments, the bait oligonucleotides are at least 50 nucleotides in length. In some embodiments, the bait oligonucleotides are at least 60 nucleotides in length. In some embodiments, the bait oligonucleotides are at least 75 nucleotides in length. In some embodiments, the indicated length of the bait oligonucleotide is the length that is designed to be complementary to a portion of a target genomic sequence, or a converted DNA molecule thereof.

In some embodiments, the bait oligonucleotides target at least 500, 1000, 1500, 5000, 10000, 12500, 15000, 17000, 19000, or more target genomic regions. In some embodiments, the bait oligonucleotides target fewer than 25000, 20000, 17000, 15000, 12500, 10000, or fewer target genomic regions. In some embodiments, the bait oligonucleotides target 5000 to 30000, 10000 to 25000, 12500 to 20000, or 15000 to 20000 target genomic regions. In some embodiments, the bait oligonucleotides target 100 to 500, 500 to 1000, 1500 to 5000, 5000 to 10000 target genomic regions. In some embodiments, the bait oligonucleotides target at least 500 target genomic regions. In some embodiments, the bait oligonucleotides target at least 1000 target genomic regions. In some embodiments, the bait oligonucleotides target at least 10000 target genomic regions. In some embodiments, the bait oligonucleotides target at least 15000 target genomic regions. In some embodiments, the bait oligonucleotides target fewer than 20000 target genomic regions.

In some embodiments, the bait oligonucleotides are configured to hybridize to converted DNA molecules (e.g., converted cfDNA molecules) corresponding to, or derived from, one or more genomic regions. Accordingly, the bait oligonucleotides can have a sequence different from the targeted genomic region. For example, a DNA containing an unmethylated CpG site can be converted to include UpG instead of CpG by deamination (e.g., by treatment with a cytosine deaminase or bisulfite). As a result, a bait oligonucleotide to such a target may be configured to hybridize to a sequence including UpG instead of a naturally-existing unmethylated CpG. Accordingly, a complementary site in the bait oligonucleotide to the unmethylated site can comprise CpA instead of CpG, and some probes targeting a hypomethylated site where all methylation sites are unmethylated may have no guanine (G) bases. In some embodiments, at least 3%, 5%, 10%, 15%, or 20% of the probes comprise no CpG sequences. In some embodiments, at least 5% of the probes comprise no CpG sequences. In some embodiments, at least 10% of the probes comprise no CpG sequences.

In some embodiments, the bait oligonucleotides are used to detect the presence or absence of cancer generally and/or provide a cancer classification such as cancer type, stage of cancer such as I, II, III, or IV, or provide the tissue of origin (TOO) where the cancer is believed to originate. The bait oligonucleotides may target genomic regions differentially methylated between general cancerous (pan-cancer) samples and non-cancerous samples, or only in cancerous samples with a specific cancer type (e.g., urological cancer-specific targets). For example, in some embodiments, bait oligonucleotides are designed to include differentially methylated genomic regions based on converted (e.g., bisulfite) sequencing data generated from the cell-free nucleic acids and/or whole genomic DNA of a set of cancer and non-cancer individuals.

In some embodiments, each of the target genomic regions is differentially methylated in a cancer sample relative to a non-cancer sample. In some embodiments, differential methylation comprises at least 50% to at least 90% of CpG sites in the target genomic region being methylated or unmethylated. In some embodiments, differential methylation comprises at least 50%, at least 60%, at least 70%, at least 80%, or at least 90% of CpG sites in the target genomic region being methylated or unmethylated. In some embodiments, differential methylation comprises at least 70% of CpG sites in the target genomic region being methylated or unmethylated. In some embodiments, differential methylation comprises at least 80% of CpG sites in the target genomic region being methylated or unmethylated. In some embodiments, the cancer is an urological cancer. In some embodiments, the cancer is a bladder cancer, a prostate cancer, or a kidney cancer. In some embodiments, the target genomic regions are selected to identify presence of, and optionally distinguish between, two or more cancer types (e.g., at least 3, 4, 5, or more cancer types).

In some embodiments, the cell-free nucleic acids comprise DNA or RNA. In some embodiments, the cell-free nucleic acids is cell-free DNA (cfDNA). In some embodiments, the method comprises treating the cfDNA molecules to differentiate methylated nucleotides and an unmethylated nucleotides, thereby generating the converted cfDNA molecules. In some embodiments, the treatment comprises deamination, such as treating the cfDNA molecules with a cytosine deaminase or with bisulfite. In some embodiments, the method comprises treating the cfDNA molecules with bisulfite to generate the converted cfDNA molecules.

In some embodiments, the method comprises separating bait-bound cell-free nucleic acid molecules from unbound cell-free nucleic acid molecules. In some embodiments, each bait oligonucleotide is conjugated to a solid surface (e.g., a chip or a bead, such as a magnetic or paramagnetic bead) or to a non-nucleotide affinity moiety (e.g., a member of a binding pair). In some embodiments, such conjugation is used to facilitate separation of cfDNA molecules bound to bait oligonucleotides from unbound cfDNA molecules. In some embodiments, the affinity moiety is biotin.

In some embodiments, the genomic regions can be selected to have at least 3, 5, 7, 10, or more methylation sites. In some embodiments, each target genomic region comprises at least five methylation sites. In some embodiments, the selected number of methylation sites (e.g., at least 5 methylation sites) are methylation sites that are differentially methylated in at least one type of cancer to be assayed by the panel. The selected target genomic regions can be located in various positions in a genome, including but not limited to promoters, enhancers, exons, introns, intergenic regions, and other parts.

In some embodiments, the target genomic regions are selected from sequences of genes in Table 1. In some embodiments, the target genomic region comprises a target sequence of a gene selected from Table 1. The target sequences may comprise two or more target sequences from a single gene, and/or one or more target sequences from two or more genes. In some embodiments, the target genomic region(s) comprise one or more target sequences from at least 1, 2, 3, 4, 5, 10, 15, 20, 25, 50, 75, or more genes selected from Table 1. In some embodiments, the target genomic regions comprise one or more target sequences from at least 10 genes from Table 1. In some embodiments, the target genomic regions comprise one or more target sequences from at least 15 genes from Table 1. In some embodiments, the target genomic regions comprise one or more target sequences from at least 25 genes from Table 1. In some embodiments, the target genomic region comprises a target sequence of a gene selected from one or more of TWIST1, EOMES, HOXA9, POU4F2, and ZNF154. In some embodiments, the target genomic region comprises a target sequence of a gene selected from one or more of TWIST1, PXDN, RP11-25902.3, EVX2, and KNDC1. In some embodiments, the target genomic region comprises a target sequence of a TWIST1 gene. In some embodiments, the target genomic regions comprise one or more target sequences from at least 20% of the genes from Table 1. In some embodiments, the target genomic regions comprise one or more target sequences from at least 25%, 30%, 40%, 50%, 60%, 70%, 80%, 90%, 95%, or 100% of the genes from Table 1. In some embodiments, the target genomic regions comprise one or more target sequences from at least 50% of the genes from of Table 1. In some embodiments, the target genomic region comprises target sequences from all of the genes of Table 1. In some embodiments, a target sequence is at least 10, 25, 35, 45, 50, 75, 100, 200, 300, or more nucleotides in length. In some embodiments, the target sequence is at least 25, at least 35, or at least 45 nucleotides in length. In some embodiments, the target sequence is at least 45 nucleotides in length. In some embodiments, the target genomic regions are diagnostic of one or more of bladder cancer, kidney cancer, or prostate cancer.

TABLE 1

| | | | |
|---|---|---|---|
| TWIST1 | EPN1 | HOXB6 | CTD-2313N18.5 |
| PXDN | LBH | TRAPPC9 | AC006076.1 |
| RP11-259O2.3 | HIST1H4D | ZFPM1 | TSC22D2 |
| EVX2 | HIST1H2APS4 | RP11-21B21.4 | HGFAC |
| KNDC1 | FZD2 | PON1 | RP11-271K21.11 |
| SEMA6B | ADARB2 | PON3 | UNKL |
| RP11-434B12.1 | PRDM2 | DENND1A | NAPA-AS1 |
| CRTC1 | FOXD4L1 | TRIP13 | NAPA |
| CDT1 | IGF2BP3 | TTC1 | MFHAS1 |
| C2orf43 | CAV2 | PWWP2A | RANGAP1 |
| HNRNPUL2- | CYTH3 | ABL1 | HOXB2 |
| BSCL2 | | | |
| HNRNPUL2 | RP11-1085N6.6 | DPF3 | HOXB-AS1 |
| FANCC | AC022182.1 | KDM4B | SHARPIN |
| CSNK1G2 | RASSF2 | LMAN2 | NACC2 |
| CPT1A | KCNQ4 | NCOR2 | BNIP3 |
| RP11-712B9.2 | RFTN1 | SLC2A1 | WDR37 |
| PSMG4 | RP11-807E13.3 | ASS1 | MUC5B |
| SLC22A23 | CTD-2201E18.1 | DTYMK | RP11-532E4.2 |
| PTCHD3P1 | AC053503.11 | SOX2-OT | RP11-445F12.1 |
| SVIL | ASIC4 | PACSIN2 | FBXW7 |
| ATXN7L1 | HOXB3 | AC008752.3 | CALM3 |
| MAP7D1 | HOXB-AS3 | CASKIN1 | RP11-770E5.1 |
| CARS | HOXB5 | FAM86C1 | |

In some embodiments, the target genomic regions are selected from sequences of genes in Table 2. In some embodiments, the target genomic region comprises a target sequence of a gene selected from Table 2. The target sequences may comprise two or more target sequences from a single gene, and/or one or more target sequences from two or more genes. In some embodiments, the target genomic region(s) comprise one or more target sequences from at least 1, 2, 3, 4, 5, 10, 15, 20, or more genes selected from Table 2. In some embodiments, the target genomic regions comprise one or more target sequences from at least 5 genes from Table 2. In some embodiments, the target genomic regions comprise one or more target sequences from at least 10 genes from Table 2. In some embodiments, the target genomic regions comprise one or more target sequences from at least 15 genes from Table 2. In some embodiments, the target genomic region comprises a target sequence of a gene selected from one or more of TWIST1, PXDN, RP11-25902.3, EVX2, and KNDC1. In some embodiments, the target genomic region comprises a target sequence of a TWIST1 gene. In some embodiments, the target genomic region comprises target sequences from all of the genes of Table 2. In some embodiments, a target sequence is at least 10, 25, 35, 45, 50, 75, 100, 200, 300, or more nucleotides in length. In some embodiments, the target sequence is at least 25, at least 35, or at least 45 nucleotides in length. In some embodiments, the target sequence is at least 45 nucleotides in length. In some embodiments, the target genomic regions are diagnostic of bladder cancer.

TABLE 2

| | | | |
|---|---|---|---|
| TWIST1 | RP11-434B12.1 | FANCC | PTCHD3P1 |
| PXDN | CRTC1 | CSNK1G2 | SVIL |
| RP11-259O2.3 | CDT1 | CPT1A | ATXN7L1 |
| EVX2 | C2orf43 | RP11-712B9.2 | MAP7D1 |
| KNDC1 | HNRNPUL2-BSCL2 | PSMG4 | CARS |
| SEMA6B | HNRNPUL2 | SLC22A23 | EPN1 |

In some embodiments, the target genomic regions are selected from sequences of genes in Table 3. In some embodiments, the target genomic region comprises a target sequence of a gene selected from Table 3. The target sequences may comprise two or more target sequences from a single gene, and/or one or more target sequences from two or more genes. In some embodiments, the target genomic region(s) comprise one or more target sequences from at least 1, 2, 3, 4, 5, 10, or more genes selected from Table 3. In some embodiments, the target genomic regions comprise one or more target sequences from at least 5 genes from Table 3. In some embodiments, the target genomic regions comprise one or more target sequences from at least 10 genes from Table 3. In some embodiments, the target genomic regions comprise one or more target sequences from at least 15 genes from Table 3. In some embodiments, the target genomic region comprises a target sequence of a gene selected from one or more of TWIST1, SEMA6B, PXDN, KNDC1, and RP11-25902.3. In some embodiments, the target genomic region comprises a target sequence of a TWIST1 gene. In some embodiments, the target genomic region comprises target sequences from all of the genes of Table 3. In some embodiments, a target sequence is at least 10, 25, 35, 45, 50, 75, 100, 200, 300, or more nucleotides in length. In some embodiments, the target sequence is at least 25, at least 35, or at least 45 nucleotides in length. In some embodiments, the target sequence is at least 45 nucleotides in length. In some embodiments, the target genomic regions are diagnostic of bladder cancer.

TABLE 3

| | | | |
|---|---|---|---|
| TWIST1 | FBXL19 | PSMG4 | CPT1A |
| SEMA6B | PTCHD3P1 | SLC22A23 | MAP7D1 |
| PXDN | SVIL | RP11-712B9.2 | CARS |
| KNDC1 | CRTC1 | ATXN7L1 | EPN |
| RP11-259O2.3 | | | |

In some embodiments, the target genomic regions are selected from sequences of genes in Table 4. In some embodiments, the target genomic region comprises a target sequence of a gene selected from Table 4. The target sequences may comprise two or more target sequences from a single gene, and/or one or more target sequences from two or more genes. In some embodiments, the target genomic region(s) comprise one or more target sequences from at least 1, 2, 3, 4, 5, 10, 15, 20, 30, or more genes selected from Table 4. In some embodiments, the target genomic regions comprise one or more target sequences from at least 10 genes from Table 4. In some embodiments, the target genomic regions comprise one or more target sequences from at least 15 genes from Table 4. In some embodiments, the target genomic regions comprise one or more target sequences from at least 25 genes from Table 4. In some embodiments, the target genomic region comprises a target sequence of a gene selected from one or more of LBH, HIST1H4D, HIST1H2APS4, FZD2, and ADARB2. In some embodiments, the target genomic region comprises target sequences from all of the genes of Table 4. In some embodiments, a target sequence is at least 10, 25, 35, 45, 50, 75, 100, 200, 300, or more nucleotides in length. In some embodiments, the target sequence is at least 25, at least 35, or at least 45 nucleotides in length. In some embodiments, the target sequence is at least 45 nucleotides in length. In some embodiments, the target genomic regions are diagnostic of prostate cancer.

TABLE 4

| | | | |
|---|---|---|---|
| LBH | KCNQ4 | PON1 | DTYMK |
| HIST1H4D | RFTN1 | PON3 | SOX2-OT |
| HIST1H2APS4 | RP11-807E13.3 | DENND1A | PACSIN2 |
| FZD2 | CTD-2201E18.1 | TRIP13 | AC008752.3 |
| ADARB2 | AC053503.11 | TTC1 | CASKIN1 |
| PRDM2 | ASIC4 | PWWP2A | FAM86C1 |
| FOXD4L1 | HOXB3 | ABL1 | CTD-2313N18.5 |
| IGF2BP3 | HOXB-AS3 | DPF3 | AC006076.1 |
| CAV2 | HOXB5 | KDM4B | TSC22D2 |
| CYTH3 | HOXB6 | LMAN2 | HGFAC |
| RP11-1085N6.6 | TRAPPC9 | NCOR2 | RP11-271K21.11 |
| AC022182.1 | ZFPM1 | SLC2A1 | UNKL |
| RASSF2 | RP11-21B21.4 | ASS1 | |

In some embodiments, the target genomic regions are selected from sequences of genes in Table 5. In some embodiments, the target genomic region comprises a target sequence of a gene selected from Table 5. The target sequences may comprise two or more target sequences from a single gene, and/or one or more target sequences from two or more genes. In some embodiments, the target genomic region(s) comprise one or more target sequences from at least 1, 2, 3, 4, 5, 10, 15, or more genes selected from Table 5. In some embodiments, the target genomic regions comprise one or more target sequences from at least 5 genes from Table 5. In some embodiments, the target genomic regions comprise one or more target sequences from at least 10 genes from Table 5. In some embodiments, the target genomic regions comprise one or more target sequences from at least 15 genes from Table 5. In some embodiments, the target genomic region comprises a target sequence of a gene selected from one or more of NAPA-AS1, NAPA, MFHAS1, RANGAP1, and HOXB2. In some embodiments, the target genomic region comprises target sequences from all of the genes of Table 5. In some embodiments, a target sequence is at least 10, 25, 35, 45, 50, 75, 100, 200, 300, or more nucleotides in length. In some embodiments, the target sequence is at least 25, at least 35, or at least 45 nucleotides in length. In some embodiments, the target sequence is at least 45 nucleotides in length. In some embodiments, the target genomic regions are diagnostic of kidney cancer.

TABLE 5

| | | | |
|---|---|---|---|
| NAPA-AS1 | HOXB-AS1 | MUC5B | RP11-770E5.1 |
| NAPA | SHARPIN | RP11-532E4.2 | HOXB3 |
| MFHAS1 | NACC2 | RP11-445F12.1 | HOXB-AS3 |
| RANGAP1 | BNIP3 | FBXW7 | DTYMK |
| HOXB2 | WDR37 | CALM3 | |

In some embodiments, methods disclosed herein comprise sequence isolated cell-free nucleic acid molecules. A variety of methods for sequencing nucleic acids are available, non-limiting examples of which include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators. In some embodiments, sequencing the nucleic acid molecules comprises sequencing amplicons of the isolated cell-free nucleic acid molecules.

In some embodiments, the method further comprises diagnosing a cancer in the subject. In some embodiments, the method further comprises treating the cancer in the subject. In some embodiments, the cancer is bladder cancer, prostate cancer, or kidney cancer. The particular mode of treatment may depend on one or more of various factors, such as the particular type of cancer detected, the cancer TOO, location, and stage. Non-limiting examples of treatment include surgical resection, radiation therapy, chemotherapy, and/or immunotherapy.

### Cancer Assay Panel

In one aspect, the present disclosure provides a cancer assay panel comprising a plurality of probes or a plurality of probe pairs. The assay panels described herein can alternatively be referred to as bait sets or as compositions comprising bait oligonucleotides. The probes can be polynucleotide-containing probes that are specifically designed to target (e.g., by way of complementarity) one or more genomic regions differentially methylated between cancer and non-cancer samples, between different cancer tissue of origin (TOO) types, between different cancer cell types, and/or between samples of different stages of cancer, as identified by methods provided herein. In some embodiments, the target genomic regions are selected to maximize classification accuracy, subject to a size budget (which is determined by sequencing budget and desired depth of sequencing).

In one aspect, the present disclosure provides a composition comprising a plurality of different bait oligonucleotides. In some embodiments, (a) the bait oligonucleotides hybridize to converted DNA molecules derived from one or more target genomic regions; (b) the one or more target genomic regions comprise one or more target sequences of one or more genes selected from Table 1; (c) the one or more target genomic regions are differentially methylated in a cancer; and (d) each bait oligonucleotide comprises a sequence at least 25 nucleotides in length that hybridizes to one of the target sequences. For designing the cancer assay panel, an analytics system may collect samples corresponding to various outcomes under consideration, e.g., samples from subjects known to have cancer, samples from subjects considered to be healthy, samples from a subject having a cancer with a known tissue of origin, etc. The sources of the DNA molecules (e.g., cfDNA and/or ctDNA) used to select target genomic regions can vary depending on the purpose of the assay. For example, different sources may be desirable for an assay intended to detect cancer generally, a specific type of cancer, a cancer stage, or a tissue of origin. These samples may be processed by whole-genome bisulfite sequencing (WGBS) or obtained from a public database (e.g., TCGA). In some embodiments, the analytics system is a computing system with a computer processor and a computer-readable storage medium with instructions for executing the computer processor to perform any or all operations described in this present disclosure. In some embodiments, at least some of the samples subjected to the analysis to identify target genomic regions are urine samples, such as urine samples process according to methods described herein.

The analytics system may then select target genomic regions based on methylation patterns of nucleic acid fragments. One approach considers pairwise distinguishability between pairs of outcomes for regions (or more specifically for CpG sites within regions). Another approach considers distinguishability for regions (or more specifically for CpG sites within regions) when considering each outcome against the remaining outcomes. From the selected target genomic regions with high distinguishability power, the analytics system may design probes to target fragments from the selected genomic regions. The analytics system may generate variable sizes of the cancer assay panel, e.g., where a small sized cancer assay panel includes probes targeting the most informative genomic regions, a medium sized cancer assay panel includes probes from the small sized cancer assay panel and additional probes targeting a second tier of informative genomic regions, and a large sized cancer assay panel includes probes from the small-sized and the medium-sized cancer assay panels along with even more probes targeting a third tier of informative genomic regions. With data obtained from such cancer assay panels (e.g., the methylation status on nucleic acids derived from the cancer assay panels), the analytics system may train classifiers with various classification techniques to predict a sample's likelihood of having a particular outcome or state, e.g., cancer, specific cancer type, other disorder, other disease, etc.

In an illustrative example, to design a cancer assay panel, an analytics system may collect information on the methylation status of CpG sites of nucleic acid fragments from samples corresponding to various outcomes under consideration, e.g., samples known to have cancer, samples considered to be healthy, samples from a known TOO, etc. These samples may be processed (e.g., with whole-genome bisulfite sequencing (WGBS)) to determine the methylation status of CpG sites, or the information may be obtained from TCGA. In some embodiments, the analytics system is a computing system with a computer processor and a computer-readable storage medium with instructions for executing the computer processor to perform any or all operations described in this present disclosure.

The analytics system may then select target genomic regions based on methylation patterns of nucleic acid fragments. One approach considers pairwise distinguishability between pairs of outcomes for regions (or more specifically CpG sites). Another approach considers distinguishability for regions (or more specifically CpG sites) when considering each outcome against the remaining outcomes. From the selected target genomic regions with high distinguishability power, the analytics system may design probes to target fragments from the selected genomic regions. The analytics system may generate variable sizes of the cancer assay panel, e.g., where a small sized cancer assay panel includes probes targeting the most informative genomic regions, a medium sized cancer assay panel includes probes from the small sized cancer assay panel and additional probes targeting a second tier of informative genomic regions, and a large sized cancer assay panel includes probes from the small-sized and the medium-sized cancer assay panels along with even more probes targeting a third tier of informative genomic regions. With such cancer assay panels, the analytics system may train classifiers with various classification techniques to predict a sample's likelihood of having a particular outcome or state, e.g., cancer, specific cancer type, other disorder, other disease, etc.

In some embodiments, the cancer assay panel comprises a plurality of pairs of probes, wherein each pair of the plurality of pairs comprises two probes configured to overlap each other by an overlapping sequence, wherein the overlapping sequence comprises at least 30 nucleotides, and wherein each probe is configured to hybridize to the same strand of an (optionally converted) DNA molecule (e.g., a cfDNA molecule) corresponding to one or more genomic regions. In some embodiments, each probe of the two probes in each pair of probes comprise a non-overlapping sequence of more than 20, 25, 30, 35, 40, 45, or 50 nucleotides relative to a different probe in the pair of probes. Thus, in some embodiments, a first probe and a second probe in a pair of probes can overlap by 30 nucleotides, wherein the first probe further comprises more than 20, 25, 30, 35, 40, 45, or 50 nucleotides that do not overlap with the second probe, and wherein the second probe further comprises more than 20, 25, 30, 35, 40, 45, or 50 nucleotides that do not overlap with the first probe. In some embodiments, each of the genomic regions comprises at least five methylation sites, and wherein the at least five methylation sites have an abnormal methylation pattern in cancerous samples or a different methylation pattern between samples of a different TOO. For example, in one embodiment, the at least five methylation sites are differentially methylated either between cancerous and non-cancerous samples or between one or more pairs of samples from cancers with different tissue of origin. In some embodiments, each pair of probes comprises a first probe and a second probe, wherein the second probe differs from the first probe. The second probe can overlap with the first probe by an overlapping sequence that is at least 30, at least 40, at least 50, or at least 60 nucleotides in length.

In some embodiments, each of the plurality of different bait oligonucleotides of a panel is at least 45 nucleotides in length (e.g., at least 60, 75, 80, 90, 100, 110, or 120 nucleotides in length). In some embodiments, each bait oligonucleotide in the plurality of probes is no more than 130, 140, 150, 200, 250, or 300 bases in length. In some embodiments, each bait oligonucleotide is 45 to 300, 60 to 200, or 75 to 150 nucleotides in length. In some embodiments, the bait oligonucleotides are at least 50 nucleotides in length. In some embodiments, the bait oligonucleotides are at least 60 nucleotides in length. In some embodiments, the bait oligonucleotides are at least 75 nucleotides in length. In some embodiments, the indicate length of the bait oligonucleotide is the length that is designed to be complementary to a portion of a target genomic sequence, or a converted DNA molecule thereof.

In some embodiments, the cancer assay panel is designed to target at least 5000, 10000, 12500, 15000, 17000, 19000, or more target genomic regions. In some embodiments, the cancer assay panel is designed to target fewer than 25000, 20000, 17000, 15000, 12500, 10000, or fewer target genomic regions. In some embodiments, the cancer assay panel is designed to target 5000 to 30000, 10000 to 25000, 12500 to 20000, or 15000 to 20000 target genomic regions. In some embodiments, the cancer assay panel is designed to target at least 10000 target genomic regions. In some embodiments, the cancer assay panel is designed to target at least 15000 target genomic regions. In some embodiments, the cancer assay panel is designed to target fewer than 20000 target genomic regions.

In some embodiments, the bait oligonucleotides are configured to hybridize to converted DNA molecules (e.g., converted cfDNA molecules) corresponding to, or derived from, one or more genomic regions. Accordingly, the bait oligonucleotides can have a sequence different from the targeted genomic region. For example, a DNA containing an unmethylated CpG site can be converted to include UpG instead of CpG by deamination (e.g., by treatment with a cytosine deaminase or bisulfite). As a result, a probe to such a target may be configured to hybridize to a sequence including UpG instead of a naturally-existing unmethylated CpG. Accordingly, a complementary site in the probe to the unmethylated site can comprise CpA instead of CpG, and some probes targeting a hypomethylated site where all methylation sites are unmethylated may have no guanine (G) bases. In some embodiments, at least 3%, 5%, 10%, 15%, or 20% of the probes comprise no CpG sequences. In some embodiments, at least 5% of the probes comprise no CpG sequences. In some embodiments, at least 10% of the probes comprise no CpG sequences.

In some embodiments, the cancer assay panel is used to detect the presence or absence of cancer generally and/or provide a cancer classification such as cancer type, stage of cancer such as I, II, III, or IV, or provide the TOO where the cancer is believed to originate. The panel may include probes targeting genomic regions differentially methylated between general cancerous (pan-cancer) samples and non-cancerous samples, or only in cancerous samples with a specific cancer type (e.g., bladder cancer-specific targets). For example, in some embodiments, a cancer assay panel is designed to include differentially methylated genomic regions based on converted (e.g., bisulfite) sequencing data generated from the cfDNA and/or whole genomic DNA of a set of cancer and non-cancer individuals.

In some embodiments, each of the target genomic regions is differentially methylated in at least one of a plurality of cancer types. In some embodiments, the plurality of cancer types comprises at least 2 cancer types (e.g., at least 2, 3, 3, 4, or more cancer types). In some embodiments, the plurality of cancer types comprises at least 2 cancer types. In some embodiments, the plurality of cancer types comprises at least 3 cancer types. In some embodiments, the plurality of cancer types include an urological cancer. In some embodiments, the plurality of cancer types include one or more of bladder cancer, urothelial cancer, kidney cancer, or prostate cancer.

Each of the probes (or probe pairs) may be designed to target one or more target genomic regions. The target genomic regions can be selected based on several criteria designed to increase selective enriching of informative cfDNA fragments (e.g., cfDNA fragments from a urine sample) while decreasing noise and non-specific bindings. Various filtering procedures for determining whether to include a target genomic regions are described herein. In some embodiments, two or more of the filtering procedures described herein are used in combination.

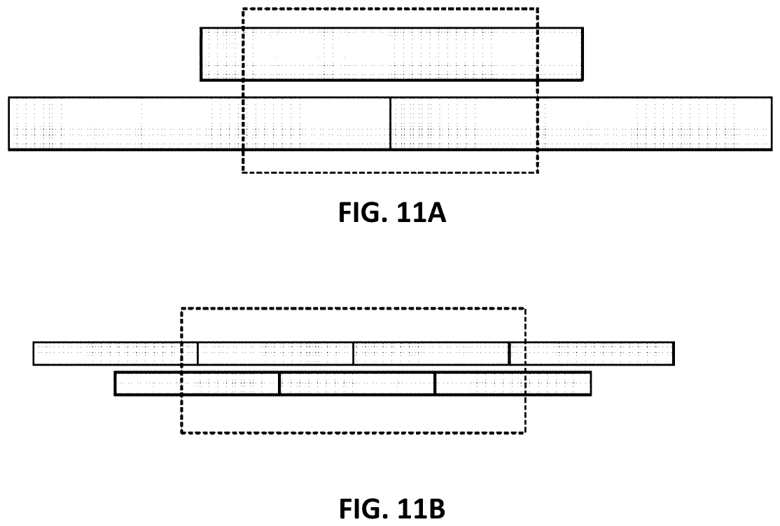

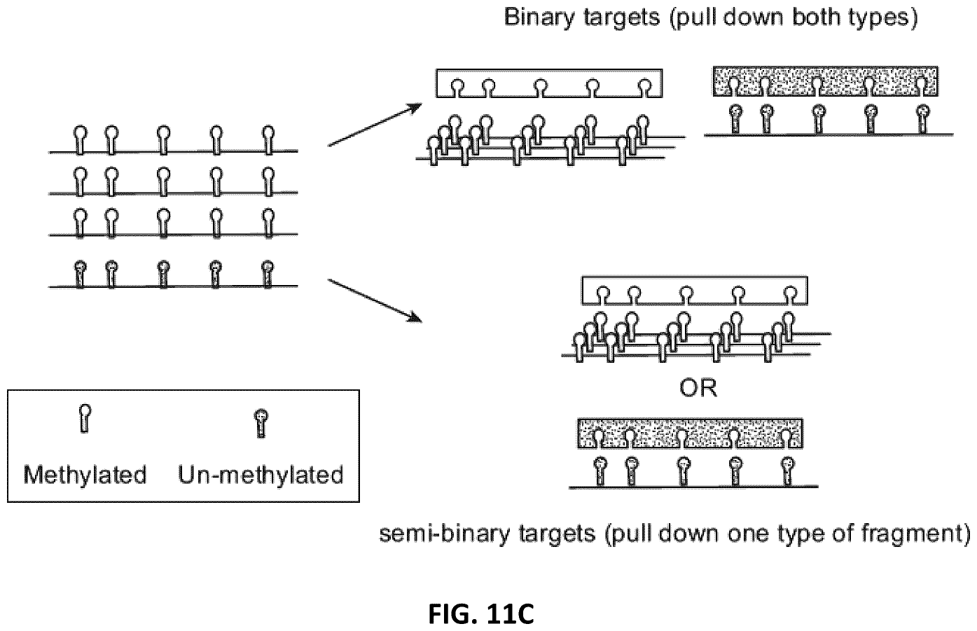

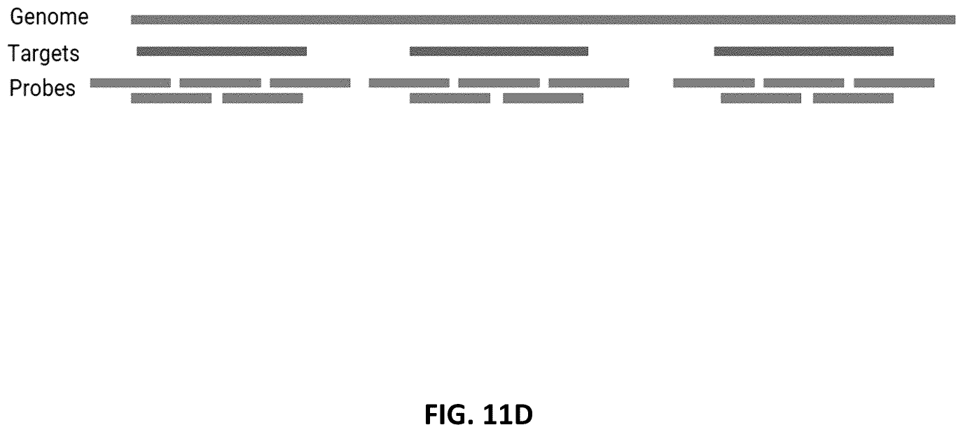

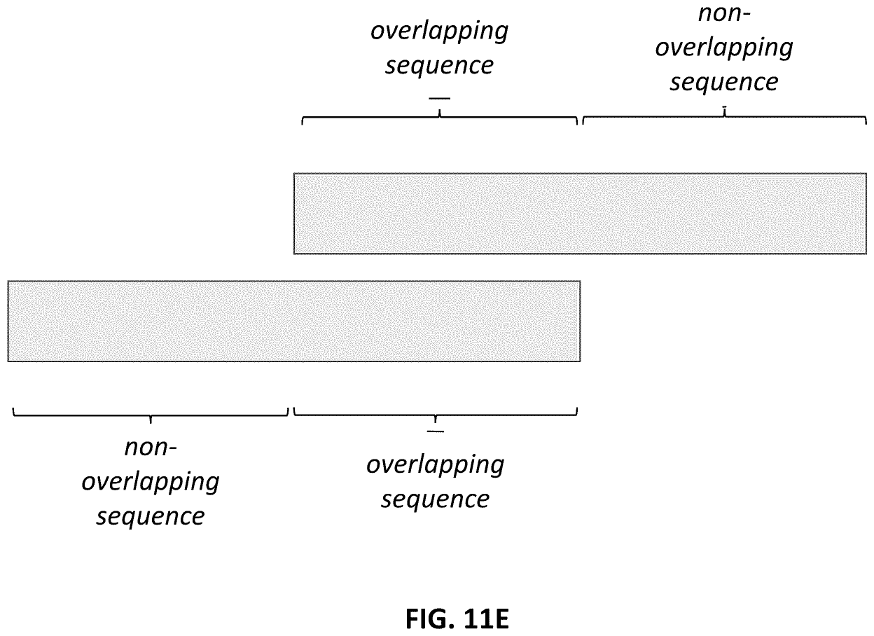

In one example, a panel can include probes that can selectively bind to and enrich cfDNA fragments that are differentially methylated in cancerous samples. In this case, sequencing of the enriched fragments can provide information relevant to detection of cancer. Furthermore, in some embodiments, the probes (or a portion thereof) are designed to target genomic regions that are determined to have an abnormal or anomalous methylation pattern in cancer samples, or in samples from certain cancer types, tissue types or cell types. In one embodiment, probes are designed to target genomic regions determined to be hypermethylated or hypomethylated in certain cancers or cancer types to provide additional selectivity and specificity of the detection. In some embodiments, a panel comprises probes targeting hypomethylated fragments. In some embodiments, a panel comprises probes targeting hypermethylated fragments. In some embodiments, a panel comprises both a first set of probes targeting hypermethylated fragments and a second set of probes targeting hypomethylated fragments. In some embodiments, a cancer assay panel includes not only probes that are designed to target a region that has a first methylation status (e.g., hypomethylation), but also includes probes that are designed to hybridize to the same target region with the opposite methylation status (e.g., hypermethylation). The targeting of probes to both hypo- and hypermethylated fragments from the same regions can be referred to as “binary” targeting (see, e.g., FIG. 11C). In some embodiments, the ratio between the first set of probes targeting hypermethylated fragments and the second set of probes targeting hypomethylated fragments (Hyper:Hypo ratio) ranges between 0.4 and 2, between 0.5 and 1.5, between 0.5 and 1.0, between 0.8 and 1, between 0.6 and 0.8 or between 0.4 and 0.6. In some embodiments, the Hyper:Hypo ratio is at least 2, 3, 4, 5, 6, 7, 8, 9, 10, or higher. In some embodiments, the Hyper:Hypo ratio is at least 5. Methods of identifying genomic regions (e.g., genomic regions giving rise to differentially methylated DNA molecules, or anomalously methylated DNA molecules) between cancer and non-cancer samples, between different cancer tissue of origin (TOO) types, between different cancer cell type, or between samples from different stages of cancer are provided in detail herein and methods of identifying anomalously methylated DNA molecules or fragments that are identified as indicative of cancer are also provided in detail herein.

In a second example, genomic regions can be selected when the genomic regions give rise to anomalously methylated DNA molecules in cancer samples or samples with known cancer tissue of origin (TOO) types. For example, as described herein, a Markov model trained on a set of non-cancerous samples can be used to identify genomic regions that give rise to anomalously methylated DNA molecules (e.g., DNA molecules having a methylation pattern below a p-value threshold).

In some embodiments, each of the probes targets a genomic region comprising at least 30 bp, 35 bp, 40 bp, 45 bp, 50 bp, 60 bp, 70 bp, 80 bp, 90 bp, 100 bp, 110 bp, 120 bp or more. In some embodiments, each of the probes targets a genomic region comprising 120 bp. In some embodiments, the genomic regions can be selected to have fewer than 30, 25, 20, 15, 12, 10, 8, or 6 methylation sites. In some embodiments, the genomic regions can be selected to have at least 3, 5, or 7 methylation sites. In some embodiments, each target genomic regions comprises at least five methylation sites. In some embodiments, the selected number of methylation sites (e.g., at least 5 methylation sites) are methylation sites that are differentially methylated in at least one type of cancer to be assayed by the panel.

In some embodiments, the genomic regions are selected as targets when at least 80, 85, 90, 92, 95, or 98% of the at least five methylation (e.g., CpG) sites within the region are either methylated or unmethylated in non-cancerous or cancerous samples, or in cancer samples from a tissue of origin (TOO).

In some embodiments, target genomic regions are filtered to select only those that are likely to be informative based on their methylation patterns, for example, CpG sites that are differentially methylated between cancerous and non-cancerous samples (e.g., abnormally methylated or unmethylated in cancer versus non-cancer), between cancerous samples of a TOO and cancerous samples of a different TOO, CpG sites that are differentially methylated only in cancerous samples of a TOO. For the selection, calculation can be performed with respect to each CpG or a plurality of CpG sites. For example, a first count can be determined that is the number of cancer-containing samples (cancer_count) that include a fragment overlapping that CpG, and a second count is determined that is the number of total samples containing fragments overlapping that CpG site (total). Genomic regions can be selected based on criteria positively correlated to the number of cancer-containing samples (cancer_count) that include a fragment indicative of cancer overlapping that CpG site, and inversely correlated with the number of total samples containing fragments indicative of cancer overlapping that CpG site (total). In one embodiment, the number of non-cancerous samples (n_{non-cancer}) and the number of cancerous samples (n_{cancer}) having a fragment overlapping a CpG site are counted. Then the probability that a sample is cancer is estimated, for example as (n_{cancer}+1)/(n_{cancer}+n_{non-cancer}+2). This principle could be similarly applied to other outcomes.

CpG sites scored by this metric can be ranked and greedily added to a panel until the panel size budget is exhausted. The process of selecting genomic regions indicative of cancer is further detailed herein. In some embodiments, different target regions may be selected depending on whether the assay is intended to be a pan-cancer assay or a single-cancer assay, or depending on what kind of flexibility is desired when picking which CpG sites are contributing to the panel. A panel for detecting a specific cancer type can be designed using a similar process. In this embodiment, for each cancer type, and for each CpG site, the information gain is computed to determine whether to include a probe targeting that CpG site. The information gain may be computed for samples with a given cancer type of a TOO compared to all other samples. For example, consider two random variables, “AF” and “CT”. “AF” is a binary variable that indicates whether there is an abnormal fragment overlapping a particular CpG site in a particular sample (yes or no). “CT” is a binary random variable indicating whether the cancer is of a particular type (e.g., bladder cancer or cancer other than bladder). One can compute the mutual information with respect to “CT” given “AF.” That is, how many bits of information about the cancer type (bladder vs. non-bladder in the example) are gained if one knows whether there is an anomalous fragment overlapping a particular CpG site. This can be used to rank CpG's based on how bladder-specific they are. This procedure is repeated for a plurality of cancer types. If a particular region is commonly differentially methylated only in bladder cancer (and not other cancer types or non-cancer), CpG's in that region would tend to have high information gains for bladder cancer. For each cancer type, CpG sites are ranked by this information gain metric, and then greedily added to a panel until the size budget for that cancer type is exhausted. In some embodiments, information gain is also measured against likelihood of pulling down a molecule comprising the target sequence, and/or the cost of sequencing that target. In some embodiments, a target region is only included if the ratio of information gain to cost of sequencing that target is above a threshold.

In some embodiments, filtration is performed to select probes with high specificity for enrichment (i.e., high binding efficiency) of nucleic acids derived from targeted genomic regions. Probes can be filtered to reduce non-specific binding (or off-target binding) to nucleic acids derived from non-targeted genomic regions. For example, probes can be filtered to select only those probes having fewer than a set threshold of off-target binding events (such as may be projected by a number of potential binding sites across the genome with a specified degree of complementarity over a given alignment window). In one embodiment, probes can be aligned to a reference genome (e.g., a human reference genome) to select probes that align to fewer than a set threshold of regions across the genome. In some embodiments, the reference genome is the human reference genome GRCh37/hg19, the sequence of which is available from Genome Reference Consortium and from the Genome Browser provided by Santa Cruz Genomics Institute. For example, probes can be selected that align to fewer than 25, 24, 23, 22, 21, 20, 19, 18, 17, 16, 15, 14, 13, 12, 11, 10, 9 or 8 off-target regions across the reference genome. In other cases, filtration is performed to remove genomic regions when the sequence of the target genomic regions appears more than 5, 10, 15, 20, 21, 22, 23, 24, 25, 26, 27, 28, 29, 30, 31, 32, 33, 34 or 35 times in a genome. Further filtration can be performed to select target genomic regions when a probe sequence, or a set of probe sequences that are 90%, 91%, 92%, 93%, 94%, 95%, 96%, 97%, 98% or 99% complementary to the target genomic regions, appear fewer than 25, 24, 23, 22, 21, 20, 19, 18, 17, 16, 15, 14, 13, 12, 11, 10, 9 or 8 times in a reference genome, or to remove target genomic regions when the probe sequence, or a set of probe sequences designed to enrich for the targeted genomic region are 90%, 91%, 92%, 93%, 94%, 95%, 96%, 97%, 98% or 99% complementary to the target genomic regions, appear more than 5, 10, 15, 20, 21, 22, 23, 24, 25, 26, 27, 28, 29, 30, 31, 32, 33, 34 or 35 times in a reference genome. This is for excluding repetitive probes that can pull down off-target fragments that are not desired and can impact assay efficiency.

In some experiments, a fragment-probe overlap of at least 45 bp was demonstrated to be effective for achieving a non-negligible amount of pulldown (though this number can very). Under some conditions, more than a 10% mismatch rate between the probe and fragment sequences in the region of overlap is sufficient to greatly disrupt binding, and thus pulldown efficiency. Therefore, sequences that can align to the probe along at least 45 bp with at least a 90% match rate can be candidates for off-target pulldown. Thus, in one embodiment, the number of such regions are scored. In some embodiments, the best probes have a score of 1, meaning they match in only one place (the intended target region). Probes with an intermediate score (say, less than 5 or 10) may in some instances be accepted, and in some instances any probes above a particular score are discarded. In some embodiments, a candidate probe is excluded from the assay panel if it contains a sequence of 45 contiguous bp with at least 90% complementarity to more than 20 off-target sites. Other cutoff values can be used for specific samples.

Once the probes hybridize and capture DNA fragments corresponding to, or derived from, a target genomic region, the hybridized probe-DNA fragment intermediates are pulled down (or isolated), and the targeted DNA is amplified and sequenced. The sequence read provides information relevant for detection of cancer. For this end, a panel can be designed to include a plurality of probes that can capture fragments that can together provide information relevant to detection of cancer. In some embodiments, a panel includes at least 10, 20, 30, 40, 50, 60, 80, 100, 150, 200, 300, 500, 1,000, 2,000, 2,500, 5,000, 6,000, 7,500, 10,000, 15,000, 20,000, 25,000, or 30,000 different pairs of probes. In some embodiments, a panel includes at least 20, 30, 40, 60, 80, 100, 120, 160, 200, 300, 400, 600, 1,000, 2,000, 5,000, 6,000, 10,000, 12,000, 15,000, 20,000, 30,000, 40,000, 50,000, or 60,000 different probes. The plurality of different probes together can comprise at least 2,000, 5,000, 10,000, 20,000, 40,000, 60,000, 80,000, 100,000, 0.2 million, 0.4 million, 0.6 million, 0.8 million, 1 million, 2 million, 3 million, 4 million, or 5 million nucleotides. In some embodiments, a panel includes no more than 20, 30, 40, 50, 60, 80, 100, 150, 200, 300, 500, 1,000, 2,000, 2,500, 5,000, 6,000, 7,500, 10,000, 15,000, 20,000, 25,000, or 30,000, or 40,000 different pairs of probes. In other embodiments, a panel includes no more than 30, 40, 60, 80, 100, 120, 160, 200, 300, 400, 600, 1,000, 2,000, 5,000, 6,000, 10,000, 12,000, 15,000, 20,000, 30,000, 40,000, 50,000, 60,000, or 70,000 different probes.

The selected target genomic regions can be located in various positions in a genome, including but not limited to promoters, enhancers, exons, introns, intergenic regions, and other parts. In some instances, primers may be used to specifically amplify targets/biomarkers of interest (e.g., by PCR), thereby enriching the sample for desired targets/biomarkers (optionally without hybridization capture). For example, forward and reverse primers can be prepared for each genomic region of interest and used to amplify fragments that correspond to or are derived from the desired genomic region. Thus, while the present disclosure pays particular attention to cancer assay panels and bait sets for hybridization capture, the disclosure is broad enough to encompass other methods for enrichment of cell-free nucleic acid molecules (e.g., cfDNA). Accordingly, a skilled artisan, with the benefit of this disclosure, will recognize that methods analogous to those described herein in connection with hybridization capture can alternatively be accomplished by replacing hybridization capture with some other enrichment strategy, such as PCR amplification of cell-free DNA fragments that correspond with genomic regions of interest. In some embodiments, bisulfite padlock probe capture is used to enrich regions of interest, such as is described in Zhang et al. (US 2016/0340740). In some embodiments, additional or alternative methods are used for enrichment (e.g., non-targeted enrichment) such as reduced representation bisulfite sequencing, methylation restriction enzyme sequencing, methylation DNA immunoprecipitation sequencing, methyl-CpG-binding domain protein sequencing, methyl DNA capture sequencing, or microdroplet PCR.

### Probes

The cancer assay panels (alternatively referred to as “bait sets”) provided herein can be a panel that includes a set of hybridization probes (also referred to herein as “probes”) designed to, during enrichment, target and pull-down nucleic acid fragments of interest for the assay. In some embodiments, the probes are designed to hybridize and enrich DNA or cfDNA molecules from cancerous samples that have been treated to convert unmethylated cytosines (C) to uracils (U). In other embodiments, the probes are designed to hybridize to and enrich DNA or cfDNA molecules from cancerous samples of a TOO (or a plurality of TOOs) that have been treated to convert unmethylated cytosines (C) to uracils (U). The probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. In a particular embodiment, a cancer assay panel may include sets of two probes, one probe targeting the positive strand and the other probe targeting the negative strand of a target genomic region.

For each target genomic region, at least four possible probe sequences can be designed. Each target region is double-stranded, and as such, a probe or probe set can target either the “positive” or forward strand or its reverse complement (the “negative” strand). Additionally, in some embodiments, the probes or probe sets are designed to enrich DNA molecules or fragments that have been treated to convert unmethylated cytosines (C) to uracils (U). For probes or probe sets that are designed to enrich DNA molecules corresponding to, or derived from the targeted regions after conversion, the probe's sequence can be designed to enrich DNA molecules of fragments where unmethylated C's have been converted to U's (by utilizing A's in place of G's at sites that are unmethylated cytosines in DNA molecules or fragments corresponding to, or derived from, the targeted region). In one embodiment, probes are designed to bind to, or hybridize to, DNA molecules or fragments from genomic regions known to contain cancer-specific methylation patterns (e.g., hypermethylated or hypomethylated DNA molecules), thereby enriching for cancer-specific DNA molecules or fragments. Targeting genomic regions, or cancer-specific methylation patterns, can be advantageous allowing one to specifically enrich for DNA molecules or fragments identified as informative for cancer or cancer TOO, and thus, lowering sequencing needs and sequencing costs. In other embodiments, two probe sequences can be designed per a target genomic region (one for each DNA strand). In still other cases, probes are designed to enrich for all DNA molecules or fragments corresponding to, or derived from, a targeted region (i.e., regardless of strand or methylation status). This might be because the cancer methylation status is not highly methylated or unmethylated, or because the probes are designed to target small mutations or other variations rather than methylation changes, with these other variations similarly indicative of the presence or absence of a cancer or the presence or absence of a cancer of one or more TOOs. In that case, all four possible probe sequences can be included per a target genomic region.

In some embodiments, probes range in length from 10 s, 100 s, 200 s, or 300 s of base pairs. The probes can comprise at least 50, 75, 100, or 120 nucleotides. The probes can comprise less than 300, 250, 200, or 150 nucleotides. In an embodiment, the probes comprise 100-150 nucleotides. In one particular embodiment, the probes comprise 120 nucleotides.

In some embodiments, the probes are designed in a “2×tiled” fashion to cover overlapping portions of a target region. Each probe optionally overlaps in coverage at least partially with another probe in the library. In such embodiments, the panel contains multiple pairs of probes, with each probe in a pair overlapping the other by at least 25, 30, 35, 40, 45, 50, 60, 70, 75 or 100 nucleotides. In some embodiments, the overlapping sequence can be designed to be complementary to a target genomic region (or cfDNA derived therefrom) or to be complementary to a sequence with homology to a target region or cfDNA. Thus, in some embodiments, at least two probes comprise a sequence with complementarity to the same sequence within a target genomic region, and a nucleotide fragment corresponding to or derived from the target genomic region can be bound and pulled down by at least one of the probes. For a given pair of probes comprising an overlapping sequence, the pair may comprise non-overlapping sequences with complementarity to the target genomic region extending from different ends of the overlapping sequence (see, e.g., FIG. 11E). Other levels of tiling are possible, such as 3× tiling, 4× tiling, etc., wherein each nucleotide in a target region can bind to more than two probes.

In one embodiment, a single base in a target genomic region is overlapped by exactly two probes, as illustrated in FIG. 11B. Probes that extend in both directions beyond a target genomic region are useful to pull down cfDNA fragments comprising a portion of the target genomic region and DNA sequences adjacent to the target genomic region. In some instances, even relatively small target regions may be targeted with three probes (see, e.g., FIG. 11A). A probe set comprising three or more probes is optionally used to capture a larger genomic region (see, e.g., FIG. 11B). In some embodiments, subsets of probes will collectively extend across an entire target genomic region (e.g., may be complementary to non-converted or converted fragments from the entire genomic region). A tiled probe set optionally comprises probes that collectively include at least two probes that overlap every nucleotide in the target genomic region. This is done to ensure that cfDNAs comprising a small portion of a target genomic region at one end will have a substantial overlap extending into the adjacent non-targeted genomic region with at least one probe, to provide for efficient capture.

In some embodiments, each target genomic region is targeted by a set of probes. Probe sets can be designed in a tiled fashion such that adjacent probes have overlapping sequences that hybridize to the same portion of a genomic region (see FIG. 11D). As DNA has two strands, a probe set may also include overlapping probes that hybridize to the other strand, for a total of four probes that hybridize to the same portion of a genomic region.

In some embodiments, a set of probes configured to hybridize to a target genomic region does not span the entire region—i.e. at least some sequences within the target genomic region do not have a corresponding probe. For example, a sequence within a target genomic region may be similar or identical to many other sequences in the genome and no probe is designed to target this sequence because such a probe would hybridize to a number of off-target regions above a threshold number.

For example, a 100 bp cfDNA fragment comprising a 30 nt target genomic region will have at least 65 bp overlap with at least one of the overlapping probes. Other levels of tiling are possible. For example, to increase target size and add more probes in a panel, probes can be designed to expand a 30 bp target region by at least 70 bp, 65 bp, 60 bp, 55 bp, or 50 bp. To capture any fragment that overlaps the target region at all (even if by only 1 bp), the probes can be designed to extend past the ends of the target region on either side, such as by at least 50 bp, 55 bp, 60 bp, 65 bp 70 bp, 75 bp, 80 bp, or 85 bp. The probes can be designed to extend past the ends of the target region on either side by 75 bp. In some embodiments, the existence of a probe designed to extend past an end of a target genomic region does not increase the size of the target genomic region (e.g., is not included in determining the size of the respective target genomic region or the collective size of a plurality of target genomic regions).

In some embodiments, each bait oligonucleotide is conjugated to a solid surface (e.g., a chip or a bead, such as a magnetic or paramagnetic bead) or to a non-nucleotide affinity moiety (e.g., a member of a binding pair). In some embodiments, such conjugation is used to facilitate separation of DNA molecules bound to bait oligonucleotides from unbound DNA molecules. In general, “binding pair” refers to one of a first and a second moiety, wherein the first and the second moiety have a specific binding affinity for each other. Non-limiting examples of binding pairs include antigens/antibodies; biotin/avidin (or biotin/streptavidin); calmodulin binding protein (CBP)/calmodulin; hormone/hormone receptor; lectin/carbohydrate; peptide/cell membrane receptor; enzyme/cofactor; and enzyme/substrate. In some embodiments, the affinity moiety is biotin.

In some embodiments, target genomic regions are selected such that application of a trained classifier to sequences of the converted DNA molecules captured by the bait oligonucleotides discriminates a subject with cancer from a subject without cancer with a defined specificity and/or sensitivity. Methods for the selection of target regions, classifier training, and selected specificities and sensitivities useful in the detection of various cancer types are disclosed herein, such as in connection with various aspects relating to methods herein. In some embodiments, the classifier is a binary classifier, a mixture model classifier, or a multilayer perceptron model classifier. In some embodiments, the defined specificity for each of the plurality of cancer types is 0.900 or higher (e.g., at least 0.950, 0.975, 0.980, 0.985, 0.990, 0.995, or higher). In some embodiments, the application of the trained classifier comprises a sensitivity of at least 30% (e.g., at least 40%, 50%, 60%, 70%, 80%, or higher) for each of the plurality of cancer types.

In some embodiments, the target genomic regions are selected from sequences of genes in Table 1. In some embodiments, the cancer assay panel comprises a plurality of probes, wherein each of the plurality of probes is configured to hybridize to a DNA molecule (e.g., a converted DNA molecule), or an amplicon thereof, corresponding to one or more genomic regions comprising a target sequence from one or more genes of Table 1. In some embodiments, the cancer assay panel comprises a plurality of probes, wherein each of the plurality of probes is configured to hybridize to a DNA molecule (e.g., a converted DNA molecule), or amplicon thereof, corresponding to at least 1, 2, 3, 4, 5, 10, 15, 20, 25 or more target sequences of one or more genes selected from Table 1. In some embodiments, the target genomic region comprises at least 10 target sequences of one or more genes from Table 1. In some embodiments, the target genomic region comprises at least 15 target sequences of one or more genes from Table 1. The target sequences may comprise two or more target sequences from a single gene, and/or one or more target sequences from two or more genes. In some embodiments, the target genomic region comprises a target sequence of a gene selected from TWIST1, EOMES, HOXA9, POU4F2, and ZNF154. In some embodiments, the target genomic region comprises a target sequence of a TWIST1 gene. In some embodiments, the target genomic region comprises target sequences from at least 20% of the genes of Table 1. In some embodiments, the target genomic region comprises target sequences from at least 25%, 30%, 40%, 50%, 60%, 70%, 80%, 90%, 95%, or 100% of the genes from Table 1. In some embodiments, the target genomic region comprises sequences from at least 50% of the genes from of Table 1. In some embodiments, the target genomic region comprises target sequences from all of the genes of Table 1. In some embodiments, a target sequence is at least 10, 25, 35, 45, 50, 75, 100, 200, 300, or more nucleotides in length. In some embodiments, the target sequence is at least 25, at least 35, or at least 45 nucleotides in length. In some embodiments, the target sequence is at least 45 nucleotides in length. In some embodiments, each target genomic region comprises at least five methylation sites. In some embodiments, the differential methylation comprises at least 80% of CpG sites in the target genomic region being methylated or unmethylated.

### Methods of Selecting Target Genomic Regions

In one aspect, the present disclosure provides methods of selecting target genomic regions for detecting cancer and/or a TOO. In some embodiments, the target genomic regions are abnormally or anomalously methylated in cancer and/or a TOO. The targeted genomic regions can be used to design and manufacture probes for a cancer assay panel. Methylation status of DNA or cfDNA molecules corresponding to, or derived from, the target genomic regions can be screened using the cancer assay panel. Alternative methods, for example by WGBS or other methods, can be also implemented to detect methylation status of DNA molecules or fragments corresponding to, or derived from, the target genomic regions.

### Sample Processing

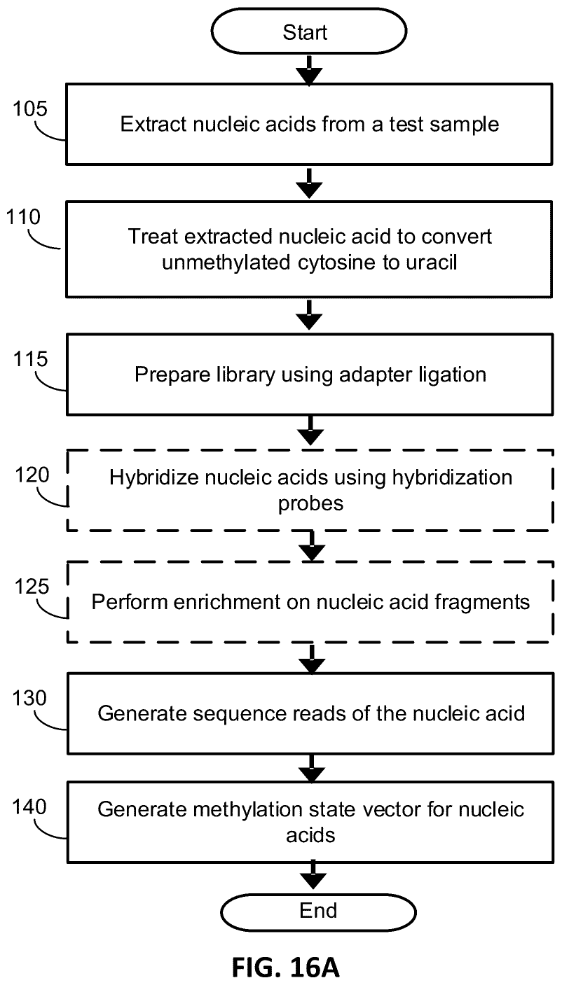

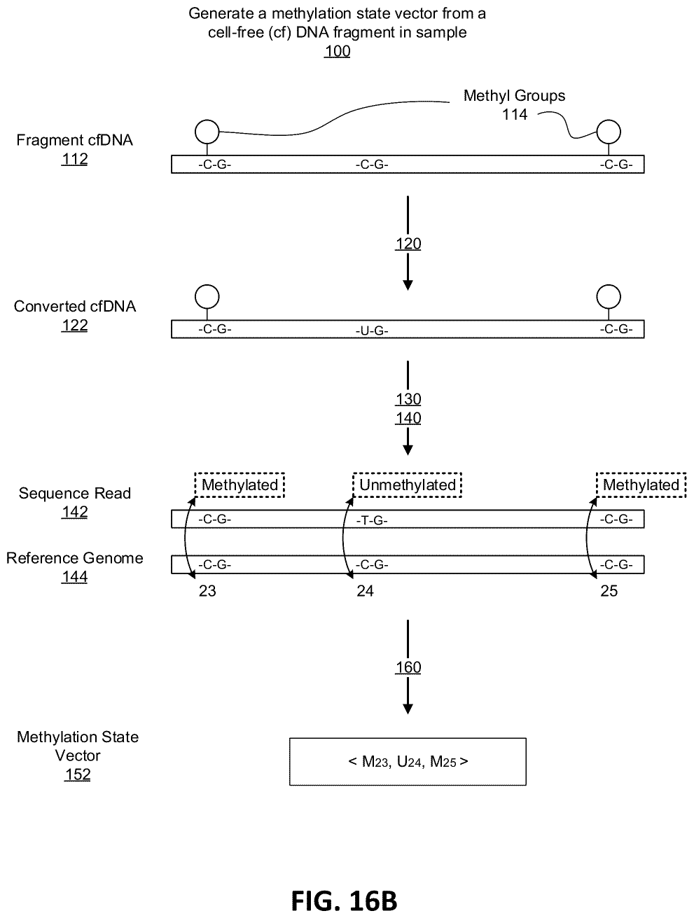

FIG. 16A is a flowchart of a process for processing a nucleic acid sample and generating methylation state vectors for DNA fragments, according to one embodiment. The method includes, but is not limited to, the following steps. For example, any step of the method may comprise a quantitation sub-step for quality control or other laboratory assay procedures known to one skilled in the art.

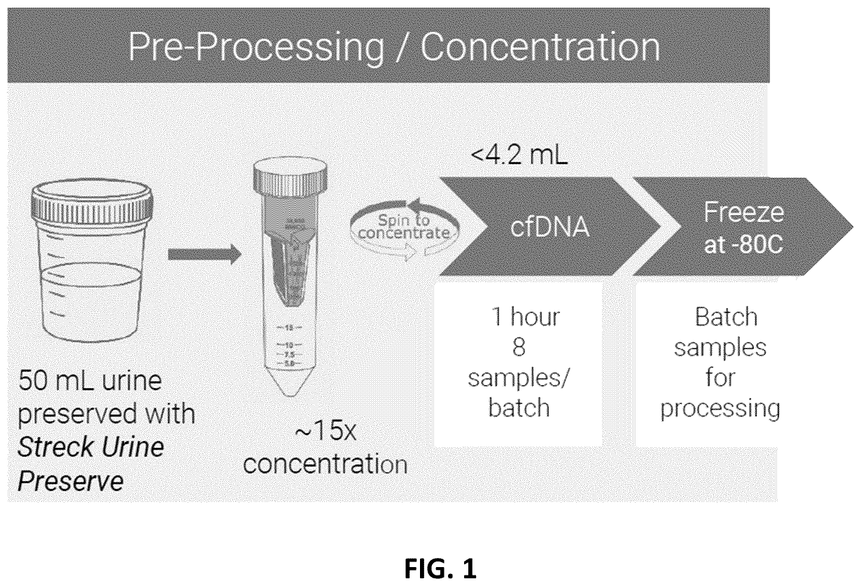

In step 105, a urine sample comprising nucleic acid (e.g., cfDNA) is collected from a subject. The sample may be any subset of the human genome, including the whole genome. The urine sample may be prepared by any of the methods for collecting nucleic acids from urine disclosed herein. FIG. 1 shows an exemplary method for urine sample processing. In this particular embodiment, urine is collected from a subject, treated with cell lysis inhibitor, and centrifuged to sediment and remove cellular debris. The urine sample is then concentrated by filtration, resulting in a concentrated urine sample having an increased conetration of nucleic acids and a lower volume relative to the untreated urine sample. The concentrated urine sample may then be subjected to nucleic acid extraction and library preparation, which can then be sequenced for methylation analysis and detection of a cancer (e.g., bladder, kidney or prostate cancer). The extracted sample may comprise cfDNA and/or ctDNA. For healthy individuals, the human body may naturally clear out cfDNA and other cellular debris. If a subject has a cancer or disease, cfDNA and/or ctDNA in a sample may be present at a detectable level for detecting the cancer or disease.

In step 110, the nucleic acids are treated to convert unmethylated cytosines to uracils. In one embodiment, the method uses a bisulfite treatment of DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

In step 115, a sequencing library is prepared. In some embodiments, a ssDNA adapter is added to the 3′-OH end of a bisulfite-converted ssDNA molecule using a ssDNA ligation reaction. In one embodiment, the ssDNA ligation reaction uses CircLigase II (Epicentre) to ligate the ssDNA adapter to the 3′-OH end of a bisulfite-converted ssDNA molecule, wherein the 5′-end of the adapter is phosphorylated and the bisulfite-converted ssDNA has been dephosphorylated (i.e., the 5′ phosphate is removed). In another embodiment, the ssDNA ligation reaction uses Thermostable 5′ AppDNA/RNA ligase (available from New England BioLabs (Ipswich, MA)) to ligate the ssDNA adapter to the 3′-OH end of a bisulfite-converted ssDNA molecule. In this example, a first adapter is adenylated at the 5′-end and blocked at the 3′-end. In another embodiment, the ssDNA ligation reaction uses a T4 RNA ligase (available from New England BioLabs) to ligate the ssDNA adapter to the 3′-OH end of a bisulfite-converted ssDNA molecule. In a second step, a second strand DNA is synthesized in an extension reaction. For example, an extension primer, that hybridizes to a primer sequence included in the ssDNA adapter, is used in a primer extension reaction to form a double-stranded bisulfite-converted DNA molecule. Optionally, in one embodiment, the extension reaction uses an enzyme that is able to read through uracil residues in the bisulfite-converted template strand. Optionally, in a third step, a dsDNA adapter is added to the double-stranded bisulfite-converted DNA molecule. Finally, the double-stranded bisulfite-converted DNA is amplified to add sequencing adapters. For example, PCR amplification using a forward primer that includes a P5 sequence and a reverse primer that includes a P7 sequence is used to add P5 and P7 sequences to the bisulfite-converted DNA. Optionally, during library preparation, unique molecular identifiers (UMI) may be added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs comprise degenerate base pair positions that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis (either by the UMI alone, or using the UMI in combination with a portion of the sample nucleic acid fragment end sequence, such as the first 2-10 nucleotides).

In step 120, targeted DNA sequences may be enriched from the library. This is used, for example, where a targeted panel assay is being performed on the samples. During enrichment, hybridization probes (also referred to herein as “probes” or “bait oligonucleotides”) are used to target, and optionally pull down, nucleic acid fragments informative for the presence or absence of cancer (or disease), cancer status, or a cancer classification (e.g., cancer type or tissue of origin). In some embodiments, the probes have features specified herein, such as in connection with various other aspects described herein. For a given workflow, the probes may be designed to anneal (or hybridize) to a target (complementary) strand of DNA (e.g., converted DNA molecules). The target strand may be the “positive” strand (e.g., the strand transcribed into mRNA, and subsequently translated into a protein) or the complementary “negative” strand. The probes may range in length from 10s, 100s, or 1000s of base pairs. Moreover, the probes may cover overlapping portions of a target region, as described herein.

After a hybridization step 120, the hybridized nucleic acid fragments are enriched (e.g., by capturing or otherwise separating from unbound nucleic acids) and may also be amplified using PCR (enrichment 125). For example, the target sequences can be enriched to obtain enriched sequences that can be subsequently sequenced. In general, a variety of methods can be used to isolate, and enrich for, probe-hybridized target nucleic acids. For example, a biotin moiety can be added to the 5′-end of the probes (i.e., biotinylated) to facilitate isolation of target nucleic acids hybridized to probes using a streptavidin-coated surface (e.g., streptavidin-coated beads).

In step 130, sequence reads are generated from the enriched nucleic acid fragments. Sequencing data may be acquired from the enriched DNA sequences by known means in the art. For example, the method may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In step 140, methylation state vectors are generated from the sequence reads. To do so, a sequence read is aligned to a reference genome. The reference genome helps provide the context as to what position in a human genome the DNA fragment (e.g., cfDNA) originates from. In a simplified example, the sequence read is aligned such that the three CpG sites correlate to CpG sites 23, 24, and 25 (arbitrary reference identifiers used for convenience of description). After alignment, there is information both on methylation status of all CpG sites on the cfDNA fragment and which position in the human genome the CpG sites map to. With the methylation status and location, a methylation state vector may be generated for the DNA fragment.

Alternative methods for detection of methylation patterns can include the use of oligonucleotide microarrays and selective PCR amplification. A method of detection of methylation patterns can comprises use of an oligonucleotide microarray. The method can comprise labeling cfDNA fragments with a label. The cfDNA fragments can be from an individual. The cfDNA fragments can be bisulfite-converted ssDNA molecules or converted cfDNA fragments. The label can be a fluorescent label. The fluorescent label can be a cyanine dye. The cyanine dye can be Cy3 or Cy5, DY547, or DY647. An oligonucleotide microarray for use in the detection can comprise at least 75, 150, 300, or 1000 pairs of bait oligonucleotides. In some embodiments, each pair of the bait oligonucleotides comprise a first bait and a second bait. The first bait can comprise an overlapping sequence and a first non-overlapping sequence. The second bait can comprise the overlapping sequence and a second non-overlapping sequence. The overlapping sequence can comprise at least 30, 40, 50, or 60 nucleotides. The first non-overlapping sequence and the second non-overlapping sequence can comprise more than 30, 40, 50, or 60 nucleotides. The first bait and the second bait of each pair of baits oligonucleotides can be configured to hybridize to a converted cfDNA fragment derived from a genomic region comprising at least five methylation sites that are differentially methylated in cfDNA fragments from individuals with cancer compared to cfDNA fragments from individuals without cancer. The converted cfDNA fragment can be labeled with a first fluorescent label. In some embodiments, reference cfDNA fragments are labeled with a second fluorescent label. Reference cfDNA fragments can be cfDNA fragments with a known methylation pattern. The second fluorescent label can be a cyanine dye. The cyanine dye can be Cy3 or Cy5, DY547, or DY647. The first fluorescent label and the second fluorescent label can be different. In some embodiments, a methylation pattern is detected by contacting labeled cfDNA fragments from an individual and labeled reference cfDNA fragments to an oligonucleotide microarray. The methylation pattern can be determined by comparing amounts of the first fluorescent label and the second fluorescent label associated with each array position on the microarray. Each array position can correspond to a pair of bait oligonucleotides. In some embodiments, the methylation pattern is associated with a cancer or a cancer type. The method can comprise applying a trained classifier to predict a likelihood of cancer. In some embodiments, application of the trained classifier to sequences of the converted cfDNA molecules that hybridize to the bait oligonucleotides can discriminate a subject with the cancer or the cancer type from a subject without cancer with a specificity of 0.990 or 0.994 and a sensitivity of at least 40%, at least 45%, or at least 50%.

A method of detection of methylation patterns can comprises use of selective PCR amplification. A composition comprising a plurality of pairs of oligonucleotides configured to hybridize to converted DNA fragments derived from target genomic regions, wherein each pair of oligonucleotides is configured for selective PCR amplification of a sequence. In some embodiments, the sequence for selective PCR amplification comprises converted DNA derived from a hypermethylated target genomic region to yield a PCR product and cannot amplify converted cfDNA derived from a hypomethylated target genomic region. In some embodiments, the sequence for selective PCR amplification comprises converted DNA derived from a hypomethylated target genomic region to yield a PCR product and cannot amplify converted cfDNA derived from a hypermethylated target genomic region. Each target genomic region can comprise at least five CpG sites. In some embodiments, the target genomic region comprises at least 1, 2, 3, 4, 5, 10, 15 or more of the target genomic regions comprising a target sequence from one or more genes selected from Table 1. In some embodiments, the target genomic region comprises a target sequence of a gene selected from TWIST1, EOMES, HOXA9, POU4F2, and ZNF154. Determining a likelihood of a presence or absence of a cancer or cancer type in a subject can comprise amplifying converted cfDNA fragments from the subject with the pair of oligonucleotides is configured for selective PCR amplification described herein, sequencing captured DNA fragments, and applying a trained classifier to the DNA sequences to determining the likelihood. The sensitivity of the classifier for determining the likelihood of the cancer or the cancer type can be at least 40% with a specificity of 0.990 or higher.

### Generation of Data Structure

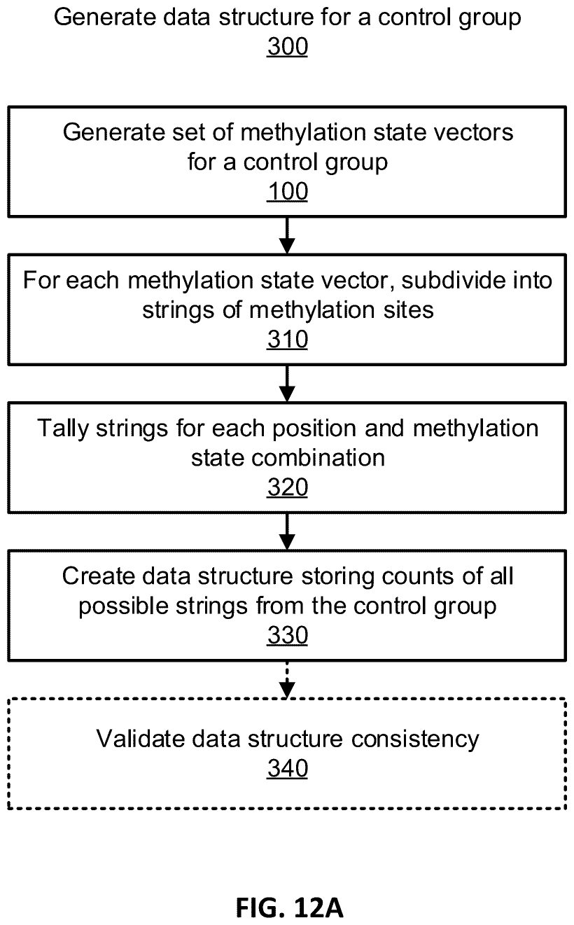

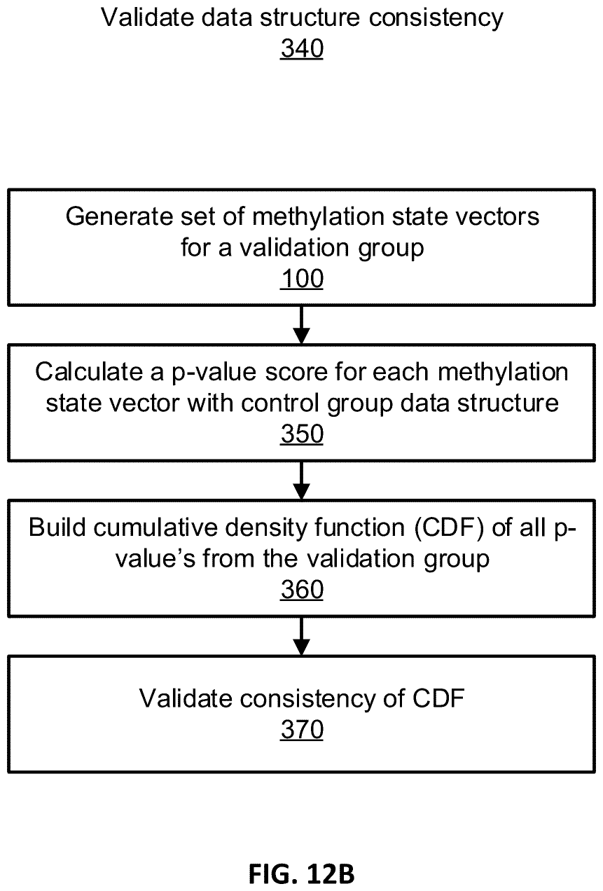

FIG. 12A is a flowchart describing a process 300 of generating a data structure for a healthy control group, according to an embodiment. To create a healthy control group data structure, the analytics system obtains information related to methylation status of a plurality of CpG sites on sequence reads derived from a plurality of DNA molecules or fragments from a plurality of healthy subjects. The method provided herein for creating a healthy control group data structure can be performed similarly for subjects with cancer, subjects with cancer of a TOO, subjects with a known cancer type, or subjects with another known disease state. A methylation state vector is generated for each DNA molecule or fragment, for example via the process 100.

The analytics system subdivides 310 the methylation state vector of each DNA fragment into strings of CpG sites. In one embodiment, the analytics system subdivides 310 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 would result in 4 strings of length 4, 5 strings of length 3, 6 strings of length 2, and 7 strings of length 1. If the methylation state vector resulting from a DNA fragment is shorter than or the same length as the specified string length, then the methylation state vector may be converted into a single string containing all CpG sites of the vector.

The analytics system tallies 320 the strings by counting, for each possible CpG site and possibility of methylation states in the vector, the number of strings present in the control group having the specified CpG site as the first CpG site in the string and having that possibility of methylation states. For a string length of three at a given CpG site, there are 2\hat{ }3 or 8 possible string configurations. For each CpG site, the analytics system tallies 320 how many occurrences of each possible methylation state vector appear in the control group. This may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site in the reference genome. The analytics system creates 330 a data structure storing the tallied counts for each starting CpG site and string possibility at each starting CpG.

There are several benefits to setting an upper limit on string length. First, depending on the maximum length for a string, the size of the data structure created by the analytics system can dramatically increase in size. For instance, a maximum string length of 4 means that there are at most 2∝numbers to tally at every CpG. Increasing the maximum string length to 5 doubles the possible number of methylation states to tally. Reducing string size helps reduce the computational and data storage burden of the data structure. In some embodiments, the string size is 3. In some embodiments, the string size is 4. A second reason to limit the maximum string length is to avoid overfitting downstream models. Calculating probabilities based on long strings of CpG sites can be problematic if the long CpG strings do not have a strong biological effect on the outcome (e.g., predictions of anomalousness that predictive of the presence of cancer), as it requires a significant amount of data that may not be available, and would thus be too sparse for a model to perform appropriately. For example, calculating a probability of anomalousness/cancer conditioned on the prior 100 CpG sites would require counts of strings in the data structure of length 100, ideally some matching exactly the prior 100 methylation states. If only sparse counts of strings of length 100 are available, there will be insufficient data to determine whether a given string of length of 100 in a test sample is anomalous or not.

### Validation of Data Structure

Once the data structure has been created, the analytics system may seek to validate 340 the data structure and/or any downstream models making use of the data structure.

This first type of validation ensures that potential cancerous samples are removed from the healthy control group so as to not affect the control group's purity. This type of validation checks consistency within the control group's data structure. For example, the healthy control group may contain a sample from an individual with an undiagnosed cancer that contains a plurality of anomalously methylated fragments. The analytics system may perform various calculations to determine whether to exclude data from a subject with apparently undiagnosed cancer.

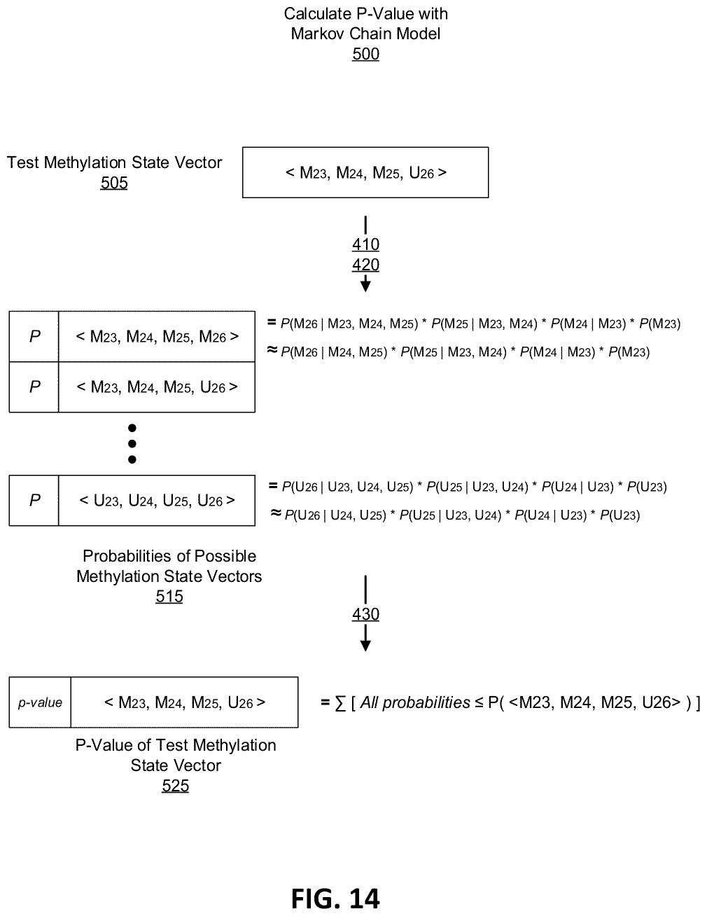

A second type of validation checks the probabilistic model used to calculate p-values with the counts from the data structure itself (i.e., from the healthy control group). A process for p-value calculation is described below in conjunction with FIG. 14. Once the analytics system generates a p-value for the methylation state vectors in the validation group, the analytics system builds a cumulative density function (CDF) with the p-values. With the CDF, the analytics system may perform various calculations on the CDF to validate the control group's data structure. One test uses the fact that the CDF should ideally be at or below an identity function, such that CDF(x)≤x. On the converse, being above the identity function reveals some deficiency within the probabilistic model used for the control group's data structure. For example, if 1/100 of fragments have a p-value score of 1/1000 meaning CDF( 1/1000)= 1/100> 1/1000, then the second type of validation fails indicating an issue with the probabilistic model. See e.g., U.S. application Ser. No. 16/352,602, published as U.S. Publ. No. 2019/0287652, which is hereby incorporated by reference in its entirety.

A third type of validation uses a healthy set of validation samples separate from those used to build the data structure. This tests if the data structure is properly built and the model works. An exemplary process for carrying out this type of validation is described below in conjunction with FIG. 12B. The third type of validation can quantify how well the healthy control group generalizes the distribution of healthy samples. If the third type of validation fails, then the healthy control group does not generalize well to the healthy distribution.

A fourth type of validation tests with samples from a non-healthy validation group. The analytics system calculates p-values and builds the CDF for the non-healthy validation group. With a non-healthy validation group, the analytics system expects to see the CDF(x)>× for at least some samples or, stated differently, the converse of what was expected in the second type of validation and the third type of validation with the healthy control group and the healthy validation group. If the fourth type of validation fails, then this is indicative that the model is not appropriately identifying the anomalousness that it was designed to identify.

FIG. 12B is a flowchart describing the additional step 340 of validating the data structure for the control group of FIG. 12A, according to an embodiment. In this embodiment of the step 340 of validating the data structure, the analytics system performs the fourth type of validation test as described above which utilizes a validation group with a supposedly similar composition of subjects, samples, and/or fragments as the control group. For example, if the analytics system selected healthy subjects without cancer for the control group, then the analytics system also uses healthy subjects without cancer in the validation group.

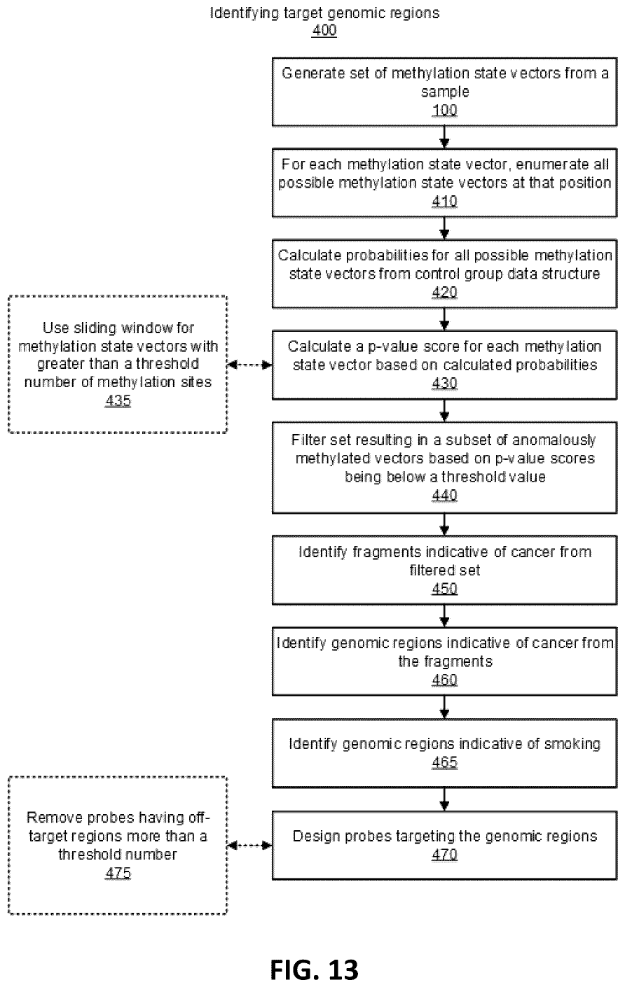

The analytics system takes the validation group and generates 100 a set of methylation state vectors as described in FIG. 12A. The analytics system performs a p-value calculation for each methylation state vector from the validation group. The p-value calculation process will be further described in conjunction with FIGS. 13-14. For each possible methylation state vector, the analytics system calculates a probability from the control group's data structure. Once the probabilities are calculated for the possibilities of methylation state vectors, the analytics system calculates 350 a p-value score for that methylation state vector based on the calculated probabilities. The p-value score represents an expectedness of finding that specific methylation state vector and other possible methylation state vectors having even lower probabilities in the control group. A low p-value score, thereby, generally corresponds to a methylation state vector which is relatively unexpected in comparison to other methylation state vectors within the control group, whereas a high p-value score generally corresponds to a methylation state vector which is relatively more expected in comparison to other methylation state vectors found in the control group. Once the analytics system generates a p-value score for the methylation state vectors in the validation group, the analytics system builds 360 a cumulative density function (CDF) with the p-value scores from the validation group. The analytics system validates 370 consistency of the CDF as described above in the fourth type of validation tests.

### Anomalously Methylated Fragments

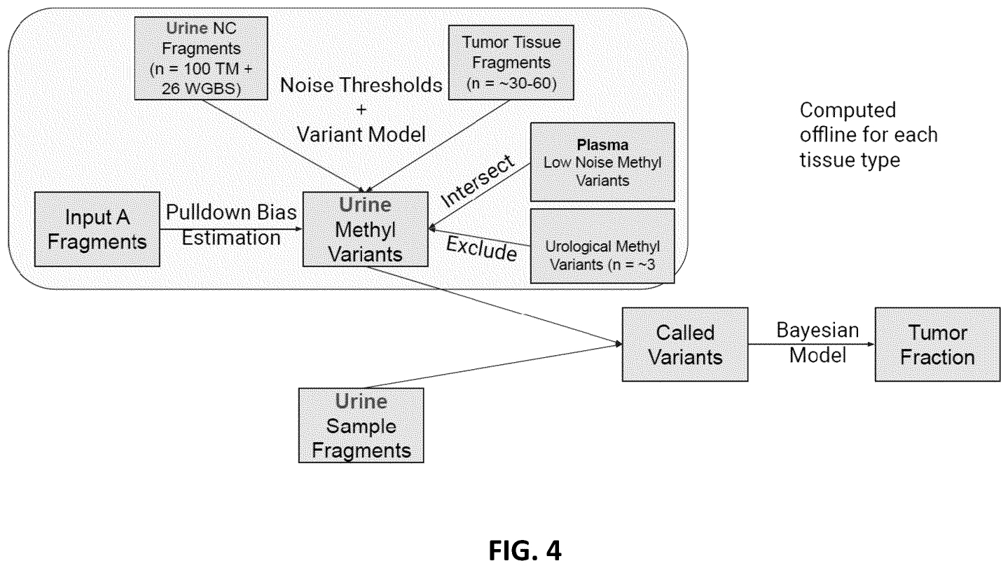

Anomalously methylated fragments having abnormal methylation patterns in cancer patient samples, subjects with cancer of a TOO, subjects with a known cancer type, or subjects with another known disease state, are selected as target genomic regions, according to an embodiment as outlined in FIG. 13. Exemplary processes of selected anomalously methylated fragments 440 are visually illustrated in FIG. 14 and is further described below the description of FIG. 4. In process 400, the analytics system generates 100 methylation state vectors from cfDNA fragments of the sample. The analytics system handles each methylation state vector as follows.

For a given methylation state vector, the analytics system enumerates 410 all possibilities of methylation state vectors having the same starting CpG site and same length (i.e., set of CpG sites) in the methylation state vector. As each methylation state may be methylated or unmethylated there are only two possible states at each CpG site, and thus the count of distinct possibilities of methylation state vectors depends on a power of 2, such that a methylation state vector of length n would be associated with 2ⁿ possibilities of methylation state vectors.

The analytics system calculates 420 the probability of observing each possibility of methylation state vector for the identified starting CpG site/methylation state vector length by accessing the healthy control group data structure. In one embodiment, calculating the probability of observing a given possibility uses a Markov chain probability to model the joint probability calculation which will be described in greater detail with respect to FIG. 14 below. In other embodiments, calculation methods other than Markov chain probabilities are used to determine the probability of observing each possibility of methylation state vector.

The analytics system calculates 430 a p-value score for the methylation state vector using the calculated probabilities for each possibility. In one embodiment, this includes identifying the calculated probability corresponding to the possibility that matches the methylation state vector in question. Specifically, this is the possibility having the same set of CpG sites, or similarly the same starting CpG site and length as the methylation state vector. The analytics system sums the calculated probabilities of any possibilities having probabilities less than or equal to the identified probability to generate the p-value score.

This p-value represents the probability of observing the methylation state vector of the fragment or other methylation state vectors even less probable in the healthy control group. A low p-value score, thereby, generally corresponds to a methylation state vector which is rare in a healthy subject, and which causes the fragment to be labeled abnormally methylated, relative to the healthy control group. A high p-value score generally relates to a methylation state vector is expected to be present, in a relative sense, in a healthy subject. If the healthy control group is a non-cancerous group, for example, a low p-value indicates that the fragment is abnormally methylated relative to the non-cancer group, and therefore possibly indicative of the presence of cancer in the test subject.

As above, the analytics system calculates p-value scores for each of a plurality of methylation state vectors, each representing a cfDNA fragment in the test sample. To identify which of the fragments are abnormally methylated, the analytics system may filter 440 the set of methylation state vectors based on their p-value scores. In one embodiment, filtering is performed by comparing the p-values scores against a threshold and keeping only those fragments below the threshold. This threshold p-value score could be on the order of 0.1, 0.01, 0.001, 0.0001, or similar.

### P-Value Score Calculation

FIG. 14 is an illustration 500 of an example p-value score calculation, according to an embodiment. To calculate a p-value score given a test methylation state vector 505, the analytics system takes that test methylation state vector 505 and enumerates 410 possibilities of methylation state vectors. In this illustrative example, the test methylation state vector 505 is <M₂₃, M₂₄, M₂₅, U₂₆>. As the length of the test methylation state vector 505 is 4, there are 2∝possibilities of methylation state vectors encompassing CpG sites 23-26. In a generic example, the number of possibilities of methylation state vectors is 2\hat{ }n, where n is the length of the test methylation state vector or alternatively the length of the sliding window (described further below).

The analytics system calculates 420 probabilities 515 for the enumerated possibilities of methylation state vectors. As methylation is conditionally dependent on methylation status of nearby CpG sites, one way to calculate the probability of observing a given methylation state vector possibility is to use Markov chain model. Generally, a methylation state vector such as <S₁, S₂, . . . , Sₙ>, where S denotes the methylation state whether methylated (denoted as M), unmethylated (denoted as U), or indeterminate (denoted as I), has a joint probability that can be expanded using the chain rule of probabilities as:

$$P ( 〈 {S}_{1} , {S}_{2} , … , {S}_{n} 〉 ) = P ( {S}_{n} \vert {S}_{1} , … , {S}_{n - 1} 〉 ) * P ( {S}_{n - 1} \vert {S}_{1} , … , {S}_{n - 2} 〉 ) * … * P ( {S}_{2} \vert {S}_{1} ) * P ( {S}_{1} ) \qquad ( 1 )$$

Markov chain model can be used to make the calculation of the conditional probabilities of each possibility more efficient. In one embodiment, the analytics system selects a Markov chain order k which corresponds to how many prior CpG sites in the vector (or window) to consider in the conditional probability calculation, such that the conditional probability is modeled as P(Sₙ|S₁, . . . , Sₙ₋₁)˜P(Sₙ|Sₙ₋ₖ₋₂, . . . , Sₙ₋₁).

To calculate each Markov modeled probability for a possibility of methylation state vector, the analytics system accesses the control group's data structure, specifically the counts of various strings of CpG sites and states. To calculate P(Mₙ|Sₙ₋ₖ₋₂, . . . , Sₙ₋₁), the analytics system takes a ratio of the stored count of the number of strings from the data structure matching<Sₙ₋ₖ₋₂, . . . , Sₙ₋₁, Mₙ> divided by the sum of the stored count of the number of strings from the data structure matching<Sₙ₋ₖ₋₂, . . . , Sₙ₋₁, Mₙ> and <Sₙ₋ₖ₋₂, . . . , Sₙ₋₁, Uₙ>. Thus, P(Mₙ|Sₙ₋ₖ₋₂, . . . , Sₙ₋₁), is calculated ratio having the form:

$$\frac{\# of 〈 {S}_{n - k - 2} , … , {S}_{n - 1} , {M}_{n} 〉}{\# of 〈 {S}_{n - k - 2} , … , {S}_{n - 1} , {M}_{n} 〉 + \# of 〈 {S}_{n - k - 2} , … , {S}_{n - 1} , {U}_{n} 〉} \qquad ( 2 )$$

The calculation may additionally implement a smoothing of the counts by applying a prior distribution. In one embodiment, the prior distribution is a uniform prior as in Laplace smoothing. As an example of this, a constant is added to the numerator and another constant (e.g., twice the constant in the numerator) is added to the denominator of the above equation. In other embodiments, an algorithmic technique such as Knesser-Ney smoothing is used.

In the illustration, the above denoted formulas are applied to the test methylation state vector 505 covering sites 23-26. Once the calculated probabilities 515 are completed, the analytics system calculates 430 a p-value score 525 that sums the probabilities that are less than or equal to the probability of possibility of methylation state vector matching the test methylation state vector 505.

In one embodiment, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities allows for efficient calculation of p-value scores without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

### Sliding Window

In one embodiment, the analytics system uses 435 a sliding window to determine possibilities of methylation state vectors and calculate p-values. Rather than enumerating possibilities and calculating p-values for entire methylation state vectors, the analytics system enumerates possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window identifies the sequential set of CpG sites from the vector within the window starting from the first CpG site in the vector. The analytic system calculates a p-value score for the window including the first CpG site. The analytics system then “slides” the window to the second CpG site in the vector, and calculates another p-value score for the second window. Thus, for a window size l and methylation vector length m, each methylation state vector will generate m−l+1 p-value scores. After completing the p-value calculations for each portion of the vector, the lowest p-value score from all sliding windows is taken as the overall p-value score for the methylation state vector. In another embodiment, the analytics system aggregates the p-value scores for the methylation state vectors to generate an overall p-value score.

Using the sliding window helps to reduce the number of enumerated possibilities of methylation state vectors and their corresponding probability calculations that would otherwise need to be performed. Example probability calculations are shown in FIG. 14, but generally the number of possibilities of methylation state vectors increases exponentially by a factor of 2 with the size of the methylation state vector. To give a realistic example, it is possible for fragments to have upwards of 54 CpG sites. Instead of computing probabilities for 2\hat{ }54 (˜1.8×10\hat{ }16) possibilities to generate a single p-value, the analytics system can instead use a window of size 5 (for example) which results in 50 p-value calculations for each of the 50 windows of the methylation state vector for that fragment. Each of the 50 calculations enumerates 2\hat{ }5 (32) possibilities of methylation state vectors, which total results in 50×2\hat{ }5 (1.6×10\hat{ }3) probability calculations. This results in a vast reduction of calculations to be performed, with no meaningful hit to the accurate identification of anomalous fragments. This additional step can also be applied when validating 340 the control group with the validation group's methylation state vectors.

### Identifying Fragments Indicative of Cancer

The analytics system identifies 450 DNA fragments indicative of cancer from the filtered set of anomalously methylated fragments.

### Hypomethylated and Hypermethylated Fragments

According to a first method, the analytics system may identify DNA fragments that are deemed hypomethylated or hypermethylated as fragments indicative of cancer from the filtered set of anomalously methylated fragments. Hypomethylated and hypermethylated fragments can be defined as fragments of a certain length of CpG sites (e.g., more than 3, 4, 5, 6, 7, 8, 9, 10, etc.) with a high percentage of methylated CpG sites (e.g., more than 80%, 85%, 90%, or 95%, or any other percentage within the range of 50%-100%) or a high percentage of unmethylated CpG sites (e.g., more than 80%, 85%, 90%, or 95%, or any other percentage within the range of 50%-100%).

### Probabilistic Models

According to a method described herein, the analytics system identifies fragments indicative of cancer utilizing probabilistic models of methylation patterns fitted to each cancer type and non-cancer type. The analytics system calculates log-likelihood ratios for a sample using DNA fragments in the genomic regions considering the various cancer types with the fitted probabilistic models for each cancer type and non-cancer type. The analytics system may determine a DNA fragment to be indicative of cancer based on whether at least one of the log-likelihood ratios considered against the various cancer types is above a threshold value.

In one embodiment of partitioning the genome, the analytics system partitions the genome into regions by multiple stages. In a first stage, the analytics system separates the genome into blocks of CpG sites. Each block is defined when there is a separation between two adjacent CpG sites that meets and/or exceeds some threshold, e.g., greater than 200 bp, 300 bp, 400 bp, 500 bp, 600 bp, 700 bp, 800 bp, 900 bp, or 1,000 bp. From each block, the analytics system subdivides at a second stage each block into regions of a certain length, e.g., 500 bp, 600 bp, 700 bp, 800 bp, 900 bp, 1,000 bp, 1,100 bp, 1,200 bp, 1,300 bp, 1,400 bp, or 1,500 bp. The analytics system may further overlap adjacent regions by a percentage of the length, e.g., 10%, 20%, 30%, 40%, 50%, or 60%, or 10% or more, 20% or more, 30% or more, 40% or more, 50% or more, or 60% or more.

The analytics system analyzes sequence reads derived from DNA fragments for each region. The analytics system may process samples from tissue and/or high-signal cfDNA. High-signal cfDNA samples may be determined by a binary classification model, by cancer stage, or by another metric.

For each cancer type and non-cancer, the analytics system fits a separate probabilistic model for fragments. In one example, each probabilistic model is mixture model comprising a combination of a plurality of mixture components with each mixture component being an independent-sites model where methylation at each CpG site is assumed to be independent of methylation statuses at other CpG sites.

In alternate embodiments, calculation is performed with respect to each CpG site. Specifically, a first count is determined that is the number of cancerous samples (cancer_count) that include an anomalously methylated DNA fragment overlapping that CpG, and a second count is determined that is the total number of samples containing fragments overlapping that CpG (total) in the set. Genomic regions can be selected based on the numbers, for example, based on criteria positively correlated to the number of cancerous samples (cancer_count) that include a DNA fragment overlapping that CpG, and inversely correlated to the total number of samples containing fragments overlapping that CpG (total) in the set.

In some embodiments, cancer of various types having different TOO are selected from bladder cancer, urothelial cancer, kidney cancer, and prostate cancer. In some embodiments, the cancer is a bladder cancer. In some embodiments, the cancer is a urothelial cancer. In some embodiments, the cancer is a kidney cancer. In some embodiments, the cancer is a prostate cancer.

In some embodiments, various cancer types can be classified and labeled using classification methods available in the art, such as the International Classification of Diseases for Oncology (ICD-O-3) (codes.iarc.fr) or the Surveillance, Epidemiology, and End Results Program (SEER) (seer.cancer.gov). In other embodiments, cancer types are classified in three orthogonal codes, (i) topographical codes, (ii) morphological codes, or (iii) behavioral codes. Under behavioral codes, benign tumor is 0, uncertain behavior is 1, carcinoma in situ is 2, malignant, primary site is 3 and malignant, metastatic site is 6.

In some embodiments, a cancer TOO can be selected from a group defined by the guideline that will be used to stage a detected cancer. For example, the reference, Amin, M. B., Edge, S., Greene, F., Byrd, D. R., Brookland, R. K., Washington, M. K., Gershenwald, J. E., Compton, C. C., Hess, K. R., Sullivan, D. C., Jessup, J. M., Brierley, J. D., Gaspar, L. E., Schilsky, R. L., Balch, C. M., Winchester, D. P., Asare, E. A., Madera, M., Gress, D. M., Meyer, L. R. (Eds.), AJCC Cancer Staging Manual, 8th edition, Springer, 2017, identifies groups of different cancers that are staged together following standard guidelines. Staging is typically a next step in cancer management following its detection and diagnosis.

The analytics system can further calculate log-likelihood ratios (“R”) for a fragment indicating a likelihood of the fragment being indicative of cancer considering the various cancer types with the fitted probabilistic models for each cancer type and non-cancer type, or for a cancer TOO. The two probabilities may be taken from probabilistic models fitted for each of the cancer types and the non-cancer type, the probabilistic models defined to calculate a likelihood of observing a methylation pattern on a fragment given each of the cancer types and the non-cancer type. For example, the probabilistic models may be defined fitted for each of the cancer types and the non-cancer type.

### Selection of Genomic Regions Indicative of Cancer

In some embodiments, the analytics system can identify 460 genomic regions indicative of cancer. To identify these informative regions, the analytics system calculates an information gain for each genomic region or more specifically each CpG site that describes an ability to distinguish between various outcomes.

A method for identifying genomic regions capable of distinguishing between cancer type and non-cancer type utilizes a trained classification model that can be applied on the set of anomalously methylated DNA molecules or fragments corresponding to or derived from a cancerous or non-cancerous group. The trained classification model can be trained to identify any condition of interest that can be identified from the methylation state vectors.

In one embodiment, the trained classification model is a binary classifier trained based on methylation states for cfDNA fragments or genomic sequences obtained from a subject cohort with cancer or a cancer TOO, and a healthy subject cohort without cancer, and is then used to classify a test subject probability of having cancer, a cancer TOO, or not having cancer, based on anomalously methylation state vectors. In other embodiments, different classifiers may be trained using subject cohorts known to have particular cancer (e.g., bladder, urothelial, prostate, kidney, etc.); known to have cancer of particular TOO where the cancer is believed to originate; or known to have different stages of particular cancer (e.g., bladder, urothelial, prostate, kidney, etc.). In these embodiments, different classifiers may be trained using sequence reads obtained from samples enriched for tumor cells from subject cohorts known to have particular cancer (e.g., bladder, urothelial, prostate, kidney, etc.). Each genomic region's ability to distinguish between cancer type and non-cancer type in the classification model is used to rank the genomic regions from most informative to least informative in classification performance. The analytics system may identify genomic regions from the ranking according to information gain in classification between non-cancer type and cancer type.

Computing Information Gain from Hypomethylated and Hypermethylated Fragments Indicative of Cancer

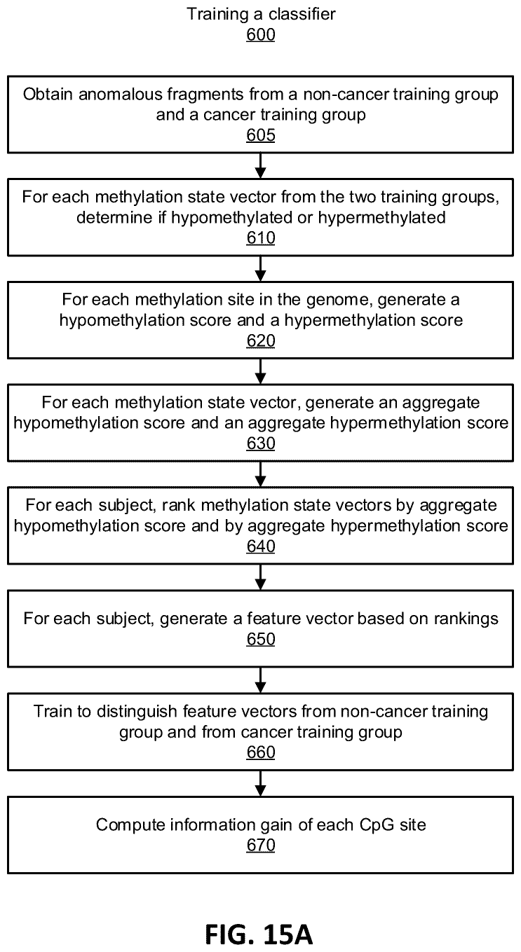

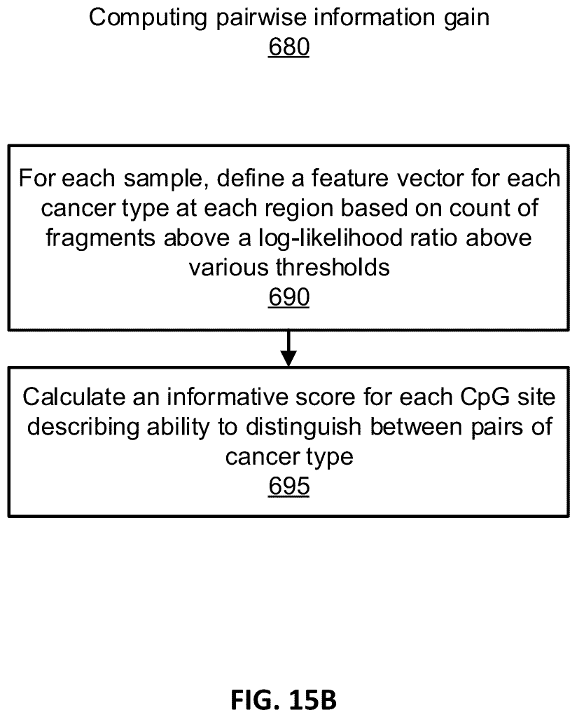

With fragments indicative of cancer, the analytics system may train a classifier according to a process 600 illustrated in FIG. 15A, according to an embodiment. The process 600 accesses two training groups of samples—a non-cancer group and a cancer group—and obtains 605 a non-cancer set of methylation state vectors and a cancer set of methylation state vectors comprising anomalously methylated fragments, e.g., via step 440 from the process 400.

The analytics system determines 610, for each methylation state vector, whether the methylation state vector is indicative of cancer. Here, fragments indicative of cancer may be defined as hypermethylated or hypomethylated fragments determined if at least some number of CpG sites have a particular state (methylated or unmethylated, respectively) and/or have a threshold percentage of sites that are the particular state (again, methylated or unmethylated, respectively). In one example, cfDNA fragments are identified as hypomethylated or hypermethylated, respectively, if the fragment overlaps at least 5 CpG sites, and at least 80%, 90%, or 100% of its CpG sites are methylated or at least 80%, 90%, or 100% are unmethylated.

In an alternate embodiment, the analytics system considers portions of the methylation state vector and determines whether the portion is hypomethylated or hypermethylated, and may distinguish that portion to be hypomethylated or hypermethylated. This alternative resolves missing methylation state vectors which are large in size but contain at least one region of dense hypomethylation or hypermethylation. This process of defining hypomethylation and hypermethylation can be applied in step 450 of FIG. 13. In another embodiment, the fragments indicative of cancer may be defined according to likelihoods outputted from trained probabilistic models.

In one embodiment, the analytics system generates 620 a hypomethylation score (P_{hypo}) and a hypermethylation score (P_{hyper}) per CpG site in the genome. To generate either score at a given CpG site, the classifier takes four counts at that CpG site—(1) count of (methylations state) vectors of the cancer set labeled hypomethylated that overlap the CpG site; (2) count of vectors of the cancer set labeled hypermethylated that overlap the CpG site; (3) count of vectors of the non-cancer set labeled hypomethylated that overlap the CpG site; and (4) count of vectors of the non-cancer set labeled hypermethylated that overlap the CpG site. Additionally, the process may normalize these counts for each group to account for variance in group size between the non-cancer group and the cancer group. In alternative embodiments wherein fragments indicative of cancer are more generally used, the scores may be more broadly defined as counts of fragments indicative of cancer at each genomic region and/or CpG site.

In one embodiment, to generate 620 the hypomethylation score at a given CpG site, the process takes a ratio of (1) over (1) summed with (3). Similarly, the hypermethylation score is calculated by taking a ratio of (2) over (2) and (4). Additionally, these ratios may be calculated with an additional smoothing technique as discussed above. The hypomethylation score and the hypermethylation score relate to an estimate of cancer probability given the presence of hypomethylation or hypermethylation of fragments from the cancer set.

The analytics system generates 630 an aggregate hypomethylation score and an aggregate hypermethylation score for each anomalous methylation state vector. The aggregate hyper and hypo methylation scores are determined based on the hyper and hypo methylation scores of the CpG sites in the methylation state vector. In one embodiment, the aggregate hyper and hypo methylation scores are assigned as the largest hyper and hypo methylation scores of the sites in each state vector, respectively. However, in alternate embodiments, the aggregate scores could be based on means, medians, or other calculations that use the hyper/hypo methylation scores of the sites in each vector.

The analytics system ranks 640 all of that subject's methylation state vectors by their aggregate hypomethylation score and by their aggregate hypermethylation score, resulting in two rankings per subject. The process selects aggregate hypomethylation scores from the hypomethylation ranking and aggregate hypermethylation scores from the hypermethylation ranking. With the selected scores, the classifier generates 650 a single feature vector for each subject. In one embodiment, the scores selected from either ranking are selected with a fixed order that is the same for each generated feature vector for each subject in each of the training groups. As an example, in one embodiment the classifier selects the first, the second, the fourth, and the eighth aggregate hyper methylation score, and similarly for each aggregate hypo methylation score, from each ranking and writes those scores in the feature vector for that subject.

The analytics system trains 660 a binary classifier to distinguish feature vectors between the cancer and non-cancer training groups. Generally, any one of a number of classification techniques may be used. In one embodiment the classifier is a non-linear classifier. In a specific embodiment, the classifier is a non-linear classifier utilizing a L2-regularized kernel logistic regression with a Gaussian radial basis function (RBF) kernel.

Specifically, in one embodiment, the number of non-cancer samples or different cancer type(s) (nₒₜₕₑᵣ) and the number of cancer samples or cancer type(s) (n_{cancer}) having an anomalously methylated fragment overlapping a CpG site are counted. Then the probability that a sample is cancer is estimated by a score (“S”) that positively correlates to n_{cancer} and inversely correlated to nₒₜₕₑᵣ. The score can be calculated using the equation: (n_{cancer}+1)/(n_{cancer}+nₒₜₕₑᵣ+2) or (n_{cancer})/(n_{cancer}+nₒₜₕₑᵣ). The analytics system computes 670 an information gain for each cancer type and for each genomic region or CpG site to determine whether the genomic region or CpG site is indicative of cancer. The information gain is computed for training samples with a given cancer type compared to all other samples. For example, two random variables ‘anomalous fragment’ (‘AF’) and ‘cancer type’ (‘CT’) are used. In on embodiment, AF is a binary variable indicating whether there is an anomalous fragment overlapping a given CpG site in a given samples as determined for the anomaly score/feature vector above. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site.

For a given cancer type, the analytics system uses this information to rank CpG sites based on how cancer specific they are. This procedure is repeated for all cancer types under consideration. If a particular region is commonly anomalously methylated in training samples of a given cancer but not in training samples of other cancer types or in healthy training samples, then CpG sites overlapped by those anomalous fragments will tend to have high information gains for the given cancer type. The ranked CpG sites for each cancer type are greedily added (selected) to a selected set of CpG sites based on their rank for use in the cancer classifier.

Computing Pairwise Information Gain from Fragments Indicative of Cancer Identified from Probabilistic Models

With fragments indicative of cancer identified according to the second method described herein, the analytics may identify genomic regions according to the process 680 in FIG. 15B. The analytics system defines 690 a feature vector for each sample, for each region, for each cancer type by a count of DNA fragments that have a calculated log-likelihood ratio that the fragment is indicative of cancer above a plurality of thresholds, wherein each count is a value in the feature vector. In one embodiment, the analytics system counts the number of fragments present in a sample at a region for each cancer type with log-likelihood ratios above one or a plurality of possible threshold values. The analytics system defines a feature vector for each sample, by a count of DNA fragments for each genomic region for each cancer type that provides a calculated log-likelihood ratio for the fragment above a plurality of thresholds, wherein each count is a value in the feature vector. The analytics system uses the defined feature vectors to calculate an informative score for each genomic region describing that genomic region's ability to distinguish between each pair of cancer types. For each pair of cancer types, the analytics system ranks regions based on the informative scores. The analytics system may select regions based on the ranking according to informative scores.

The analytics system calculates 695 an informative score for each region describing that region's ability to distinguish between each pair of cancer types. For each pair of distinct cancer types, the analytics system may specify one type as a positive type and the other as a negative type. In one embodiment, a region's ability to distinguish between the positive type and the negative type is based on mutual information, calculated using the estimated fraction of cfDNA samples of the positive type and of the negative type for which the feature would be expected to be non-zero in the final assay, i.e., at least one fragment of that tier that would be sequenced in a targeted methylation assay. Those fractions are estimated using the observed rates at which the feature occurs in healthy cfDNA, and in high-signal cfDNA and/or tumor samples of each cancer type. For example, if a feature occurs frequently in healthy cfDNA, then it will also be estimated to occur frequently in cfDNA of any cancer type and would likely result in a low informative score. The analytics system may choose a certain number of regions for each pair of cancer types from the ranking, e.g., 1024.

In additional embodiments, the analytics system further identifies predominantly hypermethylated or hypomethylated regions from the ranking of regions. The analytics system may load the set of fragments in the positive type(s) for a region that was identified as informative. The analytics system, from the loaded fragments, evaluates whether the loaded fragments are predominantly hypermethylated or hypomethylated. If the loaded fragments are predominately hypermethylated or hypomethylated, the analytics system may select probes corresponding to the predominant methylation pattern. If the loaded fragments are not predominantly hypermethylated or hypomethylated, the analytics system may use a mixture of probes for targeting both hypermethylation and hypomethylation. The analytics system may further identify a minimal set of CpG sites that overlap more than some percentage of the fragments.

In other embodiments, the analytics system, after ranking the regions based on informative scores, labels each region with the lowest informative ranking across all pairs of cancer types. For example, if a region was the 10th-most-informative region for distinguishing kidney from bladder, and the 5th-most-informative for distinguishing kidney from prostate, then it would be given an overall label of “5”. The analytics system may design probes starting with the lowest-labeled regions while adding regions to the panel, e.g., until the panel's size budget has been exhausted.

### Off-Target Genomic Regions

In some embodiments, probes targeting selected genomic regions are further filtered 475 based on the number of similar or identical off-target sequences in the genome. This is for screening probes that pull down too many cfDNA fragments corresponding to, or derived from, off-target genomic regions. Exclusion of probes having many off-target sequences in the genome can be valuable by decreasing off-target rates and increasing target coverage for a given amount of sequencing.

An off-target genomic region is a genomic region that has sufficient homology to a target genomic region, such that DNA molecules or fragments derived from off-target genomic regions are hybridized to and pulled down by a probe designed to hybridize to a target genomic region. An off-target genomic region can comprise a sequence (or a converted sequence of that same region) that aligns to a probe along at least 35 bp, 40 bp, 45 bp, 50 bp, 60 bp, 70 bp, or 80 bp with at least an 80%, 85%, 90%, 95%, or 97% match rate. In one embodiment, an off-target genomic region is a genomic region comprising a sequence (or a converted sequence of that same region) that aligns to a probe along at least 45 bp with at least a 90% match rate. Various methods can be adopted to screen out off-target genomic regions.

Exhaustively searching the genome to find all off-target genomic regions can be computationally challenging. In one embodiment, a k-mer seeding strategy (which can allow one or more mismatches) is combined to local alignment at the seed locations. In this case, exhaustive searching of good alignments can be guaranteed based on k-mer length, number of mismatches allowed, and number of k-mer seed hits at a particular location. This requires doing dynamic programing local alignment at a large number of locations, so this approach is highly optimized to use vector CPU instructions (e.g., AVX2, AVX512) and also can be parallelized across many cores within a machine and also across many machines connected by a network. A person of ordinary skill will recognize that modifications and variations of this approach can be implemented for the purpose of identifying off-target genomic regions.

In some embodiments, probes having sequence homology with off-target genomic regions, or DNA molecules corresponding to, or derived from off-target genomic regions comprising more than a threshold number are excluded (or filtered) from the panel. For example, probes having sequence homology with off-target genomic regions, or DNA molecules corresponding to, or derived from off-target genomic regions from more than 30, more than 25, more than 20, more than 18, more than 15, more than 12, more than 10, or more than 5 off-target regions are excluded.

In some embodiments, probes are divided into 2, 3, 4, 5, 6, or more separate groups depending on the numbers of off-target regions. For example, probes having sequence homology with no off-target regions or DNA molecules corresponding to, or derived from off-target regions are assigned to high-quality group, probes having sequence homology with 1-18 off-target regions or DNA molecules corresponding to, or derived from 1-18 off-target regions, are assigned to low-quality group, and probes having sequence homology with more than 19 off-target regions or DNA molecules corresponding to, or derived from 19 off-target regions, are assigned to poor-quality group. Other cut-off values can be used for the grouping.

In some embodiments, probes in the lowest quality group are excluded. In some embodiments, probes in groups other than the highest-quality group are excluded. In some embodiments, separate panels are made for the probes in each group. In some embodiments, all the probes are put on the same panel, but separate analysis is performed based on the assigned groups.

In some embodiments, a panel comprises a larger number of high-quality probes than the number of probes in lower groups. In some embodiments, a panel comprises a smaller number of poor-quality probes than the number of probes in other group. In some embodiments, more than 95%, 90%, 85%, 80%, 75%, or 70% of probes in a panel are high-quality probes. In some embodiments, less than 35%, 30%, 20%, 10%, 5%, 4%, 3%, 2% or 1% of the probes in a panel are low-quality probes. In some embodiments, less than 5%, 4%, 3%, 2% or 1% of the probes in a panel are poor-quality probes. In some embodiments, no poor-quality probes are included in a panel.

In some embodiments, probes having below 50%, below 40%, below 30%, below 20%, below 10% or below 5% are excluded. In some embodiments, probes having above 30%, above 40%, above 50%, above 60%, above 70%, above 80%, or above 90% are selectively included in a panel.

### Methods of Using a Cancer Assay Panel

In one aspect, methods of using a cancer assay panel (alternatively referred to as a “bait set”) are provided. The cancer assay panel may be any disclosed herein, such as with respect to any of the various aspects. The methods can comprise steps of treating DNA molecules or fragments to convert unmethylated cytosines to uracils (e.g., using bisulfite treatment), applying a cancer panel (as described herein) to the converted DNA molecules or fragments, enriching a subset of converted DNA molecules or fragments that bind to the probes in the panel, and sequencing the enriched DNA fragments. In some embodiments, the sequence reads can be compared to a reference genome (e.g., a human reference genome), allowing for identification of methylation states at a plurality of CpG sites within the DNA molecules or fragments and thus provide information relevant to detection of cancer. In some embodiments, the DNA is cfDNA. In some embodiments, the cfDNA is from a urine sample. In some embodiments, target genomic regions identified as particularly useful in characterizing cfDNA from a urine sample are also useful in characterizing cfDNA in other sample types (e.g., cfDNA from biological fluids, such as blood, serum, or plasma). In some embodiments, information concerning target genomic regions disclosed herein are applied to assays of cfDNA from such other sample types for the detection of cancer.

In one aspect, the present disclosure provides a method of detecting cancer cells in a subject that comprises: (a) capturing converted cell-free DNA (cfDNA) fragments from a urine sample of the subject, or amplification products thereof, wherein (i) the bait oligonucleotide composition comprises a plurality of different bait oligonucleotides and (ii) each bait oligonucleotide of the plurality of different bait oligonucleotides hybridizes to a target sequence of a gene selected from Table 1, wherein the target sequence is at least 25 nucleotides in length; (b) separating bait-bound DNA from unbound DNA; (c) sequencing the separated DNA to produced sequencing reads; and (d) detecting the cancer cells with a trained classifier.

The cell-free DNA fragments may be obtained from a urine sample prepared by any of the methods disclosed herein. In some embodiments, the cell-free nucleic acids are obtained by (i) treating a urine sample to inhibit cell lysis; (ii) separating cfDNA fragments in the treated urine sample from cells in the treated urine sample, thereby producing a purified urine sample comprising the cfDNA fragments; (iii) concentrating the cfDNA fragments in the purified urine sample by passing at least a portion of the purified urine sample through a filter, wherein the concentrating producing a filtrate and a retained urine sample, and wherein the retained urine sample comprises an increased concentration of cfDNA fragments; and (iv) isolating cfDNA fragments from the retained urine sample. In some embodiments, treating the urine sample to inhibit cell lysis comprises contacting the urine sample with one or more preservative reagent, non-limiting examples of which are described herein. In some embodiments, the treating includes treatment with a nuclease inhibitor, a formaldehyde quencher, or both. Non-limiting examples of nuclease inhibitors and formaldehyde quenchers are described herein. In some embodiments, the treatment includes contacting the urine sample with a composition comprising imidazolidinyl urea, EDTA, glycine, or a combination thereof. In some embodiments, the treatment includes contacting the urine sample with a composition comprising sodium azide, EDTA, or a combination thereof. Separating the nucleic acid can comprise centrifugating the treated urine sample to pellet cells. A filter used in preparing the retained urine sample may be substantially impermeable to cell-free nucleic acids, but permeable to salts in the purified urine sample. In some embodiments, the filter comprises a rated molecular weight cutoff of 10 kD, 5 kD, 3 kD, or lower. In some embodiments, the retained urine sample has a concentration that is increased by at least 2-fold, at least 5-fold, at least 10-fold, or at least 15-fold compared to the purified urine sample. In some instances, the retained urine sample has a volume that is at least 50%, at least 75%, or at least 90% lower compared to the volume of the treated urine sample. The volume of the treated urine sample may be 5 mL, 10 mL, 15 mL, 20 mL, 30 mL, 40 mL, 50 mL, or more. The method may further include freezing the retained urine sample. In some embodiments, the treating is completed within 120, 60, or 30 minutes after collection of the urine sample. In some embodiments, separating and concentrating are completed within 1 to 14 days after collection (e.g., within 7 days). In some embodiments, the method further comprises amplifying one or more of the isolated cfDNA fragments.

The bait oligonucleotides used to captured the converted cell-free DNA molecules may be any of the bait oligonucleotides described herein, such as with regard to the various compositions described herein. In some embodiments, each of the plurality of different bait oligonucleotides of a panel is at least 45 nucleotides in length (e.g., at least 60, 75, 80, 90, 100, 110, or 120 nucleotides in length). In some embodiments, each bait oligonucleotide in the plurality of probes is no more than 130, 140, 150, 200, 250, or 300 bases in length. In some embodiments, each bait oligonucleotide is 45 to 300, 60 to 200, or 75 to 150 nucleotides in length. In some embodiments, the bait oligonucleotides are at least 50 nucleotides in length. In some embodiments, the bait oligonucleotides are at least 60 nucleotides in length. In some embodiments, the bait oligonucleotides are at least 75 nucleotides in length. In some embodiments, the indicate length of the bait oligonucleotide is the length that is designed to be complementary to a portion of a target genomic sequence, or a converted DNA molecule thereof.

In some embodiments, the cancer assay panel is designed to target at least 500, 1000, 1500, 5000, 10000, 12500, 15000, 17000, 19000, or more target genomic regions. In some embodiments, the cancer assay panel is designed to target fewer than 25000, 20000, 17000, 15000, 12500, 10000, or fewer target genomic regions. In some embodiments, the cancer assay panel is designed to target 5000 to 30000, 10000 to 25000, 12500 to 20000, or 15000 to 20000 target genomic regions. In some embodiments, the cancer assay panel is designed to target 100 to 500, 500 to 1000, 1500 to 5000, 5000 to 10000 target genomic regions. In some embodiments, the cancer assay panel is designed to target at least 500 target genomic regions. In some embodiments, the cancer assay panel is designed to target at least 1000 target genomic regions. In some embodiments, the cancer assay panel is designed to target at least 10000 target genomic regions. In some embodiments, the cancer assay panel is designed to target at least 15000 target genomic regions. In some embodiments, the cancer assay panel is designed to target fewer than 20000 target genomic regions.

In some embodiments, the bait oligonucleotides are configured to hybridize to converted DNA molecules (e.g., converted cfDNA molecules) corresponding to, or derived from, one or more genomic regions. Accordingly, the bait oligonucleotides can have a sequence different from the targeted genomic region. For example, a DNA containing an unmethylated CpG site can be converted to include UpG instead of CpG by deamination (e.g., by treatment with a cytosine deaminase or bisulfite). As a result, a probe to such a target may be configured to hybridize to a sequence including UpG instead of a naturally-existing unmethylated CpG. Accordingly, a complementary site in the probe to the unmethylated site can comprise CpA instead of CpG, and some probes targeting a hypomethylated site where all methylation sites are unmethylated may have no guanine (G) bases. In some embodiments, at least 3%, 5%, 10%, 15%, or 20% of the probes comprise no CpG sequences. In some embodiments, at least 5% of the probes comprise no CpG sequences. In some embodiments, at least 10% of the probes comprise no CpG sequences.

In some embodiments, the cancer assay panel is used to detect the presence or absence of cancer generally and/or provide a cancer classification such as cancer type, stage of cancer such as I, II, III, or IV, or provide the TOO where the cancer is believed to originate. The panel may include probes targeting genomic regions differentially methylated between general cancerous (pan-cancer) samples and non-cancerous samples, or only in cancerous samples with a specific cancer type (e.g., bladder cancer-specific targets). For example, in some embodiments, a cancer assay panel is designed to include differentially methylated genomic regions based on converted (e.g., bisulfite) sequencing data generated from the cfDNA and/or whole genomic DNA of a set of cancer and non-cancer individuals.

In some embodiments, each of the target genomic regions is differentially methylated in at least one of a plurality of cancer types. In some embodiments, the plurality of cancer types comprises at least 2 cancer types (e.g., at least 2, 3, 3, 4, or more cancer types). In some embodiments, the plurality of cancer types include a urological cancer. In some embodiments, the plurality of cancer types includes one or more of a bladder cancer, a urothelial cancer, a prostate cancer, or a kidney cancer.

Each of the probes (or probe pairs) may be designed to target one or more target genomic regions. The target genomic regions can be selected based on several criteria designed to increase selective enriching of informative cfDNA fragments while decreasing noise and non-specific bindings. Various filtering procedures for determining whether to include a target genomic regions are described herein. In some embodiments, two or more of the filtering procedures described herein are used in combination.

In some embodiments, cancer is detected based on sequencing data using a trained classifier. In some embodiments, the trained classifier detects a number of sequencing reads above a threshold for one or more of the target sequences that are identified as hypermethylated and/or hypomethylated in the cfDNA fragments. In some embodiments, the trained classifier discriminates a subject with cancer from a subject without cancer with a defined specificity. Accordingly, in some embodiments, a sample from a subject having cancer of an unknown type (e.g., an undiagnosed cancer) may be used to identify which of a variety of different types of cancer is likely to be present, and which are not. In some embodiments, the classifier is a binary classifier, a mixture model classifier, or a multilayer perceptron model classifier. In some embodiments, the classifier is a mixture model classifier. In some embodiments, the defined specificity for each of the plurality of cancer types is 0.900 or higher (e.g., at least 0.950, 0.975, 0.980, 0.985, 0.990, 0.995, or higher). In some embodiments, the application of the trained classifier comprises a sensitivity of at least 30% (e.g., at least 40%, 50%, 60%, 70%, 80%, or higher) for each of the plurality of cancer types. In some embodiments, the trained classifier has a sensitivity of at least 30% for determining a likelihood of cancer with a specificity of 0.900 or higher for each of the plurality of cancer types. In some embodiments, the trained classifier has a sensitivity of at least 40% for determining a likelihood of cancer with a specificity of 0.990 or higher for each of the plurality of cancer types.

In some embodiments, the method comprises treating the cfDNA molecules to differentiate methylated nucleotides and an unmethylated nucleotides, thereby generating the converted cfDNA molecules. In some embodiments, the treatment comprises deamination, such as treating the cfDNA molecules with a cytosine deaminase or with bisulfite. In some embodiments, the method comprises treating the cfDNA molecules with bisulfite to generate the converted cfDNA molecules.

In some embodiments, each bait oligonucleotide is conjugated to a solid surface (e.g., a chip or a bead, such as a magnetic or paramagnetic bead) or to a non-nucleotide affinity moiety (e.g., a member of a binding pair). In some embodiments, such conjugation is used to facilitate separation of DNA molecules bound to bait oligonucleotides from unbound DNA molecules. In some embodiments, the affinity moiety is biotin.

In some embodiments, the genomic regions can be selected to have at least 3, 5, or 7 methylation sites. In some embodiments, each target genomic regions comprises at least five methylation sites. In some embodiments, the selected number of methylation sites (e.g., at least 5 methylation sites) are methylation sites that are differentially methylated in at least one type of cancer to be assayed by the panel. In some embodiments, the target genomic region comprises at least 1, 2, 3, 4, 5, 10, 15 or more of the target genomic regions comprising a target sequence from one or more genes selected from Table 1. In some embodiments, the target genomic region comprises a target sequence of a gene selected from TWIST1, EOMES, HOXA9, POU4F2, and ZNF154.

In some embodiments, the method further comprises diagnosing a cancer in the subject. In some embodiments, the method further comprises selecting a treatment for the identified cancer type. In some embodiments, the method further comprises treating the subject for the cancer. In some embodiments, the cancer types comprise bladder cancer, urothelial cancer, prostate cancer, or kidney cancer. The particular mode of treatment may depend on one or more of various factors, such as the particular type of cancer detected, the cancer TOO, location, and stage. Non-limiting examples of treatment include surgical resection, radiation therapy, chemotherapy, and/or immunotherapy.

### Analysis of Sequence Reads

In some embodiments, the sequence reads may be aligned to a reference genome using various methods to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary alignment map) format may be generated and output for further analysis.

From the sequence reads, the location and methylation state for each of CpG site may be determined based on alignment to a reference genome. Further, a methylation state vector for each fragment may be generated specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). The methylation state vectors may be stored in temporary or persistent computer memory for later use and processing. Further, duplicate reads or duplicate methylation state vectors from a single subject may be removed. In an additional embodiment, it may be determined that a certain fragment has one or more CpG sites that have an indeterminate methylation status. Such fragments may be excluded from later processing or selectively included where downstream data model accounts for such indeterminate methylation statuses.

FIG. 16B is an illustration of the process 100 of FIG. 16A of sequencing a cfDNA fragment to obtain a methylation state vector, according to an embodiment. As an example, the analytics system takes a cfDNA fragment 112. In this example, the cfDNA fragment 112 contains three CpG sites. As shown, the first and third CpG sites of the cfDNA fragment 112 are methylated 114. During the treatment step 120, the cfDNA fragment 112 is converted to generate a converted cfDNA fragment 122. During the treatment 120, the second CpG site which was unmethylated has its cytosine converted to uracil. However, the first and third CpG sites are not converted.

After conversion, a sequencing library 130 is prepared and sequenced 140 generating a sequence read 142. The analytics system aligns 150 the sequence read 142 to a reference genome 144. The reference genome 144 provides the context as to what position in a human genome the fragment cfDNA originates from. In this simplified example, the analytics system aligns 150 the sequence read such that the three CpG sites correlate to CpG sites 23, 24, and 25 (arbitrary reference identifiers used for convenience of description). The analytics system thus generates information both on methylation status of all CpG sites on the cfDNA fragment 112 and which to position in the human genome the CpG sites map. As shown, the CpG sites on sequence read 142 which were methylated are read as cytosines. In this example, the cytosine's appear in the sequence read 142 only in the first and third CpG site which allows one to infer that the first and third CpG sites in the original cfDNA fragment were methylated. The second CpG site is read as a thymine (U is converted to T during the sequencing process), and thus, one can infer that the second CpG site was unmethylated in the original cfDNA fragment. With these two pieces of information, the methylation status and location, the analytics system generates 160 a methylation state vector 152 for the fragment cfDNA 112. In this example, the resulting methylation state vector 152 is <M₂₃, U₂₄, M₂₅>, wherein M corresponds to a methylated CpG site, U corresponds to an unmethylated CpG site, and the subscript numbers correspond to positions of each CpG site in the reference genome.

### Detection of Cancer

Sequence reads obtained by the methods provided herein can be further processed by automated algorithms. For example, the analytics system is used to receive sequencing data from a sequencer and perform various aspects of processing as described herein. The analytics system can be one of a personal computer (PC), a desktop computer, a laptop computer, a notebook, a tablet PC, a mobile device. A computing device can be communicatively coupled to the sequencer through a wireless, wired, or a combination of wireless and wired communication technologies. Generally, the computing device is configured with a processor and memory storing computer instructions that, when executed by the processor, cause the processor to perform steps as described in the remainder of this document. Generally, the amount of genetic data and data derived therefrom is sufficiently large, and the amount of computational power required so great, so as to be impossible to be performed on paper or by the human mind alone.

The clinical interpretation of methylation status of targeted genomic regions is a process that can include classifying the clinical effect of each or a combination of the methylation status and reporting the results in ways that are meaningful to a medical professional. The clinical interpretation can be based on comparison of the sequence reads with database specific to cancer or non-cancer subjects, and/or based on numbers and types of the cfDNA fragments having cancer-specific methylation patterns identified from a sample. In some embodiments, targeted genomic regions are ranked or classified based on their likeness to be differentially methylated in cancer samples, and the ranks or classifications are used in the interpretation process. The ranks and classifications can include (1) the type of clinical effect, (2) the strength of evidence of the effect, and (3) the size of the effect. Various methods for clinical analysis and interpretation of genome data can be adopted for analysis of the sequence reads. In some other embodiments, the clinical interpretation of the methylation states of such differentially methylated regions can be based on machine learning approaches that interpret a current sample based on a classification or regression method that was trained using the methylation states of such differentially methylated regions from samples from cancer and non-cancer patients with known cancer status, cancer type, cancer stage, TOO, etc.

The clinically meaning information can include the presence or absence of cancer generally, presence or absence of certain types of cancers, cancer stage, or presence or absence of other types of diseases. In some embodiments, the information relates to a presence or absence of a urological cancer. In some embodiments, the information relates to a presence or absence of one or more cancer types, selected from the group consisting of bladder cancer, urothelial cancer, kidney cancer, and prostate cancer.

### Cancer Classifier

In some examples, the assay panel described herein can be used with a cancer type classifier that predicts a disease state for a sample, such as a cancer or non-cancer prediction, a tissue of origin prediction, and/or an indeterminate prediction. In some examples, the cancer type classifier can generate features based on sequence reads by taking into account methylated or unmethylated fragments of DNA at certain genomic areas of interest. For instance, if the cancer type classifier determines that a methylation pattern at a fragment resembles that of a certain cancer type, then the cancer type classifier can set a feature for that fragment as 1, and otherwise if no such fragment is present, then the feature can be set as 0. In this way, the cancer type classifier can produce a set of binary features (merely by way of example, 30,000 features) for each sample. Further, in some examples, all or a portion of the set of binary features for a sample can be input into the cancer type classifier to provide a set of probability scores, such as one probability score per cancer type class and for a non-cancer type class. Furthermore, in some examples, the cancer type classifier can incorporate or otherwise be used in conjunction with thresholding to determine whether a sample is to be called as cancer or non-cancer, and/or indeterminate thresholding to reflect confidence in a specific TOO call. Such methods are described further below.

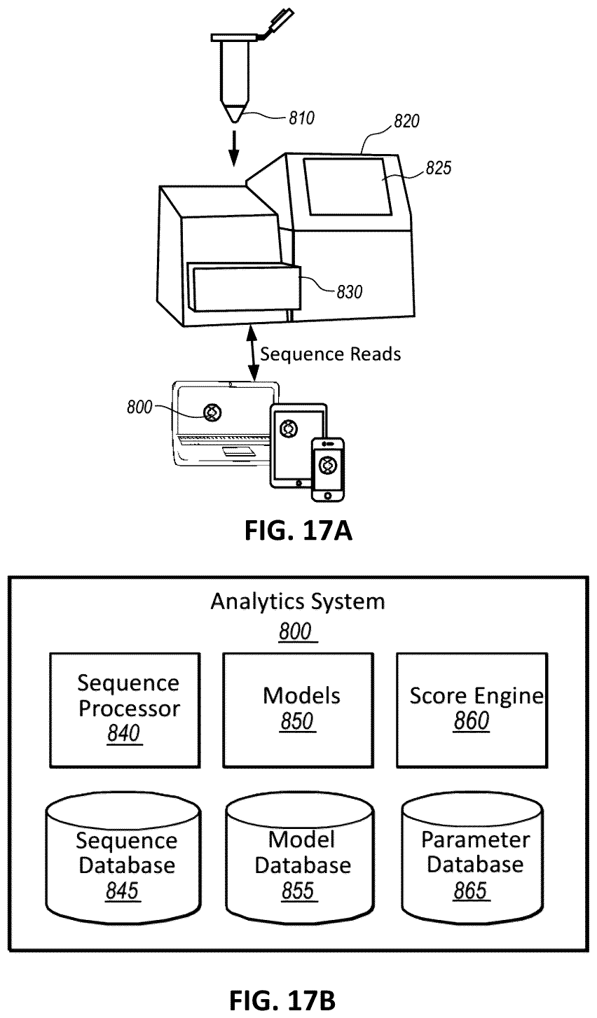

To train the cancer type classifier, the analytics system (e.g., analytics system 800, FIG. 17B) can obtain a set of training samples. In some examples, each training sample includes fragment file(s) (e.g., file containing sequence read data), a label corresponding to a type of cancer (TOO) or non-cancer status of the sample, and/or sex of the individual of the sample. The analytics system can utilize the training set to train the cancer type classifier to predict the disease state of the sample.

In some examples, for training, the analytics system divides the genome (e.g., whole genome) or a subset of the genome (e.g., targeted methylation regions) into regions. Merely by way of example, portions of the genome can be separated into “blocks” of CpGs, whereby a new block begins whenever there is a separation between nearest-neighbor CpGs is at least a minimum separation distance (e.g., at least 500 bp). Further, in some examples, each block can be divided into 1000 bp regions and positioned such that neighboring regions have a certain amount (e.g., 50% or 500 bp) of overlap.

Furthermore, in some examples, the analytics system can split the training set into K subsets or folds to be used in a K-fold cross-validation. In some examples, the folds can be balanced for cancer/non-cancer status, tissue of origin, cancer stage, age (e.g., grouped in 10 yr buckets), and/or smoking status. In some examples, the training set is split into 5 folds, whereby 5 separate classifiers are trained, in each case training on 4/5 of the training samples and using the remaining ⅕ for validation.

During training with the training set, the analytics system can, for each cancer type (and for healthy cfDNA), fit a probabilistic model to the fragments deriving from the samples of that type. As used herein a “probabilistic model” is any mathematical model capable of assigning a probability to a sequence read based on methylation status at one or more sites on the read. During training, the analytics system fits sequence reads derived from one or more samples from subjects having a known disease and can be used to determine sequence reads probabilities indicative of a disease state utilizing methylation information or methylation state vectors. In particular, in some cases, the analytics system determines observed rates of methylation for each CpG site within a sequence read. The rate of methylation represents a fraction or percentage of base pairs that are methylated within a CpG site. The trained probabilistic model can be parameterized by products of the rates of methylation. In general, any known probabilistic model for assigning probabilities to sequence reads from a sample can be used. For example, the probabilistic model can be a binomial model, in which every site (e.g., CpG site) on a nucleic acid fragment is assigned a probability of methylation, or an independent sites model, in which each CpG's methylation is specified by a distinct methylation probability with methylation at one site assumed to be independent of methylation at one or more other sites on the nucleic acid fragment.

In some examples, the probabilistic model is a Markov model, in which the probability of methylation at each CpG site is dependent on the methylation state at some number of preceding CpG sites in the sequence read, or nucleic acid molecule from which the sequence read is derived. See, e.g., U.S. patent application Ser. No. 16/352,602, entitled “Anomalous Fragment Detection and Classification,” and filed Mar. 13, 2019, which is incorporated by reference in its entirety herein and can be used for various embodiments.

In some examples, the probabilistic model is a “mixture model” fitted using a mixture of components from underlying models. For example, in some embodiments, the mixture components can be determined using multiple independent sites models, where methylation (e.g., rates of methylation) at each CpG site is assumed to be independent of methylation at other CpG sites. Utilizing an independent sites model, the probability assigned to a sequence read, or the nucleic acid molecule from which it derives, is the product of the methylation probability at each CpG site where the sequence read is methylated and one minus the methylation probability at each CpG site where the sequence read is unmethylated. In accordance with this example, the analytics system determines rates of methylation of each of the mixture components. The mixture model is parameterized by a sum of the mixture components each associated with a product of the rates of methylation. A probabilistic model Pr of n mixture components can be represented as:

$$Pr ( fragment \vert \{ {β}_{ki} , {f}_{k} \} ) = {\sum }_{k = 1}^{n} {f}_{k} {\prod }_{i} {{β}_{ki}^{{m}_{i}} ( 1 - {β}_{ki} )}^{1 - {m}_{i}}$$

For an input fragment, mᵢ∈{0, 1} represents the fragment's observed methylation status at position i of a reference genome, with 0 indicating unmethylation and 1 indicating methylation. A fractional assignment to each mixture component k is fₖ, where fₖ≥0 and Σₖ₌₁ⁿ fₖ=1. The probability of methylation at position i in a CpG site of mixture component k is βₖᵢ. Thus, the probability of unmethylation is 1−βₖᵢ. The number of mixture components n can be 1, 2, 3, 4, 5, 6, 7, 8, 9, 10, etc.

In some examples, the analytics system fits the probabilistic model using maximum-likelihood estimation to identify a set of parameters {βₖᵢ, fₖ} that maximizes the log-likelihood of all fragments deriving from a disease state, subject to a regularization penalty applied to each methylation probability with regularization strength r. The maximized quantity for N total fragments can be represented as:

$${\sum }_{j}^{N} ln ( Pr ( {fragment}_{j} \vert \{ {β}_{ki} , {f}_{k} \} ) ) + r \cdot ln ( {β}_{ki} ( 1 - {β}_{ki} ) )$$

In some examples, the analytics system performs fits separately for each cancer type and for healthy cfDNA. According to various aspects of the subject disclosure, other means can be used to fit the probabilistic models or to identify parameters that maximize the log-likelihood of all sequence reads derived from the reference samples. For example, in some examples, Bayesian fitting (using e.g., Markov chain Monte Carlo), in which each parameter is not assigned a single value but instead is associated to a distribution, is used. In some examples, gradient-based optimization, in which the gradient of the likelihood (or log-likelihood) with respect to the parameter values is used to step through parameter space towards an optimum, is used. In still some examples, expectation-maximization, in which a set of latent parameters (such as identities of the mixture component from which each fragment is derived) are set to their expected values under the previous model parameters, and then the model's parameters are assigned to maximize the likelihood conditional on the assumed values of those latent variables. The two-step process is then repeated until convergence.

Further, in some examples, the analytics system can generate features for each sample in the training set. For example, for each sample (regardless of label), in each region, for each cancer type, for each fragment, the analytics system can evaluate the log-likelihood ratio R with the fitted probabilistic models according to:

$${R}_{cancer type A} ( fragment ) ≡ ln ( \frac{Pr ( fragment \vert cancer type A}{Pr ( fragment \vert healthy cfDNA )} )$$

Next, for each sample, for each region, for each cancer type, for each of a set of “tier” values, the analytics system can count the number of fragments with Rcancer type>tier and assign those counts as non-negative integer-valued features. For example, the tiers include threshold values of 1, 2, 3, 4, 5, 6, 7, 8, and 9, resulting in each region hosting 9 features per cancer type.

In some examples, the analytics system can select certain features for inclusion in a feature vector for each sample. For example, for each pair of distinct cancer types, the analytics system can specify one type as the “positive type” and the other as the “negative type” and rank the features by their ability to distinguish those types. In some cases, the ranking is based on mutual information calculated by the analytics system. For example, the mutual information can be calculated using the estimated fraction of samples of the positive type and negative type (e.g., cancer types A and B) for which the feature is expected to be nonzero in a resulting assay. For instance, if a feature occurs frequently in healthy cfDNA, the analytics system determines the feature is unlikely to occur frequently in cfDNA associated with various types of cancer. Consequently, the feature can be a weak measure in distinguishing between disease states. In calculating mutual information I, the variable X is a certain feature (e.g., binary) and variable Y represents a disease state, e.g., cancer type A or B:

$$I ( X ; Y ) = {\sum }_{y ∈ Y} {\sum }_{x ∈ X} p ( x , y ) log log ( \frac{p ( x , y )}{p ( x ) p ( y )} )$$

$$I \approx \frac{1}{2} ( p ( 1 \vert A ) \cdot log ( \frac{p ( 1 \vert A )}{\frac{1}{2} ( p ( 1 \vert A ) + p ( 1 \vert B ) )} ) + p ( 1 \vert B ) \cdot log ( \frac{p ( 1 \vert B )}{\frac{1}{2} ( p ( 1 \vert A ) + p ( 1 \vert B ) )} ) )$$

$$p ( 1 \vert A ) = {f}_{A} + {f}_{H} - {f}_{H} {f}_{A}$$

The joint probability mass function of X and Y is p(x, y) and the marginal probability mass functions are p(x) and p(y). The analytics system can assume that feature absence is uninformative and either disease state is equally likely a priori, for example, p(Y=A)=p(Y=B)=0.5. The probability of observing (e.g., in cfDNA) a given binary feature of cancer type A is represented by p(1|A), where f_{A} is the probability of observing the feature in ctDNA samples from tumor (or high-signal cfDNA samples) associated with cancer type A, and f_{H} is the probability of observing the feature in a healthy or non-cancer cfDNA sample.

In some examples, only features corresponding to the positive type are included in the ranking, and only when those features' predicted rate of occurrence is greater in the positive type than in the negative type. For example, if “liver” is the positive type and “breast” is the negative type, then only “liver_x” features are considered, and only if their estimated occurrence in liver cfDNA is greater than their estimated occurrence in breast cfDNA. Further, in some examples, for each region, for each cancer type pair (including non-cancer as a negative type), the analytics system keeps only the best performing tier. Further, in some examples, the analytics system transforms feature values by binarization, whereby any feature value greater than 0 is set to 1, such that all features are either 0 or 1.

In some examples, the analytics system trains a multinomial logistic regression classifier on the training data for a fold, and generates predictions for the held-out data. For example, for each of the K folds, one logistic regression can be trained for each combination of hyperparameters. Such hyperparameters can include L2 penalty and/or topK (e.g., the number of high-ranking regions to keep per tissue type pair (including non-cancer), as ranked by the mutual information procedure outlined above). For each set of hyperparameters, performance is evaluated on the cross-validated predictions of the full training set, and the set of hyperparameters with the best performance is selected for retraining on the full training set. In some examples, the analytics system uses log-loss as a performance metric, whereby the log-loss is calculated by taking the negative logarithm of the prediction for the correct label for each sample, and then summing over samples (i.e., a perfect prediction of 1.0 for the correct label would give a log-loss of 0).

To generate predictions for anew sample, feature values are calculated using the same method described above, but restricted to features (region/positive class combinations) selected under the chosen topK value. Generated features are then used to create a prediction using the logistic regression model trained above.

In some examples, the analytics trains a two-stage classifier. For example, the analytics system trains a binary cancer classifier to distinguish between the labels, cancer and non-cancer, based on the feature vectors of the training samples. In this case, the binary classifier outputs a prediction score indicating the likelihood of the presence or absence of cancer. In another example, the analytics system trains a multiclass cancer classifier to distinguish between many cancer types. In this multiclass cancer classifier, the cancer classifier is trained to determine a cancer prediction that comprises a prediction value for each of the cancer types being classified for. The prediction values can correspond to a likelihood that a given sample has each of the cancer types. For example, the cancer classifier returns a cancer prediction including a prediction value for a urological cancer, and non-cancer. For example, the cancer classifier may return a cancer prediction for a test sample including a prediction score for bladder or urothelial cancer, kidney cancer, prostate cancer and/or no cancer.

The analytics system can train the cancer classifier according to any one of a number of methods. As an example, the binary cancer classifier may be a L2-regularized logistic regression classifier that is trained using a log-loss function. As another example, the multi-cancer (TOO) classifier may be a multinomial logistic regression. In practice, either type of cancer classifier may be trained using other techniques. These techniques are numerous including potential use of kernel methods, machine learning algorithms such as multilayer neural networks, etc. In particular, methods as described in PCT/US2019/022122 and U.S. patent application Ser. No. 16/352,602 which are incorporated by reference in their entireties herein can be used for various embodiments. Still further, in some examples, the TOO classifier is trained only on cancer samples that were successfully called as cancer by the binary classifier, thereby ensuring sufficient cancer signal in the cancer sample. On the other hand, in some examples, the binary classifier is trained on the training samples regardless of TOO.

### Exemplary Sequencer and Analytics System

FIG. 17A is a flowchart of systems and devices for sequencing nucleic acid samples according to one embodiment. This illustrative flowchart includes devices such as a sequencer 820 and an analytics system 800. The sequencer 820 and the analytics system 800 may work in tandem to perform one or more steps in the processes described herein.

In various embodiments, the sequencer 820 receives an enriched nucleic acid sample 810. As shown in FIG. 17A, the sequencer 820 can include a graphical user interface 825 that enables user interactions with particular tasks (e.g., initiate sequencing or terminate sequencing) as well as one more loading stations 830 for loading a sequencing cartridge including the enriched fragment samples and/or for loading necessary buffers for performing the sequencing assays. Therefore, once a user of the sequencer 820 has provided the necessary reagents and sequencing cartridge to the loading station 830 of the sequencer 820, the user can initiate sequencing by interacting with the graphical user interface 825 of the sequencer 820. Once initiated, the sequencer 820 performs the sequencing and outputs the sequence reads of the enriched fragments from the nucleic acid sample 810.

In some embodiments, the sequencer 820 is communicatively coupled with the analytics system 800. The analytics system 800 includes some number of computing devices used for processing the sequence reads for various applications such as assessing methylation status at one or more CpG sites, variant calling or quality control. The sequencer 820 may provide the sequence reads in a BAM file format to the analytics system 800. The analytics system 800 can be communicatively coupled to the sequencer 820 through a wireless, wired, or a combination of wireless and wired communication technologies. Generally, the analytics system 800 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using various methods to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 800 may label a sequence read with one or more genes that align to the sequence read. In one embodiment, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. In one embodiment, the read pair R_1 and R_2 can be assembled into a fragment, and the fragment used for subsequent analysis and/or classification. An output file having SAM (sequence alignment map) format or BAM (binary) format may be generated and output for further analysis.

Referring now to FIG. 17B, FIG. 17B is a block diagram of an analytics system 800 for processing DNA samples according to one embodiment. The analytics system implements one or more computing devices for use in analyzing DNA samples. The analytics system 800 includes a sequence processor 840, sequence database 845, model database 855, models 850, parameter database 865, and score engine 860. In some embodiments, the analytics system 800 performs one or more steps in the processes 300 of FIG. 12A, 340 of FIG. 12B, 400 of FIG. 13, 500 of FIG. 14, 600 of FIG. 15A, or 680 of FIG. 15B and other process described herein.

The sequence processor 840 generates methylation state vectors for fragments from a sample. At each CpG site on a fragment, the sequence processor 840 generates a methylation state vector for each fragment specifying a location of the fragment in the reference genome, a number of CpG sites in the fragment, and the methylation state of each CpG site in the fragment whether methylated, unmethylated, or indeterminate via the process 300 of FIG. 12A. The sequence processor 840 may store methylation state vectors for fragments in the sequence database 845. Data in the sequence database 845 may be organized such that the methylation state vectors from a sample are associated to one another.

Further, multiple different models 850 may be stored in the model database 855 or retrieved for use with test samples. In one example, a model is a trained cancer classifier for determining a cancer prediction for a test sample using a feature vector derived from anomalous fragments. The training and use of the cancer classifier is discussed elsewhere herein. The analytics system 800 may train the one or more models 850 and store various trained parameters in the parameter database 865. The analytics system 800 stores the models 850 along with functions in the model database 855.

During inference, the score engine 860 uses the one or more models 850 to return outputs. The score engine 860 accesses the models 850 in the model database 855 along with trained parameters from the parameter database 865. According to each model, the score engine receives an appropriate input for the model and calculates an output based on the received input, the parameters, and a function of each model relating the input and the output. In some use cases, the score engine 860 further calculates metrics correlating to a confidence in the calculated outputs from the model. In other use cases, the score engine 860 calculates other intermediary values for use in the model.

### Cancer and Treatment Monitoring

In some embodiments, cell-free nucleic acid samples (e.g., cfDNA from a urine sample) may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy. In some embodiments, a first time point in cancer monitoring is before a cancer treatment (e.g., before a resection surgery or a therapeutic intervention), and a second time point is after a cancer treatment (e.g., after a resection surgery or therapeutic intervention), and the method is utilized to monitor the effectiveness of the treatment. For example, if the second likelihood or probability score decreases compared to the first likelihood or probability score, then the treatment is considered to have been successful. However, if the second likelihood or probability score increases compared to the first likelihood or probability score, then the treatment is considered to have not been successful. In some embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment.

Test samples can be obtained from a cancer patient over any desired set of time points and analyzed in accordance with the methods of the invention to monitor a cancer state in the patient. In some embodiments, the first and second time points are separated by an amount of time that ranges from 15 minutes up to 30 years, such as 30 minutes, such as 1, 2, 3, 4, 5, 6, 7, 8, 9, 10, 11, 12, 13, 14, 15, 16, 17, 18, 19, 20, 21, 22, 23, or 24 hours, such as 1, 2, 3, 4, 5, 10, 15, 20, 25 or 30 days, or such as 1, 2, 3, 4, 5, 6, 7, 8, 9, 10, 11, or 12 months, or such as 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or 30 years. In other embodiments, test samples can be obtained from the patient at least once every 3 months, at least once every 6 months, at least once a year, at least once every 2 years, at least once every 3 years, at least once every 4 years, or at least once every 5 years.

### Treatment

In some embodiments, information obtained from any method described herein (e.g., the likelihood or probability score) can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment (e.g., a resection surgery, radiation therapy, chemotherapy, and/or immunotherapy). In some embodiments, information such as a likelihood or probability score can be provided as a readout to a physician or subject.

In one aspect, the method comprises selecting a subject having or being at increased risk of developing a cancer type, and administering a treatment to the subject effective to treat the cancer type, wherein: (a) the selecting comprises identifying the subject as the source of a urine cell-free DNA (cfDNA) sample comprising one or more differentially methylated target genomic regions above a threshold level for the presence of the cancer; (b) the one or more target genomic regions comprise one or more target sequences of one or more genes selected from Table 1; (c) each target sequence is at least 25 nucleotides in length; (d) the cancer is bladder cancer, prostate cancer, or kidney cancer; and (e) the treatment comprises surgical resection, radiation therapy, chemotherapy, immunotherapy, or any combination thereof.

A classifier (as described herein) can be used to determine a likelihood or probability score that a sample feature vector is from a subject that has cancer. In one embodiment, an appropriate treatment (e.g., resection surgery or therapeutic) is prescribed when the likelihood or probability exceeds a threshold (e.g., a level for reference samples from subjects having the cancer). For example, in one embodiment, if the likelihood or probability score is greater than or equal to 60, one or more appropriate treatments are prescribed. In another embodiments, if the likelihood or probability score is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, a cancer log-odds ratio can indicate the effectiveness of a cancer treatment. For example, an increase in the cancer log-odds ratio over time (e.g., at a second, after treatment) can indicate that the treatment was not effective. Similarly, a decrease in the cancer log-odds ratio over time (e.g., at a second, after treatment) can indicate successful treatment. In another embodiment, if the cancer log-odds ratio is greater than 1, greater than 1.5, greater than 2, greater than 2.5, greater than 3, greater than 3.5, or greater than 4, one or more appropriate treatments are prescribed. In some embodiments, the threshold level for the presence of the cancer is determined by a classifier trained on sequencing reads for converted DNA from subjects having the cancer. Non-limiting examples of classifiers are described herein. Classification may be based on one or more target genomic regions, as described herein with regard to various aspects of the present disclosure.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### Computer Systems and Devices

In one aspect, the present disclosure provides a computer system for implementing one or more steps of a method disclosed herein. In another aspect, the present disclosure provides a non-transitory, computer-readable medium, having stored thereon computer-readable instructions for implementing one or more steps of a method disclosed herein.

Methods of the disclosure can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer programs include, by way of example, both general and special purpose microprocessors, and any one or more processors of any kind of digital computer. Generally, a processor will receive instructions and data from a read-only memory or a random access memory, or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including, by way of example, semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through a network by any form or medium of digital data communication, e.g., a communication network. For example, a reference set of data may be stored at a remote location and a computer can communicate across a network to access the reference data set for comparison purposes. In other embodiments, however, a reference data set can be stored locally within the computer, and the computer accesses the reference data set within the CPU for comparison purposes. Examples of communication networks include, but are not limited to, cell networks (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, a data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the disclosure can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the disclosure involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification (RFID) tags or chips, or any other medium that can be used to store the desired information, and which can be accessed by a computing device.

Functions described herein can be implemented using software, hardware, firmware, hardwiring, or combinations of any of these. Any of the software can be physically located at various positions, including being distributed such that portions of the functions are implemented at different physical locations.

As one skilled in the art would recognize as necessary or best-suited for performance of the methods of the disclosure, a computer system for implementing some or all of the described inventive methods can include one or more processors (e.g., a central processing unit (CPU) a graphics processing unit (GPU), or both), main memory and static memory, which communicate with each other via a bus.

A processor will generally include a chip, such as a single core or multi-core chip, to provide a central processing unit (CPU). A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc.

While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present disclosure. These terms shall accordingly be taken to include, but not be limited to, one or more solid-state memories (e.g., subscriber identity module (SIM) card, secure digital card (SD card), micro SD card, or solid-state drive (SSD)), optical and magnetic media, and/or any other tangible storage medium or media.

A computer of the disclosure will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

Additionally, systems of the disclosure can be provided to include reference data. Any suitable genomic data may be stored for use within the system. Examples include, but are not limited to: comprehensive, multi-dimensional maps of the key genomic changes in major types and subtypes of cancer from The Cancer Genome Atlas (TCGA); a catalog of genomic abnormalities from The International Cancer Genome Consortium (ICGC); a catalog of somatic mutations in cancer from COSMIC; the latest builds of the human genome and other popular model organisms; up-to-date reference SNPs from dbSNP; gold standard indels from the 1000 Genomes Project and the Broad Institute; exome capture kit annotations from Illumina, Agilent, Nimblegen, and Ion Torrent; transcript annotations; small test data for experimenting with pipelines (e.g., for new users).

In some embodiments, data is made available within the context of a database included in a system. Any suitable database structure may be used including relational databases, object-oriented databases, and others. In some embodiments, reference data is stored in a relational database such as a “not-only SQL” (NoSQL) database. In various embodiments, a graph database is included within systems of the disclosure. It is also to be understood that the term “database” as used herein is not limited to one single database; rather, multiple databases can be included in a system. For example, a database can include two, three, four, five, six, seven, eight, nine, ten, fifteen, twenty, or more individual databases, including any integer of databases therein, in accordance with embodiments of the disclosure. For example, one database can contain public reference data, a second database can contain test data from a patient, a third database can contain data from healthy subjects, and a fourth database can contain data from sick subjects with a known condition or disorder. It is to be understood that any other configuration of databases with respect to the data contained therein is also contemplated by the methods described herein.

### ILLUSTRATIVE EMBODIMENTS

The present disclosure provides the following illustrative embodiments:
Embodiment 1. A method of sequencing cell-free nucleic acid molecules of a subject, the method comprising:
   (a) treating a urine sample to inhibit cell lysis;
   (b) separating cell-free nucleic acid molecules in the treated urine sample from cells in the treated urine sample, thereby producing a purified urine sample comprising the cell-free nucleic acid molecules;
   (c) concentrating the cell-free nucleic acid molecules in the purified urine sample by passing at least a portion of the purified urine sample through a filter, wherein (i) the concentrating producing a filtrate and a retained urine sample, and (ii) the retained urine sample comprises an increased concentration of cell-free nucleic acid molecules;
   (d) isolating cell-free nucleic acid molecules from the retained urine sample; and
   (e) sequencing the isolated cell-free nucleic acid molecules.
Embodiment 2. The method of Embodiment 1, wherein treating the urine sample to inhibit cell lysis comprises contacting the urine sample with a one or more preservative reagent.
Embodiment 3. The method of Embodiment 1 or 2, wherein the treating includes treatment with a nuclease inhibitor, a formaldehyde quencher, or both.
Embodiment 4. The method of any one of Embodiments 1-3, wherein the treating comprises contacting the urine sample with a composition comprising: (i) imidazolidinyl urea, EDTA, glycine, or a combination thereof; or (ii) sodium azide, EDTA, or a combination thereof.
Embodiment 5. The method of any one of Embodiments 1-4, wherein the separating comprises centrifugation to pellet cells in the treated urine sample.
Embodiment 6. The method of any one of Embodiments 1-5, wherein the filter is substantially impermeable to passage of cell-free nucleic acids, and is substantially permeable to salts in the purified urine sample.
Embodiment 7. The method of any one of Embodiments 1-5, wherein the filter comprises a rated molecular weight cutoff of 10 kD, 5 kD, 3 kD, or lower.
Embodiment 8. The method of any one of Embodiments 1-7, wherein the retained urine sample has a concentration that is increased by at least 2-fold, at least 5-fold, at least 10-fold, or at least 15-fold compared to the purified urine sample.
Embodiment 9. The method of any one of Embodiments 1-8, wherein the retained urine sample has a volume that is at least 50%, at least 75%, or at least 90% lower compared to the volume of the treated urine sample.
Embodiment 10. The method of Embodiment 9, wherein the volume of the treated urine sample is 5 mL, 10 mL, 15 mL, 20 mL, 30 mL, 40 mL, 50 mL, or more.
Embodiment 11. The method of any one of Embodiments 1-10, further comprising freezing the retained urine sample.
Embodiment 12. The method of any one of Embodiments 1-11, wherein the treating is completed within 120, 60, or 30 minutes after collection of the urine sample; and optionally wherein the separating and the concentrating are completed within 7 days after collection.
Embodiment 13. The method of any one of Embodiments 1-12, further comprising amplifying one or more of the isolated cell-free nucleic acid molecules.
Embodiment 14. The method of any one of Embodiments 1-13, further comprising capturing the isolated cell-free nucleic acid molecules, or amplification products thereof, by hybridization to bait oligonucleotides.
Embodiment 15. The method of Embodiment 14, further comprising separating bait-bound cell-free nucleic acid molecules from unbound cell-free nucleic acid molecules.
Embodiment 16. The method of Embodiment 15, wherein each bait oligonucleotide hybridizes to a target genomic region that is differentially methylated in a cancer sample relative to a non-cancer sample.
Embodiment 17. The method of Embodiment 16, wherein the differential methylation comprises at least 80% of CpG sites in the target genomic region being methylated or unmethylated.
Embodiment 18. The method of Embodiment 16 or 17, wherein the cancer is a bladder cancer, a prostate cancer, or a kidney cancer.
Embodiment 19. The method of any one of Embodiments 14-18, wherein each bait oligonucleotide hybridizes to a target genomic region comprising at least five methylation sites.
Embodiment 20. The method of any one of Embodiments 14-19, wherein each bait oligonucleotide hybridizes to a target genomic region comprising a target sequence of a gene selected from Table 1, and wherein the target sequence is at least 25, at least 35, or at least 45 nucleotides in length.
Embodiment 21. The method of Embodiment 20, wherein the target genomic region comprises a target sequence of a gene selected from TWIST1, EOMES, HOXA9, POU4F2, and ZNF154.
Embodiment 22. The method of Embodiment 20, wherein the bait oligonucleotides collectively hybridize to target sequences from at least 10 genes in Table 1.
Embodiment 23. The method of Embodiment 20, wherein the bait oligonucleotides collectively hybridize to target sequences from: (a) genes in Tables 2 or 3; (b) genes in Table 4; or (c) genes in Table 5.
Embodiment 24. The method of any one of Embodiments 1-23, wherein the cell-free nucleic acid molecules comprise cell-free DNA (cfDNA).
Embodiment 25. The method of Embodiment 24, wherein the method further comprises deaminating the cfDNA isolated in step (d) to produce converted cfDNA molecules; optionally wherein the deaminating comprises treatment with a cytosine deaminase or bisulfite.
Embodiment 26. The method of any one of Embodiments 1-25, wherein the method further comprises diagnosing a cancer in the subject.
Embodiment 27. The method of Embodiment 26, wherein the cancer is a bladder cancer, a prostate cancer, or a kidney cancer.
Embodiment 28. The method of Embodiment 26 or 27, wherein the method further comprises treating the cancer in the subject.
Embodiment 29. The method of Embodiment 28, wherein the treating comprises surgical resection, radiation therapy, chemotherapy, and/or immunotherapy.
Embodiment 30. A method of detecting cancer cells in a subject, the method comprising:
   (a) capturing converted cell-free DNA (cfDNA) fragments from a urine sample of the subject, or amplification products thereof, wherein:
      (i) the bait oligonucleotide composition comprises a plurality of different bait oligonucleotides;
      (ii) each bait oligonucleotide of the plurality of different bait oligonucleotides hybridizes to a target sequence of a gene selected from Table 1, wherein the target sequence is at least 25 nucleotides in length;
   (b) separating bait-bound DNA from unbound DNA;
   (c) sequencing the separated DNA to produced sequencing reads; and
   (d) detecting the cancer cells with a trained classifier,
Embodiment 31. The method of Embodiment 30, wherein the trained classifier detects a number of sequencing reads above a threshold for one or more of the target sequences that are identified as hypermethylated and/or hypomethylated in the cfDNA fragments.
Embodiment 32. The method of Embodiment 30 or 31, wherein the bait oligonucleotides are at least 45 nucleotides in length.
Embodiment 33. The method of any one of Embodiments 30-32, wherein the trained classifier discriminates a subject with cancer from a subject without cancer with a defined specificity.
Embodiment 34. The method of Embodiment 33, wherein the classifier is a mixture model classifier.
Embodiment 35. The method of Embodiment 33 or 34, wherein the defined specificity is 0.900 or higher.
Embodiment 36. The method of Embodiment 35, wherein the application of the trained classifier further comprises a sensitivity of 30% or more.
Embodiment 37. The method of any one of Embodiments 30-36, wherein the bait oligonucleotides collectively hybridize to target sequences from at least 10 genes in Table 1.
Embodiment 38. The method of any one of Embodiments 30-36, wherein at least one of the bait oligonucleotides hybridizes to a target sequence of a gene selected from TWIST1, EOMES, HOXA9, POU4F2, and ZNF154.
Embodiment 39. The method of any one of Embodiments 30-36, wherein:
   (a) the cancer cells are bladder cancer cells, and the bait oligonucleotides collectively hybridize to target sequences from genes of Tables 2 or 3;
   (b) the cancer cells are prostate cancer cells, and the bait oligonucleotides collectively hybridize to target sequences from genes of Table 4; or
   (c) the cancer cells are kidney cancer cells, and the bait oligonucleotides collectively hybridize to target sequences from genes of Table 5.
Embodiment 40. The method of any one of Embodiments 30-39, wherein the converted cfDNA molecules comprise cfDNA treated with a cytosine deaminase or bisulfite.
Embodiment 41. The method of any one of Embodiments 30-40, wherein each bait oligonucleotide is conjugated to a solid surface or to a non-nucleotide affinity moiety.
Embodiment 42. The method of any one of Embodiments 30-41, wherein the differential methylation comprises hypermethylation in a cancer sample relative to a non-cancer sample.
Embodiment 43. The method of any one of Embodiments 30-42, wherein each target genomic region comprises at least five methylation sites.
Embodiment 44. The method of any one of Embodiments 30-43, wherein the method further comprises obtaining the converted cfDNA fragments or amplification products thereof, and wherein the obtaining further comprises: (i) treating a urine sample to inhibit cell lysis; (ii) separating cfDNA fragments in the treated urine sample from cells in the treated urine sample, thereby producing a purified urine sample comprising the cfDNA fragments; (iii) concentrating the cfDNA fragments in the purified urine sample by passing at least a portion of the purified urine sample through a filter, wherein the concentrating producing a filtrate and a retained urine sample, and wherein the retained urine sample comprises an increased concentration of cfDNA fragments; and (iv) isolating cfDNA fragments from the retained urine sample.
Embodiment 45. The method of Embodiment 44, further comprising (v) amplifying one or more of the isolated cfDNA fragments.
Embodiment 46. The method of Embodiment 44 or 45, wherein treating the urine sample to inhibit cell lysis comprises contacting the urine sample with a one or more preservative reagent.
Embodiment 47. The method of any one of Embodiments 44-46, wherein the treating includes treatment with a nuclease inhibitor, a formaldehyde quencher, or both.
Embodiment 48. The method of any one of Embodiments 44-47, wherein the treating comprises contacting the urine sample with a composition comprising: (a) imidazolidinyl urea, EDTA, glycine, or a combination thereof; or (b) sodium azide, EDTA, or a combination thereof.
Embodiment 49. The method of any one of Embodiments 44-48, wherein the separating comprises centrifugation to pellet cells in the treated urine sample.
Embodiment 50. The method of any one of Embodiments 44-49, wherein the filter is substantially impermeable to passage of cell-free nucleic acids, and is substantially permeable to salts in the purified urine sample.
Embodiment 51. The method of any one of Embodiments 44-49, wherein the filter comprises a rated molecular weight cutoff of 10 kD, 5 kD, 3 kD, or lower.
Embodiment 52. The method of any one of Embodiments 44-51, wherein the retained urine sample has a concentration that is increased by at least 2-fold, at least 5-fold, at least 10-fold, or at least 15-fold compared to the purified urine sample.
Embodiment 53. The method of any one of Embodiments 44-52, wherein the retained urine sample has a volume that is at least 50%, at least 75%, or at least 90% lower compared to the volume of the treated urine sample.
Embodiment 54. The method of Embodiment 53, wherein the volume of the treated urine sample is 5 mL, 10 mL, 15 mL, 20 mL, 30 mL, 40 mL, 50 mL, or more.
Embodiment 55. The method of any one of Embodiments 44-54, further comprising freezing the retained urine sample.
Embodiment 56. The method of any one of Embodiments 44-55, wherein the treating is completed within 120, 60, or 30 minutes after collection of the urine sample; and optionally wherein the separating and the concentrating are completed within 7 days after collection.
Embodiment 57. The method of any one of Embodiments 30-56, wherein the method further comprises diagnosing a cancer in the subject.
Embodiment 58. The method of Embodiment 57, wherein the cancer is a bladder cancer, a prostate cancer, or a kidney cancer.
Embodiment 59. The method of Embodiment 57 or 58, wherein the method further comprises treating the cancer in the subject.
Embodiment 60. The method of Embodiment 59, wherein the treating comprises surgical resection, radiation therapy, chemotherapy, and/or immunotherapy.
Embodiment 61. A method of treating cancer in a subject, the method comprising selecting a subject having or being at increased risk of developing cancer, and administering a treatment to the subject, wherein:
   (a) the selecting comprises identifying the subject as the source of a urine cell-free DNA (cfDNA) sample comprising one or more differentially methylated target genomic regions above a threshold level for the presence of the cancer;
   (b) the one or more target genomic regions comprise one or more target sequences of one or more genes selected from Table 1;
   (c) each target sequence is at least 25 nucleotides in length;
   (d) the cancer is bladder cancer, prostate cancer, or kidney cancer; and
   (e) the treatment comprises surgical resection, radiation therapy, chemotherapy, immunotherapy, or any combination thereof.
Embodiment 62. The method of Embodiment 61, wherein the threshold level for the presence of the cancer is a level for reference samples from subjects having the cancer.
Embodiment 63. The method of Embodiment 61 or 62, wherein the threshold level for the presence of the cancer is determined by a classifier trained on sequencing reads for converted DNA from subjects having the cancer Embodiment 64. The method of any one of Embodiments 61-63, wherein the one or more target genomic regions comprise target sequences from at least 10 genes in Table 1.
Embodiment 65. The method of any one of Embodiments 61-64, wherein the one or more target genomic regions comprise a target sequence of a gene selected from TWIST1, EOMES, HOXA9, POU4F2, and ZNF154.
Embodiment 66. The method of any one of Embodiments 61-64, wherein:
   (a) the cancer is bladder cancer, and the one or more target genomic regions comprise one or more target sequences of one or more genes selected from Tables 2 or 3;
   (b) the cancer is prostate cancer, and the one or more target genomic regions comprise one or more target sequences of one or more genes selected from Table 4; or
   (c) the cancer is kidney cancer, and the one or more target genomic regions comprise one or more target sequences of one or more genes selected from Table 5.
Embodiment 67. The method of any one of Embodiments 61-66, wherein each target genomic region comprises at least five methylation sites.
Embodiment 68. A composition comprising a plurality of different bait oligonucleotides, wherein:
   (a) the bait oligonucleotides hybridize to converted DNA molecules derived from one or more target genomic regions;
   (b) the one or more target genomic regions comprise one or more target sequences of one or more genes selected from Table 1;
   (c) the one or more target genomic regions are differentially methylated in a cancer; and
   (d) each bait oligonucleotide comprises a sequence at least 25 nucleotides in length that hybridizes to one of the target sequences.
Embodiment 69. The method of Embodiment 68, wherein the one or more target genomic regions comprise target sequences from at least 10 genes selected from Table 1.
Embodiment 70. The method of Embodiment 68 or Embodiment 69, wherein the one or more target genomic regions comprise a target sequence of gene selected from TWIST1, EOMES, HOXA9, POU4F2, and ZNF154.
Embodiment 71. The method of Embodiment 68 or Embodiment 69, wherein the one or more target genomic region comprise target sequences from: (a) genes in Tables 2 or 3; (b) genes in Table 4; or (c) genes in Table 5.
Embodiment 72. The method of any one of Embodiments 68-71, wherein each target genomic region comprises at least five methylation sites.
Embodiment 73. The method of any one of Embodiments 68-72, wherein the differential methylation comprises at least 80% of CpG sites in the target genomic region being methylated or unmethylated.

### EXAMPLES

The following examples are put forth so as to provide those of ordinary skill in the art with a complete disclosure and description of how to make and use the present description, and are not intended to limit the scope of what the inventors regard as their description, nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1—Urine Sample Processing Workflow

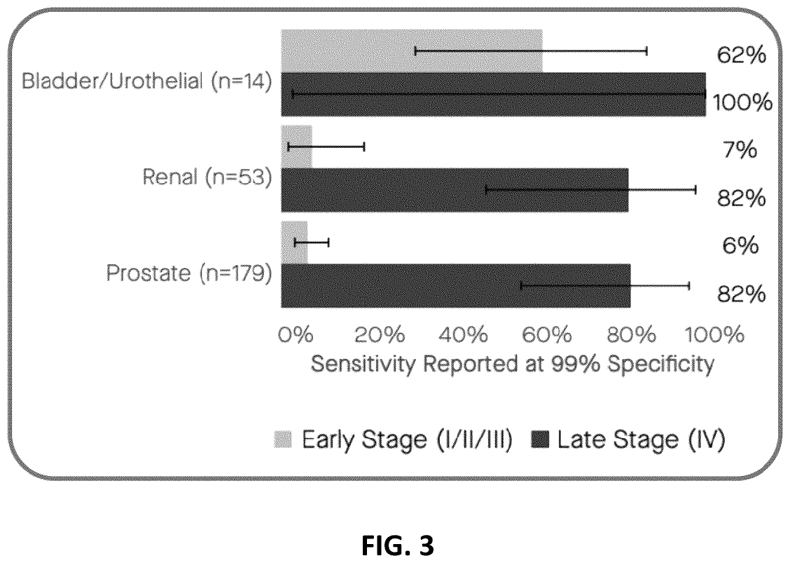

Urological cancers such as prostate, bladder and kidney cancers, have lower detection sensitivity in the Circulating Cell-free Genome Atlas Study (“CCGA”; Clinical Trial.gov identifier NCT02889978), a prospective, multi-center, case-control, observational study with longitudinal follow-up (FIG. 3). This low detection rate may be due to a low tumor fraction of cfDNA in the blood of subjects with urothelial cancers. Analysis of cell-free DNA from urine could increase the sensitivity of detection for urological cancers. Thus, an improved approach to preserve and extract cfDNA from urine was developed.

FIG. 1 shows an exemplary urine sample processing workflow. In this workflow, about 50 mL of urine is collected from a subject. A preservative is then added to the urine sample following collection. Streck Urine Preserve (Streck, Nebraska, USA) or an equivalent preservative containing at least 0.5% weight-by-volume (w/v) of a nuclease inhibitor, at least 0.2-4.0% w/v of a preservative agent, and at least 0.010% w/v of a formaldehyde quencher may be used as the preservative. Other preservatives that may be used include the Urine Collection Medium and UAS (Novosantis, Belgium). Alternatively, urine samples may be collected using a urine collection and preservation device (Norgen Biotek Corp., Canada) or an equivalent cup that contains from 10-30% w/v of a nuclease inhibitor such as EDTA, and 0.1-1.0% w/v of a bacteriostatic preservative agent such as sodium azide.

After addition of the preservative, the urine samples are centrifuged at 4000×g for 20 minutes to sediment and remove any cellular debris. The resulting supernatant is then concentrated approximately 15-fold. The concentration of the preserved urine sample may be achieved using diafiltration column, such as a filter unit having a regenerated cellulose membrane with a 3 kDa cut-off For example, if the resulting supernatant has a volume of 15 mL or lower, the sample may be concentrated by spinning at 4000× g for at least 30 minutes in an Amicon Ultra-15 filter unit (Thermo Fisher Scientific, Massachusetts, US). If working with volumes ranging from 15 mL to 50 mL of supernatant, the sample may be instead concentrated by spinning at 2500× g in a Centricon Plus-70 centrifugal filter unit (Thermo Fisher Scientific, Massachusetts, US), for at least 40 minutes or until the sample has been concentrated to a volume lower than 4.2 mL. The concentration of the urine samples to lower volumes results in samples amenable to automated bead-based extraction methods (e.g., MagMax extraction) and other benchtop accessible techniques.

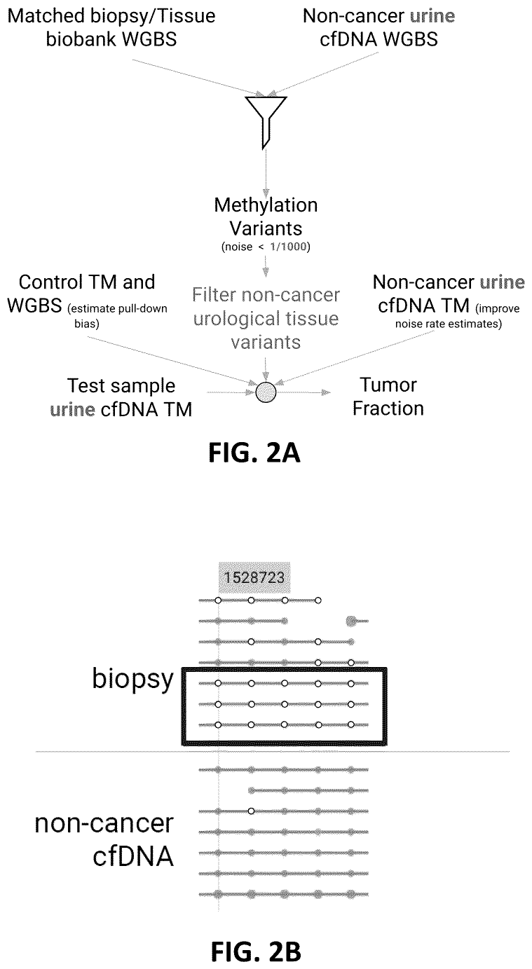

After concentration of the urine sample, the samples may be immediately used or frozen at −80 C for later use, or for batch processing of samples. The concentrated urine sample may then be subjected to cfDNA extraction and library preparation, which can then be sequenced for methylation analysis and urological cancer detection (FIGS. 2 and 4).

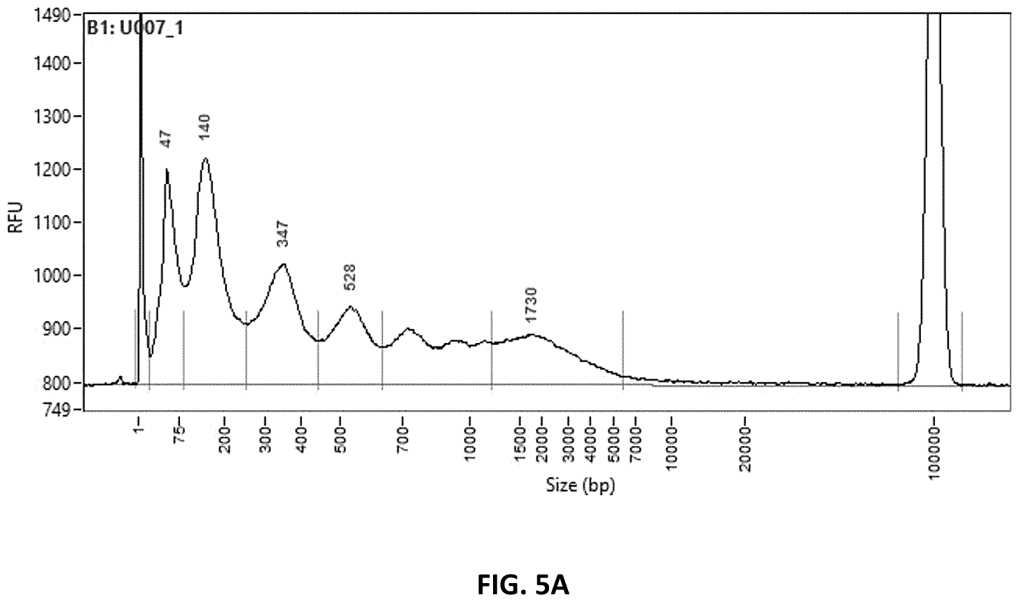

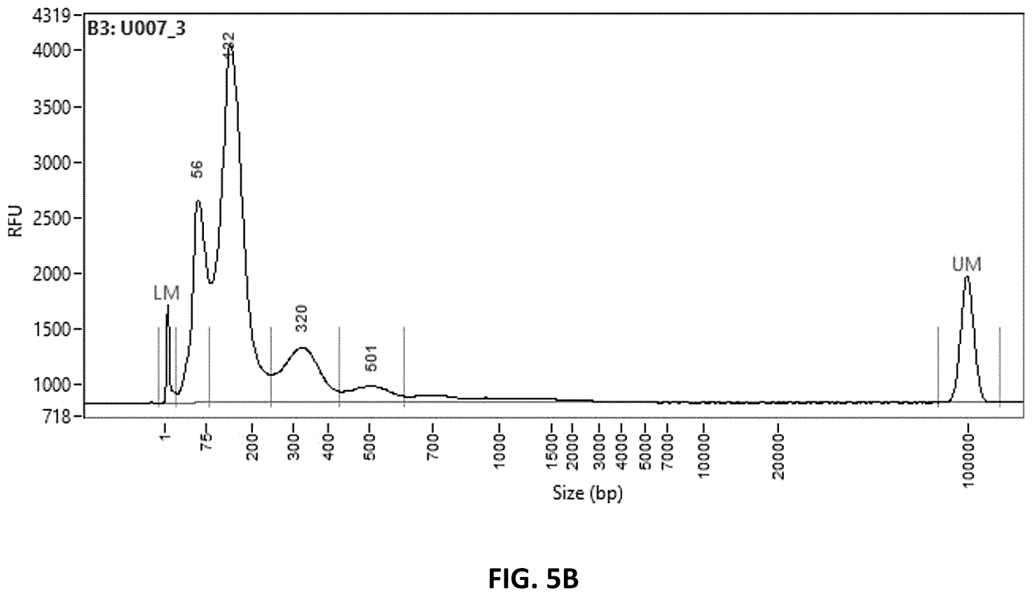

A workflow as illustrated in FIG. 1 was applied to urine samples using the Streck Urine Preserve. Addition of a preservative no more than 30 minutes after urine sample collection preserved nucleosomes and produced cfDNA fragments of up to 7000 bp in size (FIG. 5A). In contrast, delaying the addition of the preservative to an hour or more after collection led to loss of nucleosomal peaks at >700 bp, a significant decrease in yield, and a narrower distribution of fragment lengths skewed toward low molecular weight fragments, indicating lysis of cells in urine, along with degradation and fragmentation of the cfDNA (FIG. 5B). It was also found that by concentrating urine, samples that were frozen for later processing exhibited reduced cryoprecipitate formation.

### Example 2—Analysis of Tumor Fraction in Urine-Derived cfDNA

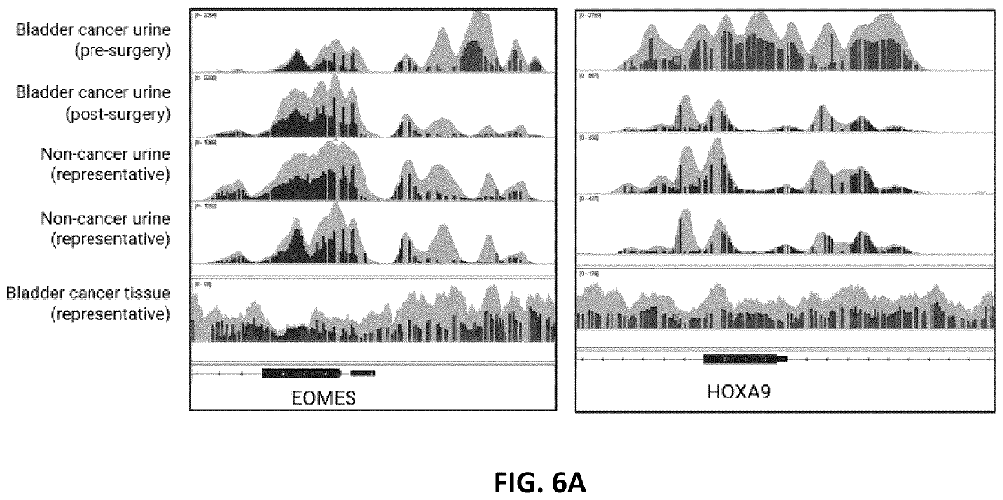

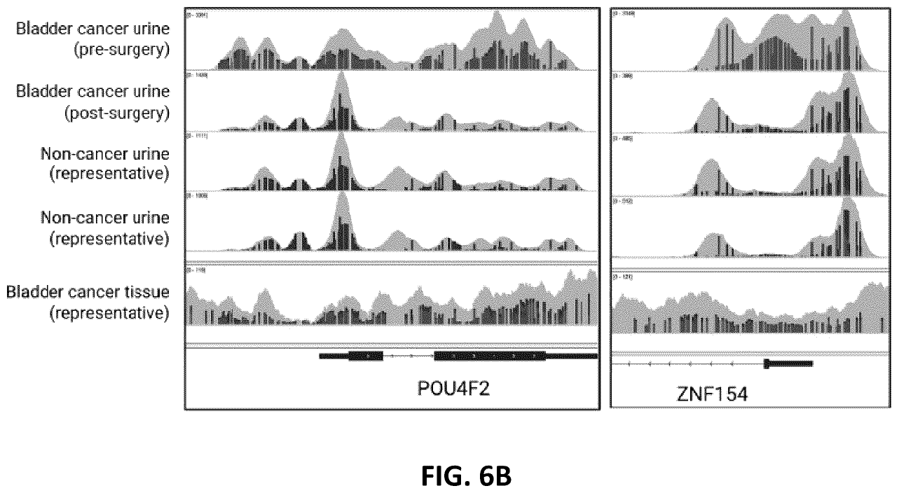

Cancer-specific methylation signatures, such as those corresponding to samples from subjects with stage I, high-grade non-muscle invasive bladder cancer, were detected in urine cfDNA, which was processed from urine samples as described in Example 1 (FIGS. 6A-6B).

A further study was conducted to evaluate detection of cancer-specific methylation markers in urine-derived cfDNA as compared to plasma tumor fraction estimates. Urine and blood were collected from patients with bladder cancer, renal cancer, and prostate cancer as well as from age- and gender-matched non-cancer patients. To generate biopsy-free estimates in urine cfDNA, a plasma-based workflow was modified at the following steps (illustrated in FIG. 2A): (1) an external reference dataset of non-cancer urine cfDNA (N=˜200) was used instead of plasma for the non-cancer WGBS and TM data in the workflow; (2) the noise threshold and pseudocount were adjusted to account for the smaller reference datasets; and (3) WGBS data from healthy urological tissue was used to further filter noisy methylation variants.

Sequencing libraries were then prepared from the resulting cfDNA for methylation analysis of a panel of urological cancer methylation markers. Tumor fraction estimation was also performed using the methylation marker panel, and samples with an estimated tumor fraction above a threshold were identified as detected for cancer based on urine-derived cfDNA. The estimated tumor fraction in urine-derived cfDNA sample was then compared to the estimated tumor fraction from a corresponding plasma-derived cfDNA fraction.

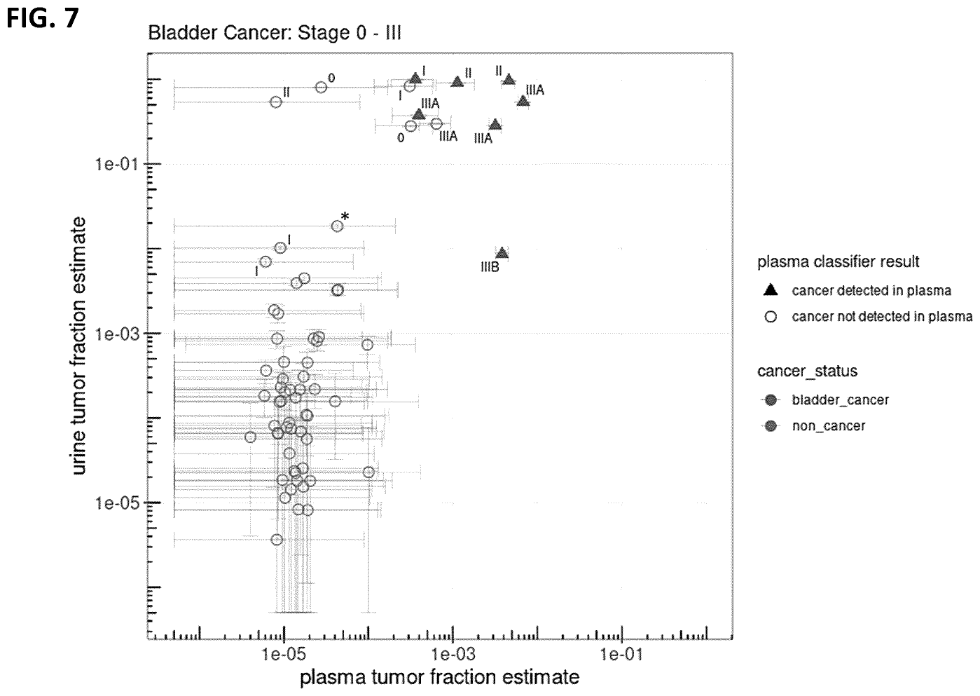

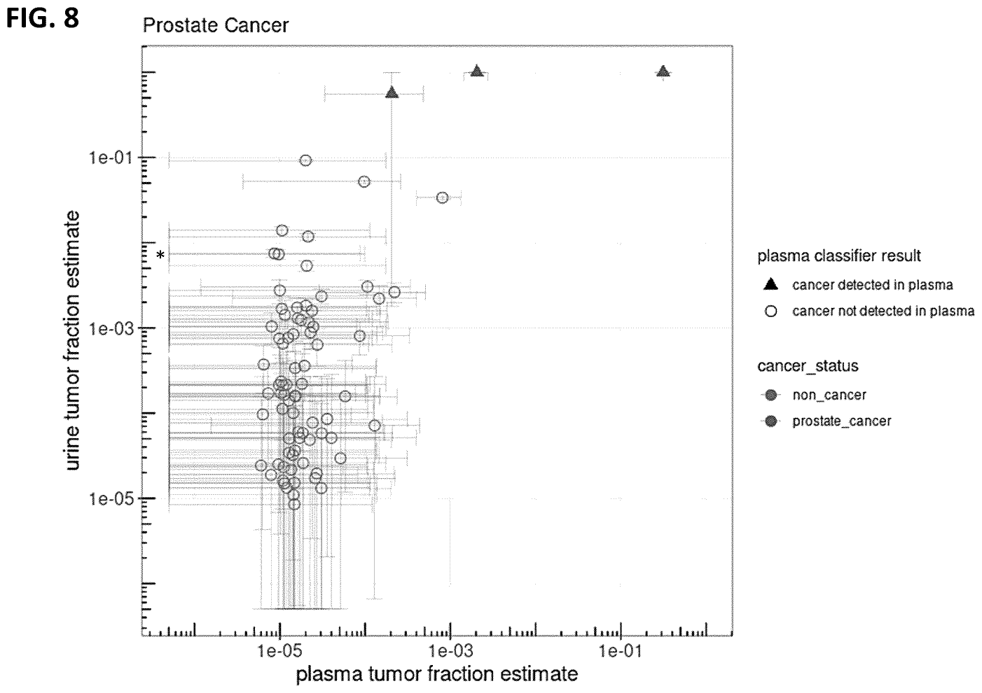

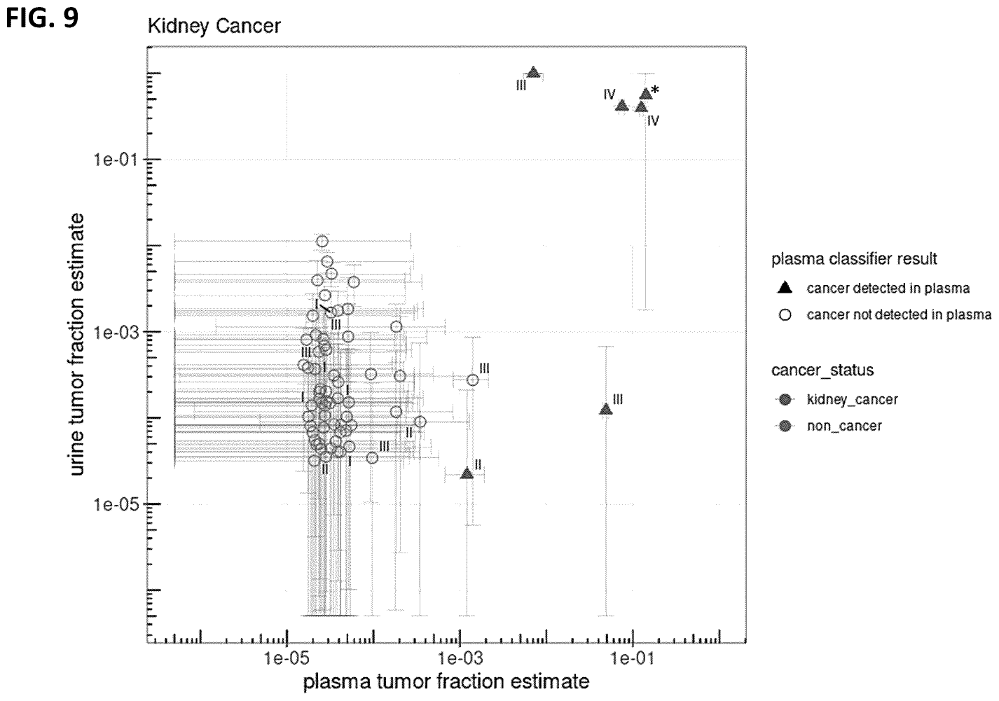

The scatterplots in FIGS. 7-9 show the tumor fraction distribution in each cancer type in matched urine and plasma cfDNA (each point is a single patient). The fill represents whether the multi-cancer classifier (at 99% specificity) detected the plasma cfDNA for that patient. Increased tumor fraction in urine cfDNA relative to plasma was observed in all bladder cancer patients analyzed and in a subset of prostate cancer patients. Plasma tumor fraction estimates were consistent with classifier detection. In kidney cancer patients, while signal in urine was not increased over that of plasma, higher tumor fraction in urine cfDNA relative to non-cancer patients was observed. In a small number of patients, increased signal in plasma relative to urine cfDNA was observed.

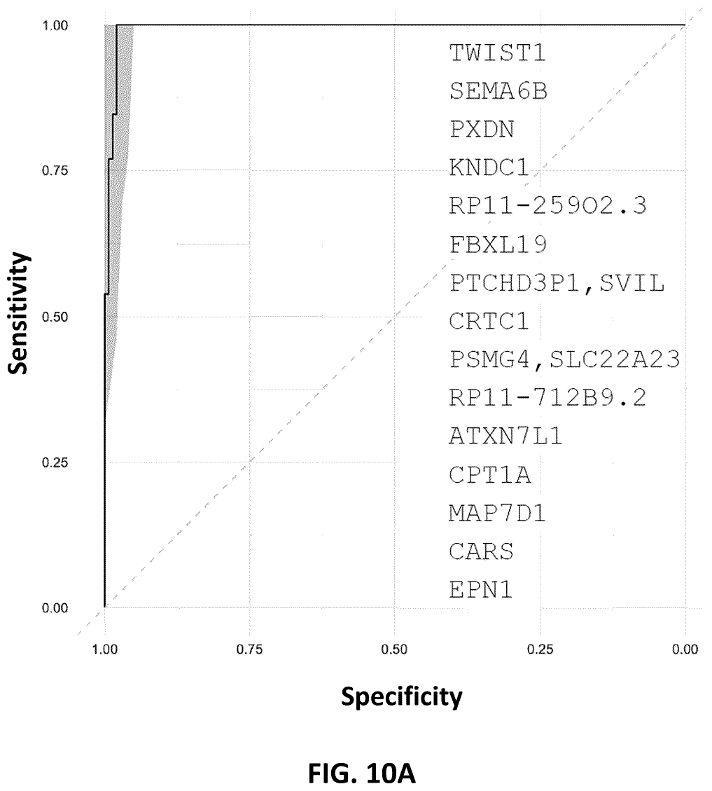

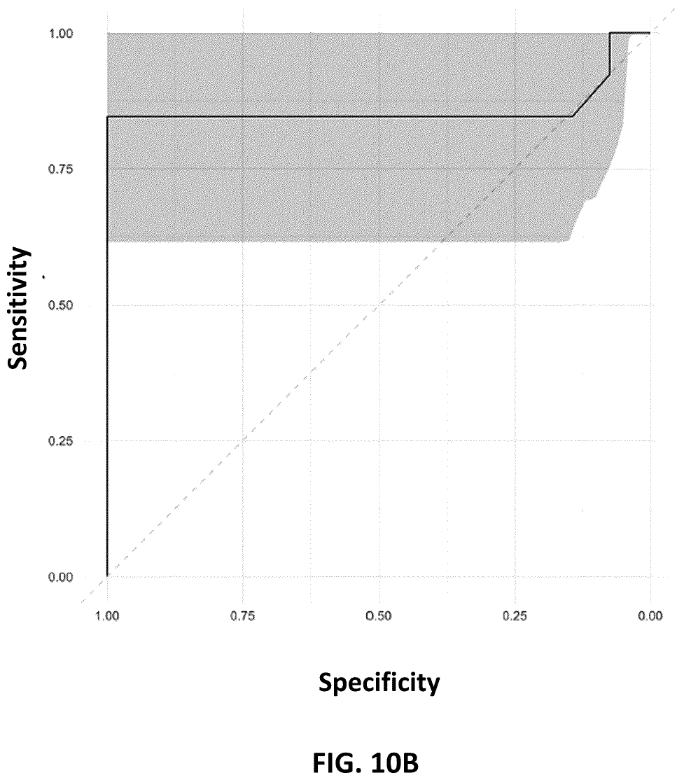

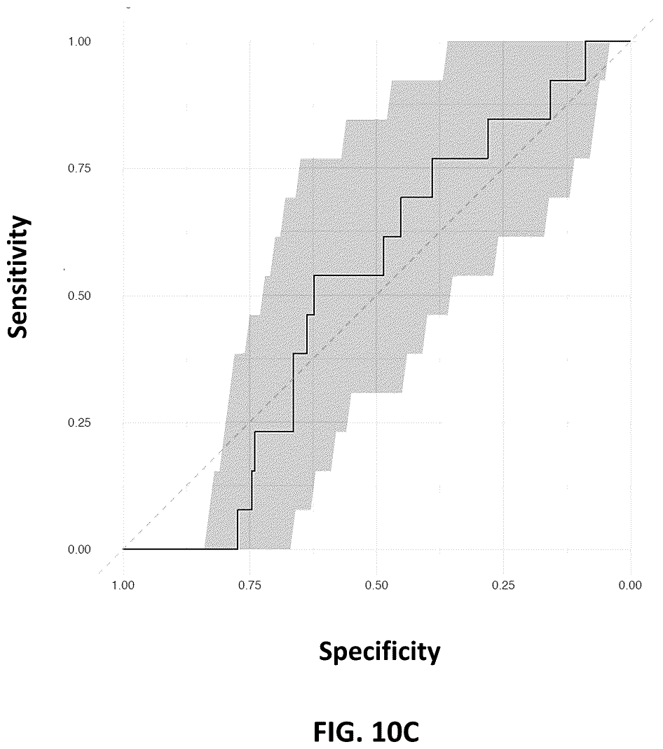

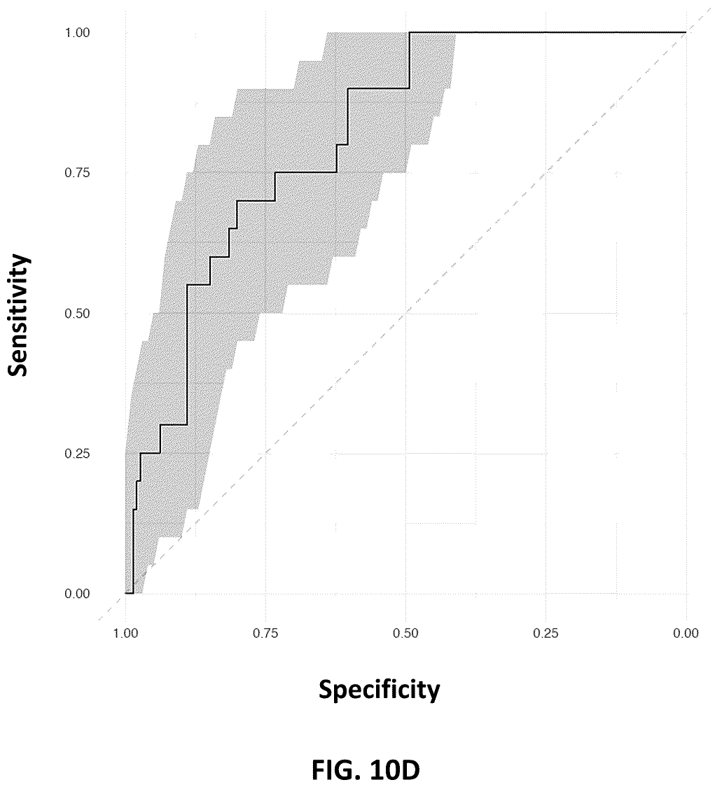

The performance of bladder cancer detection based on a subset of genomic regions in the methylation marker panel was also evaluated. High sensitivity and specificity were observed when detecting bladder cancer status based on a subset of 15 methylation markers (FIG. 10A). Moreover, even when determining bladder cancer status based on only methylation markers within a single gene (TWIST1), high sensitivity and specificity were observed (FIG. 10B).

In addition, the performance of kidney and prostate cancer detection based on a subset of genomic regions in the methylation marker panel was evaluated. The results show that urine-derived cfDNA is diagnostic for both kidney cancer (FIG. 10C) and prostate cancer (FIG. 10D). Genomic regions of the genes in Tables 2 and 3 were found to contain methylation markers of bladder cancer. Genomic regions of the genes in Table 4 were found to contain methylation markers of prostate cancer. Genomic regions of the genes in Table 5 were found to contain methylation markers of kidney cancer. Table 1 presents the union of genes in Tables 2-5. In this example, the genomic region of a gene was deemed to be the sequence from the (actual or putative) transcription start site to the transcription stop site, and an additional 5000 nucleotides from each of these ends. Although the target genomic regions were identified for urine cfDNA sample, such markers may be used for classification of other cfDNA samples, such as cfDNA from other bodily fluids (e.g., blood, serum, or plasma).

In conclusion, determination of urological cancer status based on analysis of methylation markers and tumor fraction was comparable or more accurate when analyzing urine-derived cfDNA than plasma-derived cfDNA. Concentrating urine samples improved the processing and analysis of urine-derived cfDNA for urological cancer detection.

1. A method of sequencing cell-free nucleic acid molecules of a subject, the method comprising:
(a) treating a urine sample to inhibit cell lysis;
(b) separating cell-free nucleic acid molecules in the treated urine sample from cells in the treated urine sample, thereby producing a purified urine sample comprising the cell-free nucleic acid molecules;
(c) concentrating the cell-free nucleic acid molecules in the purified urine sample by passing at least a portion of the purified urine sample through a filter, wherein (i) the concentrating producing a filtrate and a retained urine sample, and (ii) the retained urine sample comprises an increased concentration of cell-free nucleic acid molecules;
(d) isolating cell-free nucleic acid molecules from the retained urine sample; and
(e) sequencing the isolated cell-free nucleic acid molecules.

2. The method of claim 1, wherein treating the urine sample to inhibit cell lysis comprises contacting the urine sample with a one or more preservative reagent.

3. The method of claim 1, wherein the treating includes treatment with a nuclease inhibitor, a formaldehyde quencher, or both.

4. The method of claim 1, wherein the treating comprises contacting the urine sample with a composition comprising: (i) imidazolidinyl urea, EDTA, glycine, or a combination thereof; or (ii) sodium azide, EDTA, or a combination thereof.

5. The method of claim 1, wherein the separating comprises centrifugation to pellet cells in the treated urine sample.

6. The method of claim 1, wherein the filter is substantially impermeable to passage of cell-free nucleic acids, and is substantially permeable to salts in the purified urine sample.

7. The method of claim 1, wherein the filter comprises a rated molecular weight cutoff of 10 kD, 5 kD, 3 kD, or lower.

8. The method of claim 1, wherein the retained urine sample has a concentration that is increased by at least 2-fold, at least 5-fold, at least 10-fold, or at least 15-fold compared to the purified urine sample.

9. The method of claim 1, wherein the retained urine sample has a volume that is at least 50%, at least 75%, or at least 90% lower compared to the volume of the treated urine sample.

10. The method of claim 9, wherein the volume of the treated urine sample is 5 mL, 10 mL, 15 mL, 20 mL, 30 mL, 40 mL, 50 mL, or more.

11. The method of claim 1, further comprising freezing the retained urine sample.

12. The method of claim 1, wherein the treating is completed within 120, 60, or 30 minutes after collection of the urine sample; and optionally wherein the separating and the concentrating are completed within 7 days after collection.

13. The method of claim 1, further comprising amplifying one or more of the isolated cell-free nucleic acid molecules.

14. The method of claim 1, further comprising capturing the isolated cell-free nucleic acid molecules, or amplification products thereof, by hybridization to bait oligonucleotides.

15. The method of claim 14, further comprising separating bait-bound cell-free nucleic acid molecules from unbound cell-free nucleic acid molecules.

16. The method of claim 15, wherein each bait oligonucleotide hybridizes to a target genomic region that is differentially methylated in a cancer sample relative to a non-cancer sample.

17. The method of claim 16, wherein the differential methylation comprises at least 80% of CpG sites in the target genomic region being methylated or unmethylated.

18. The method of claim 16, wherein the cancer is a bladder cancer, a prostate cancer, or a kidney cancer.

19. The method of claim 14, wherein each bait oligonucleotide hybridizes to a target genomic region comprising at least five methylation sites.

20. The method of claim 14, wherein each bait oligonucleotide hybridizes to a target genomic region comprising a target sequence of a gene selected from Table 1, and wherein the target sequence is at least 25, at least 35, or at least 45 nucleotides in length.

21. The method of claim 20, wherein the target genomic region comprises a target sequence of a gene selected from TWIST1, EOMES, HOXA9, POU4F2, and ZNF154.

22. The method of claim 20, wherein the bait oligonucleotides collectively hybridize to target sequences from at least 10 genes in Table 1.

23. The method of claim 20, wherein the bait oligonucleotides collectively hybridize to target sequences from: (a) genes in Tables 2 or 3; (b) genes in Table 4; or (c) genes in Table 5.

24. The method of claim 1, wherein the cell-free nucleic acid molecules comprise cell-free DNA (cfDNA).

25. The method of claim 24, wherein the method further comprises deaminating the cfDNA isolated in step (d) to produce converted cfDNA molecules; optionally wherein the deaminating comprises treatment with a cytosine deaminase or bisulfite.

26. The method of claim 1, wherein the method further comprises diagnosing a cancer in the subject.

27. The method of claim 26, wherein the cancer is a bladder cancer, a prostate cancer, or a kidney cancer.

28. The method of claim 26, wherein the method further comprises treating the cancer in the subject.

29. The method of claim 28, wherein the treating comprises surgical resection, radiation therapy, chemotherapy, and/or immunotherapy.

30. A method of detecting cancer cells in a subject, the method comprising:
(a) capturing converted cell-free DNA (cfDNA) fragments from a urine sample of the subject, or amplification products thereof, wherein:
(i) the bait oligonucleotide composition comprises a plurality of different bait oligonucleotides;
(ii) each bait oligonucleotide of the plurality of different bait oligonucleotides hybridizes to a target sequence of a gene selected from Table 1, wherein the target sequence is at least 25 nucleotides in length;
(b) separating bait-bound DNA from unbound DNA;
(c) sequencing the separated DNA to produced sequencing reads; and
(d) detecting the cancer cells with a trained classifier.

31.-43. (canceled)

44. The method of claim 30, wherein the method further comprises obtaining the converted cfDNA fragments or amplification products thereof, and wherein the obtaining further comprises: (i) treating a urine sample to inhibit cell lysis; (ii) separating cfDNA fragments in the treated urine sample from cells in the treated urine sample, thereby producing a purified urine sample comprising the cfDNA fragments; (iii) concentrating the cfDNA fragments in the purified urine sample by passing at least a portion of the purified urine sample through a filter, wherein the concentrating producing a filtrate and a retained urine sample, and wherein the retained urine sample comprises an increased concentration of cfDNA fragments; and (iv) isolating cfDNA fragments from the retained urine sample.

45.-60. (canceled)

61. A method of treating cancer in a subject, the method comprising selecting a subject having or being at increased risk of developing cancer, and administering a treatment to the subject, wherein:
(a) the selecting comprises identifying the subject as the source of a urine cell-free DNA (cfDNA) sample comprising one or more differentially methylated target genomic regions above a threshold level for the presence of the cancer;
(b) the one or more target genomic regions comprise one or more target sequences of one or more genes selected from Table 1;
(c) each target sequence is at least 25 nucleotides in length;
(d) the cancer is bladder cancer, prostate cancer, or kidney cancer; and
(e) the treatment comprises surgical resection, radiation therapy, chemotherapy, immunotherapy, or any combination thereof.

62.-67. (canceled)

68. A composition comprising a plurality of different bait oligonucleotides, wherein:
(a) the bait oligonucleotides hybridize to converted DNA molecules derived from one or more target genomic regions;
(b) the one or more target genomic regions comprise one or more target sequences of one or more genes selected from Table 1;
(c) the one or more target genomic regions are differentially methylated in a cancer; and
(d) each bait oligonucleotide comprises a sequence at least 25 nucleotides in length that hybridizes to one of the target sequences.

69.-73. (canceled)
